(19) United States
(12) Patent Application Publication
(10) Pub. No.: US 2023/0242941 A1
(43) Pub. Date: Aug. 3, 2023

(54) METHODS AND COMPOSITIONS FOR ADMINISTERING RECOMBINANT VIRAL VECTORS

(71) Applicants: Eric YUEN, Los Altos, CA (US); UNIVERSITY OF IOWA RESEARCH FOUNDATION, Iowa City, IA (US)

(72) Inventors: Yinghua Tang, Iowa City, IA (US); Ziying Yan, Iowa City, IA (US); John F. Engelhardt, Iowa City, IA (US); Eric Yuen, Los Altos, CA (US)

(21) Appl. No.: 18/013,417

(22) PCT Filed: Jun. 30, 2021
(86) PCT No.: PCT/US2021/039860

§ 371 Date: Dec. 28, 2022

Related U.S. Application Data
(60) Provisional application No. 63/046,190, filed on Jun. 30, 2020.

(51) Int. Cl. C12N 15/86; A61K 31/573; A61K 38/13; A61K 31/436; A61K 31/52; A61K 48/00; A61P 11/00

(52) U.S. Cl. CPC C12N 15/86; A61K 31/573; A61K 38/13; A61K 31/436; A61K 31/52; A61K 48/0083; A61P 11/00; C12N 2750/14143

(57) ABSTRACT

Provided herein are methods of transducing a recombinant viral, e.g., retroviral, vector, improving the transgene expression from a recombinant viral vector, and reducing titers of neutralizing antibodies that bind a recombinant viral vector. In general, these methods include administering to a subject a recombinant viral vector and an effective amount of an immunosuppressive regimen.

### CROSS-REFERENCE TO RELATED APPLICATIONS

This application claims the benefit of the filing date of U.S. application No. 63/046,190, filed on Jun. 30, 2020, the disclosure of which is incorporated by reference herein.

### BACKGROUND

Gene therapy using recombinant viral vectors is an emerging treatment modality for various diseases and syndromes, including for treatment of single-gene defects, genetic disease, acquired diseases, and infectious diseases. Recombinant viral vectors, such as parvoviral vectors, retroviral vectors, and adenoviral vectors, have been used in gene therapy as a way of delivering therapeutic transgenes. Parvoviral gene therapy vectors, such as those based on the adeno-associated virus (AAV), have been successfully used for stable gene expression in both animal models and in patients. While recombinant viral gene therapy vectors represent a promising paradigm, limited transduction efficiency of the virus has been an obstacle for the effective use of gene therapy. Another challenge that has hindered the clinical development of viral gene therapy, has been limited expression of the transgene.

A need currently exists for new approaches to improve transduction efficiency of recombinant viral vectors and methods to increase expression of the transgenes such as to improve therapeutic efficacy.

### SUMMARY

The disclosure provides, inter alia, methods, compositions, and kits for transducing a recombinant viral vector, improving transgene expression from a recombinant viral vector, reducing the titer of neutralizing antibodies that bind to a recombinant viral vector, and for treating a disorder (e.g., cystic fibrosis) in a subject in need thereof.

In one aspect, the disclosure features a method of transducing a recombinant viral vector, the vector being administered to a subject in a dosing regimen including at least two doses, the method including administering to the subject the recombinant viral vector and an effective amount of an immunosuppressive regimen including two or more of a calcineurin inhibitor, a glucocorticoid, an antimetabolite, a mammalian target of rapamycin (mTOR) inhibitor, an alkylating agent, a purine biosynthesis inhibitor, an anti-cluster of differentiation 20 (CD20) antibody, a polyclonal anti-lymphocyte antibody, an immunomodulatory drug, or an immunoglobulin protease.

In another aspect, the disclosure features an immunosuppressive regimen including two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in improving transduction of a recombinant viral vector that is administered to a subject in a dosing regimen including at least two doses.

In another aspect, the disclosure features a method of transducing a recombinant viral vector, the vector being administered to a subject in a dosing regimen including at least two doses, the method including administering to the subject the recombinant viral vector and an effective amount of an immunosuppressive regimen including one or more of fingolimod and an immunoglobulin protease.

In another aspect, the disclosure features an immunosuppressive regimen including one or more of fingolimod and an immunoglobulin protease for use in (i) improving transduction of a recombinant viral vector that is administered to a subject in a dosing regimen including at least two doses; (ii) improving transgene expression from a recombinant viral vector that is administered to a subject in a dosing regimen including at least two doses; and/or reducing the titer of neutralizing antibodies that bind to a recombinant viral vector that is administered to a subject in a dosing regimen including at least two doses.

In some aspects, transduction of the recombinant viral vector is improved relative to the transduction level achieved by administration of the recombinant viral vector to the subject in the dosing regimen in the absence of administration of the immunosuppressive regimen.

In some aspects, the immunosuppressive regimen improves transduction by inhibiting an immune response in the subject against the viral vector.

In some aspects, the immune response is an innate immune response, a B-cell mediated immune response, and/or a T-cell mediated immune response.

In some aspects, the immunosuppressive regimen improves viral uptake or improves transduction efficiency of the viral vector.

In another aspect, the disclosure features a method of improving transgene expression from a recombinant viral vector, the vector being administered to a subject in a dosing regimen including at least two doses, the method including administering to the subject an effective amount of an immunosuppressive regimen including two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease.

In another aspect, the disclosure features an immunosuppressive regimen including two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in improving transgene expression from a recombinant viral vector that is administered to a subject in a dosing regimen including at least two doses.

In another aspect, the disclosure features a method of improving transgene expression from a recombinant viral vector, the vector being administered to a subject in a dosing regimen including at least two doses, the method including administering to the subject an effective amount of an immunosuppressive regimen including one or more of fingolimod and an immunoglobulin protease.

In some aspects, transgene expression from the recombinant viral vector is improved relative to the level of transgene expression achieved by administration of the recombinant viral vector to the subject in the dosing regimen in the absence of administration of the immunosuppressive regimen.

In some aspects, transgene expression is improved by about 10-fold, about 20-fold, about 30-fold, about 40-fold, about 50-fold, about 60-fold, about 70-fold, about 80-fold, or about 90-fold relative to the level of transgene expression achieved by administration of the recombinant viral vector to the subject in the dosing regimen in the absence of administration of the immunosuppressive regimen.

In another aspect, the disclosure features a method of reducing the titer of neutralizing antibodies that bind to a recombinant viral vector, the vector being administered to a subject in a dosing regimen including at least two doses, the method including administering to the subject an effective amount of an immunosuppressive regimen including two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease.

In another aspect, the disclosure features an immunosuppressive regimen including two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in reducing the titer of neutralizing antibodies that bind to a recombinant viral vector that is administered to a subject in a dosing regimen including at least two doses.

In another aspect, the disclosure features a method of reducing the titer of neutralizing antibodies that bind to a recombinant viral vector, the vector being administered to a subject in a dosing regimen including at least two doses, the method including administering to the subject an effective amount of an immunosuppressive regimen including one or more of fingolimod and an immunoglobulin protease.

In some aspects, the titer of neutralizing antibodies is reduced relative to the level of neutralizing antibodies resulting from administration of the recombinant viral vector to the subject in the dosing regimen in the absence of administration of the immunosuppressive regimen.

In some aspects, the titer of neutralizing antibodies is reduced about 2-fold, about 3-fold, about 4-fold, or about 5-fold relative to the level of neutralizing antibodies resulting from administration of the recombinant viral vector to the subject in the dosing regimen in the absence of administration of the immunosuppressive regimen.

In some aspects, the immunosuppressive regimen includes two or more of a calcineurin inhibitor, a glucocorticoid, and an antimetabolite.

In some aspects, the immunosuppressive regimen includes a calcineurin inhibitor, a glucocorticoid, and an antimetabolite.

In some aspects, the calcineurin inhibitor is cyclosporine, tacrolimus, or a combination thereof.

In some aspects, the calcineurin inhibitor is cyclosporine.

In some aspects, the glucocorticoid is methylprednisolone, prednisolone, hydrocortisone, dexamethasone, cortisone, budesonide, betamethasone, beclomethasone, triamcinolone, or a combination thereof.

In some aspects, the glucocorticoid is methylprednisolone.

In some aspects, the antimetabolite is a purine analogue, a pyrimidine analogue, a nucleoside analogue, a nucleotide analogue, an antifolate, or a combination thereof.

In some aspects, the purine analogue is azathioprine, mercaptopurine, clofarabine, a thiopurine, fludarabine, pentostatin, cladribine, or a combination thereof.

In some aspects, the purine analogue is azathioprine.

In some aspects, the mTOR inhibitor is rapamycin, everolimus, temsirolimus, ridaforolimus, or a combination thereof.

In some aspects, the alkylating agent is cyclophosphamide.

In some aspects, the purine biosynthesis inhibitor is mycophenolate mofetil (MMF), mycophenolate sodium, or a combination thereof.

In some aspects, the anti-CD20 antibody is rituximab.

In some aspects, the polyclonal anti-lymphocyte antibody is anti-thymocyte globulin (ATG).

In some aspects, the immunomodulatory drug is fingolimod.

In some aspects, the dosing regimen of the recombinant viral vector includes at least a first dose and a second dose of the recombinant viral vector.

In some aspects, the second dose of the recombinant viral vector is administered to the subject at least about 4 weeks after the first dose.

In some aspects, the second dose of the recombinant viral vector is administered to the subject about 4 weeks, about 2 months, about 6 months, or about 12 months after the first dose.

In some aspects, the second dose of the recombinant viral vector is administered to the subject about 4 weeks after the first dose.

In some aspects, the immunosuppressive regimen includes at least a first dose.

In some aspects, the first dose of the immunosuppressive regimen is administered to the subject about 1 day, about 2 days, about 3 days, about 4 days, about 5 days, about 6 days, about 7 days, about 2 weeks, about 4 weeks, or about 8 weeks prior to the first dose of the dosing regimen of the recombinant viral vector.

In some aspects, the first dose of the immunosuppressive regimen is administered to the subject about 2 days prior to the first dose of the dosing regimen of the recombinant viral vector.

In some aspects, the first dose of the immunosuppressive regimen is administered to the subject on the same day as the first dose of the dosing regimen of the recombinant viral vector.

In some aspects, the first dose of the immunosuppressive regimen is administered to the subject about 1 day, about 2 days, about 3 days, about 4 days, about 5 days, about 6 days, about 7 days, about 2 weeks, about 3 weeks, about 4 weeks, or about 8 weeks after the first dose of the dosing regimen of the recombinant viral vector.

In some aspects, the first dose of the immunosuppressive regimen is administered to the subject about 7 days after the first dose of the dosing regimen of the recombinant viral vector.

In some aspects, the immunosuppressive regimen is administered to the subject every day, every two days, every three days, every four days, every five days, every six days, every week, every two weeks, every three weeks, every four weeks, every five weeks, every six weeks, every seven weeks, or every eight weeks.

In some aspects, the immunosuppressive regimen is administered to the subject every day.

In some aspects, the recombinant viral vector is a recombinant parvoviral vector, a recombinant retroviral vector, or a recombinant adenoviral vector.

In some aspects, the recombinant viral vector is a recombinant parvoviral vector.

In some aspects, the parvoviral vector is a recombinant adeno-associated virus (rAAV) or a recombinant bocavirus vector.

In some aspects, the parvoviral vector is an rAAV.

In some aspects, the recombinant parvoviral vector includes a capsid protein and a polynucleotide including an enhancer and/or a promoter operably linked to a transgene.

In some aspects, the capsid protein includes an AV.TL65 capsid protein, an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AVrh.10 capsid protein, a bocavirus capsid protein, a variant thereof, or a combination thereof.

In some aspects, the capsid protein is an AV.TL65 capsid protein or a variant thereof.

In some aspects, the capsid protein is a bocavirus capsid protein.

In some aspects, the capsid protein is a human bocavirus (HBoV) capsid protein.

In some aspects, the human bocavirus capsid protein is an HBoV1 capsid protein, an HBoV2 capsid protein, an HBoV3 capsid protein, or an HBoV4 capsid protein.

In some aspects, the enhancer includes an F5 enhancer or a variant thereof.

In some aspects, the promoter includes a tg83 promoter or a variant thereof.

In some aspects, the transgene is a therapeutic protein.

In some aspects, the therapeutic protein is a CFTR gene (e.g., a human CFTR gene) or a variant thereof.

In some aspects, the therapeutic protein is a CFTRΔR minigene or a variant thereof.

In some aspects, the therapeutic protein is alpha-1 antitrypsin (AAT), surfactant protein (SP)-B, SP-C, or a variant thereof.

In some aspects, the recombinant parvoviral vector is an rAAV including (i) an AV.TL65 capsid protein or a variant thereof; and (ii) a polynucleotide including an F5 enhancer, or a variant thereof, and a tg83 promoter, or a variant thereof, operably linked to a CFTRΔR minigene or a variant thereof.

In some aspects, the AV.TL65 capsid protein includes the amino acid sequence of SEQ ID NO:13 or the variant includes a sequence having at least 80% sequence identity to SEQ ID NO:13.

In some aspects, the F5 enhancer includes the polynucleotide sequence of SEQ ID NO:1 or the variant includes a sequence having at least 80% sequence identity to SEQ ID NO:1.

In some aspects, the F5 enhancer includes the polynucleotide sequence of SEQ ID NO:14 or the variant includes a sequence having at least 80% sequence identity to SEQ ID NO:14.

In some aspects, the tg83 promoter includes the polynucleotide sequence of SEQ ID NO:2 or the variant includes a sequence having at least 80% sequence identity to SEQ ID NO:2.

In some aspects, the CFTRΔR minigene is a human CFTRΔR minigene.

In some aspects, the human CFTRΔR minigene is encoded by a polynucleotide including the sequence of SEQ ID NO:4 or a variant thereof including a sequence having at least 80% sequence identity to SEQ ID NO:4.

In some aspects, the polynucleotide includes, in a 5′-to-3′ direction, the F5 enhancer, the tg83 promoter, and the CFTRΔR minigene.

In some aspects, the recombinant retroviral vector is a recombinant lentiviral vector.

In some aspects, the subject is suffering from a genetic disease, an acquired pulmonary disease, or an infectious disease.

In some aspects, the genetic disease is cystic fibrosis, AAT deficiency, SP-B deficiency, or SP-C deficiency.

In some aspects, the genetic disease is cystic fibrosis.

In some aspects, the acquired pulmonary disease is chronic obstructive pulmonary disorder (COPD).

In some aspects, the infectious disease is a viral infection.

In some aspects, the viral infection is COVID-19.

In some aspects, the method further includes administering one or more additional therapeutic agents to the subject.

In some aspects, the one or more additional therapeutic agents includes an augmenter, an antibiotic, a mucus thinner, a CFTR modulator, a mucolytic, normal saline, hypertonic saline, an immunosuppressive agent, or a combination thereof.

In some aspects, the augmenter includes an anthracycline, a proteasome inhibitor, a tripeptidyl aldehyde, or a combination thereof.

In some aspects, the anthracycline includes doxorubicin, idarubicin, aclarubicin, daunorubicin, epirubicin, calrubicin, mitoxantrone, or a combination thereof.

In some aspects, the proteasome inhibitor includes bortezomib (VELCADE®), carfilzomib, ixazomib, or a combination thereof.

In some aspects, the recombinant viral vector is administered by inhalation, nebulization, aerosolization, intranasally, intratracheally, intrabronchially, orally, intravenously, subcutaneously, or intramuscularly.

In some aspects, the recombinant viral vector is administered by inhalation, nebulization, aerosolization, intranasally, intratracheally, and/or intrabronchially.

In some aspects, the immunosuppressive regimen is administered intraperitoneally, orally, by inhalation, nebulization, aerosolization, intranasally, intratracheally, intrabronchially, intravenously, subcutaneously, or intramuscularly.

In some aspects, the calcineurin inhibitor is administered intraperitoneally.

In some aspects, the glucocorticoid is administered intraperitoneally.

In some aspects, the antimetabolite is administered orally.

In another aspect, the disclosure features a method of administering a recombinant viral vector to a subject, the method including: (a) administering an effective amount of an immunosuppressive regimen including two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease to the subject; and

(b) administering a recombinant viral vector to the subject.

In another aspect, the disclosure features a method of treating cystic fibrosis in a subject in need thereof, the method including: (a) administering an effective amount of an immunosuppressive regimen including two or more of a calcineurin inhibitor, a glucocorticoid, and an antimetabolite to the subject; and (b) administering at least a first dose and a second dose of rAAV including (i) an AV.TL65 capsid protein; and (ii) a polynucleotide including an F5 enhancer and a tg83 promoter operably linked to a CFTRΔR minigene.

In another aspect, the disclosure features an immunosuppressive regimen including two or more of a calcineurin inhibitor, a glucocorticoid, and an antimetabolite for use in treating cystic fibrosis in a subject in need thereof, wherein the immunosuppressive regimen is administered to the subject in combination with at least a first dose and a second dose of rAAV including (i) an AV.TL65 capsid protein; and (ii) a polynucleotide including an F5 enhancer and a tg83 promoter operably linked to a CFTRΔR minigene.

In another aspect, the disclosure features an kit including two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in (i) improving transduction of a recombinant viral vector that is administered to a subject in a dosing regimen including at least two doses; (ii) improving transgene expression from a recombinant viral vector that is administered to a subject in a dosing regimen including at least two doses; and/or reducing the titer of neutralizing antibodies that bind to a recombinant viral vector that is administered to a subject in a dosing regimen including at least two doses.

In another aspect, the disclosure features an kit including one or more of fingolimod and an immunoglobulin protease for use in (i) improving transduction of a recombinant viral vector that is administered to a subject in a dosing regimen including at least two doses; (ii) improving transgene expression from a recombinant viral vector that is administered to a subject in a dosing regimen including at least two doses; and/or reducing the titer of neutralizing antibodies that bind to a recombinant viral vector that is administered to a subject in a dosing regimen including at least two doses.

### BRIEF DESCRIPTION OF THE DRAWINGS

FIG. 1 is a schematic diagram showing an exemplary dosing regimen of AAV2.5T-gaussia luciferase (gLuc), AAV2.5T-ferret CFTRΔR (fCFTRΔR), and the immunosuppressants cyclosporine, methylprednisolone, and azathioprine. AAV2.5T-SP183-fCFTRΔR (ferret CFTRΔR, 1e+13 vg/kg) was combined with proteasome inhibitor, Doxorubicin (Dox, 200 μM). AAV2.5T-SP183-gLuc (gaussia Luciferase, 1e+13 vg/kg) was combined with Dox (200 μM). Immunosuppressants were administered at 2-days before 1^{st} dose and until the day of 2^{nd} dose, total 30 days.

FIGS. 2A and 2B are a series of graphs showing the amount of gLuc as a reporter of transgene expression in the plasma (FIG. 2A) and bronchioalveolar lavage fluid (FIG. 2B) of naïve ferrets or ferrets that were administered AAV2.5T with gLuc and fCFTRΔR transgenes in a single dose, repeat doses, or repeat doses in combination with the immunosuppressants cyclosporine, methylprednisolone, and azathioprine.

FIGS. 3A and 3B are a series of graphs showing the amount of neutralizing antibodies (NAbs) in the plasma (FIG. 3A) and bronchioalveolar lavage fluid (FIG. 3B) of naïve ferrets or ferrets that were administered AAV2.5T-gLuc and AAV2.5T-fCFTRΔR in a single dose, repeat doses, or repeat doses in combination with being administered cyclosporine, methylprednisolone, and azathioprine.

FIGS. 4A-4C are a series of graphs showing the titers of IgG (FIG. 4A). IgM (FIG. 4B), and IgA (FIG. 4C) in the plasma of naïve ferrets or ferrets administered AAV2.5T-fCFTRΔR in a single dose, repeat doses, or repeat doses in combination with being administered cyclosporine, methylprednisolone, and azathioprine.

FIGS. 5A-5C are a series of graphs showing the titers of IgG (FIG. 5A). IgM (FIG. 5B), and IgA (FIG. 5C) in the BALF of naïve ferrets or ferrets administered AAV2.5T-fCFTRΔR in a single dose, repeat doses, or repeat doses in combination with being administered cyclosporine, methylprednisolone, and azathioprine.

### DETAILED DESCRIPTION

Described herein are methods of transducing a recombinant retroviral vector, improving the transgene expression from a recombinant viral vector, and reducing titers of neutralizing antibodies that bind a recombinant viral vector. In general, these methods include administering to a subject the recombinant viral vector and an effective amount of an immunosuppressive regimen. The methods described herein may be used to treat subjects suffering from a genetic disease (e.g., cystic fibrosis), an acquired pulmonary disease (e.g., chronic obstructive pulmonary disorder), or an infectious disease (e.g., COVID-19).

The present disclosure is based, at least in part, on the discovery described herein (see, e.g., Example 1) that administration of an immunosuppressive regimen in combination with a recombinant viral vector resulted in an unexpectedly strong improvement in transduction of the viral vector, along with improved transgene expression, and reduced neutralizing antibody titer. This improvement in viral vector transduction, transgene expression, and reduced neutralizing antibody titer is expected to facilitate improved therapeutic efficacy of recombinant viral vectors carrying therapeutic transgenes, e.g., CFTR or CFTRΔR.

### Definitions

The term ‘AAV’ refers to adeno-associated virus and may be used to refer to the naturally occurring wild-type virus itself or derivatives thereof. The term covers all subtypes, serotypes and pseudotypes, and both naturally occurring and recombinant forms, except where required otherwise. The AAV genome is built of single stranded DNA and comprises inverted terminal repeats (ITRs) at both ends of the DNA strand, and two open reading frames: rep and cap, encoding replication and capsid proteins, respectively. A foreign polynucleotide can replace the native rep and cap genes. AAVs can be made with a variety of different serotype capsids which have varying transduction profiles or, as used herein, “tropism” for different tissue types. As used herein, the term “serotype” refers to an AAV which is identified by and distinguished from other AAVs based on capsid protein reactivity with defined antisera, e.g., AAV1, AAV2, AAV3, AAV4, AAV5, AAV6, AAV7, AAV8, AAV9, and AAVrh10. For example, serotype AAV2 is used to refer to an AAV which contains capsid proteins encoded from the cap gene of AAV2 and a genome containing 5′ and 3′ ITR sequences from the same AAV2 serotype. Pseudotyped AAV as refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped rAAV are produced using standard techniques described in the art.

The term “about” is used herein to mean a value that is ±10% of the recited value.

As used herein, by “administering” is meant a method of giving a dosage of a composition described herein (e.g., a recombinant parvoviral vector or a pharmaceutical composition thereof or an immunosuppressive agent) to a subject. The compositions utilized in the methods described herein can be administered by any suitable route, including, for example, by inhalation, nebulization, aerosolization, intranasally, intratracheally, intrabronchially, orally, parenterally (e.g., intravenously, subcutaneously, or intramuscularly), orally, nasally, rectally, topically, or buccally. In some embodiments, a composition described herein is administered in aerosolized particles intratracheally and/or intrabronchially using an atomizer sprayer (e.g., with a MADgic® laryngo-tracheal mucosal atomization device). The compositions utilized in the methods described herein can also be administered locally or systemically. The method of administration can vary depending on various factors (e.g., the components of the composition being administered, and the severity of the condition being treated).

As used herein, a “combination therapy” or “administered in combination” means that two (or more) different agents or treatments are administered to a subject as part of a defined treatment regimen for a particular disease or condition (e.g., cystic fibrosis). The treatment regimen defines the doses and periodicity of administration of each agent such that the effects of the separate agents on the subject overlap. In some embodiments, the two or more agents are co-formulated. In other embodiments, the two or more agents are not co-formulated. In some embodiments, the delivery of the two or more agents is simultaneous or concurrent. In some embodiments, the two or more agents are administered in a sequential manner as part of a prescribed regimen. In some embodiments, administration of two or more agents or treatments in combination is such that the reduction in a symptom, or other parameter related to the disorder, is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, by inhalation, nebulization, aerosolization, intranasally, intratracheally, intrabronchially, orally, parenterally (e.g., intravenously, subcutaneously, or intramuscularly), orally, nasally, rectally, topically, buccally, or by direct absorption through mucous membrane tissues. The therapeutic agents can be administered by the same route or by different routes. For example, a first therapeutic agent of the combination may be administered by intravenous injection while a second therapeutic agent of the combination may be administered orally.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters (e.g., a tg83 promoter).

An “expression vector” is a vector comprising a region which encodes a polynucleotide or polypeptide of interest and is used for effecting the expression of the polynucleotide or protein in an intended target cell. An expression vector also comprises control elements operatively linked to the encoding region to facilitate expression of the polynucleotide or protein in the target. The combination of control elements and a gene or genes to which they are operably linked for expression is sometimes referred to as an “expression cassette,” a large number of which are known and available in the art or can be readily constructed from components that are available in the art.

A “gene” refers to a polynucleotide containing at least one open reading frame that is capable of encoding a particular protein after being transcribed and translated.

The term “gene delivery” refers to the introduction of an exogenous polynucleotide into a cell for gene transfer, and may encompass targeting, binding, uptake, transport, localization, replicon integration and expression.

The term “gene expression” or “expression” refers to the process of gene transcription, translation, and/or post-translational modification.

The term “gene transfer” refers to the introduction of an exogenous polynucleotide into a cell which may encompass targeting, binding, uptake, transport, localization, and replicon integration, but is distinct from and does not imply subsequent expression of the gene.

The term “gene expression” or “expression” refers to the process of gene transcription, translation, and post-translational modification.

A “helper virus” for AAV refers to a virus that allows AAV (e.g., wild-type AAV) to be replicated and packaged by a mammalian cell. A variety of such helper viruses for AAV are known in the art, including adenoviruses, herpes viruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the American Type Culture Collection (ATCC). Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

As used herein, the term “lentivirus” refers to a genus of the Retroviridae family of viruses that typically gives rise to a slowly developing disease. Viruses included within this group include HIV (human immunodeficiency virus; including HIV type 1 and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which causes immune deficiency and encephalopathy in sub-human primates. Diseases caused by these viruses are typically characterized by a long incubation period and protracted course. Usually, the viruses latently infect monocytes and macrophages from which they spread to other cells. HIV, FIV, and SIV also readily infect T lymphocytes (i.e., T-cells).

The term “lentiviral vector” refers to a vector including one or more nucleic acid sequences that are derived from at least a portion of a lentivirus genome. A lentiviral vector may contain non-coding sequences of one or more proteins from a lentivirus (e.g., HIV-1).

A “lentiviral transfer vector” is a lentiviral vector and includes a heterologous nucleic acid sequence to be transferred into a cell, (e.g., a transgene, including a therapeutic transgene, e.g., a CFTR gene, including a human CFTR gene), as well as, one or more lentiviral genes, or portions thereof. The term encompasses any type of lentiviral transfer vector, including, without limitation, second generation lentiviral transfer vectors (in which transgene expression is driven by the 5′ LTR in a Tat-dependent manner) and third generation lentiviral transfer vectors (in which transgene expression is driven by a chimeric 5′ LTR fused to a heterologous promoter on the transfer plasmid), as well as any modified versions of such lentiviral transfer vectors.

A “lentiviral packaging vector” is a lentiviral vector that includes one or more genes encoding the lentiviral proteins Gag, Pol, or Rev, or portions thereof. For example, in second generation lentiviral packaging systems, the lentiviral packaging vector includes genes encoding the lentiviral proteins Gag, Pol, Rev, and Tat, or portions thereof, on a single plasmid. In third generation lentiviral packaging systems, the genes encoding the Gag and Pol lentiviral proteins, or portions thereof, are included on a single plasmid, while the gene encoding the lentiviral protein Rev, or a portion thereof, is included on a separate plasmid, and the gene encoding the lentiviral protein Tat is eliminated. Transfection of host cells with a transfer vector and one or more packaging vectors can be carried out in order to produce a virus, which can be used to infect target cells thus leading to expression of one or more transgenes.

By “recombinant lentivirus” or “recombinant lentiviral vector” is meant a recombinantly produced lentivirus or lentiviral particle that comprises a polynucleotide sequence not of lentiviral origin (e.g., a polynucleotide comprising a transgene, which may be operably linked to one or more enhancer and/or promoters) such vectors may be delivered into a cell either in vivo, ex vivo, or in vitro. The recombinant lentivirus may use naturally occurring capsid proteins from any lentiviral serotype. In some embodiments, the lentivirus is pseudotyped.

A “detectable marker gene” is a gene that allows cells carrying the gene to be specifically detected (e.g., distinguished from cells which do not carry the marker gene). A large variety of such marker genes are known in the art (e.g., luciferase, lacZ, a fluorescent protein (e.g., green fluorescent protein (GFP), red fluorescent protein (RFP), yellow fluorescent protein (YFP), cyan fluorescent protein (CFP), mCherry, DsRed, and the like).

“Heterologous” means derived from a genotypically distinct entity from that of the rest of the entity to which it is compared. For example, a polynucleotide introduced by genetic engineering techniques into a different cell type is a heterologous polynucleotide (and, when expressed, can encode a heterologous polypeptide).

“Host cells,” “cell lines,” “cell cultures,” “packaging cell line” and other such terms denote eukaryotic cells, e.g., mammalian cells, such as human cells, useful in the present disclosure. These cells can be used as recipients for recombinant vectors, viruses, or other transfer polynucleotides, and include the progeny of the original cell that was transduced. It is understood that the progeny of a single cell may not necessarily be completely identical (in morphology or in genomic complement) to the original parent cell.

As used herein, the term “immunosuppressive regimen” refers to a treatment regimen which includes one or more immunosuppressive agents.

As used herein, the terms “immunosuppressive therapy” and “immunosuppressive agent” refer to a therapy or a therapeutic agent, respectively, that reduces the activation and/or efficacy of the immune system of a subject (e.g., a human). In some embodiments, an immunosuppressive therapy is used to prevent the body from rejecting a transplant (e.g., an organ transplant (e.g., a solid organ transplant) or a bone marrow transplant), to treat graft-versus-host disease after a bone marrow transplant, and/or to treat autoimmune diseases (e.g., systemic lupus erythematosus, rheumatoid arthritis, Crohn's disease, multiple sclerosis, myasthenia gravis, Sarcoidosis, or Behcet's disease). Immunosuppressive agents (also referred to as “immunosuppressants”) include, but are not limited to, a calcineurin inhibitor, a glucocorticoid, an antimetabolite, a mammalian target of rapamycin (mTOR) inhibitor, an alkylating agent, a purine biosynthesis inhibitor, an anti-cluster of differentiation 20 (CD20) antibody, a polyclonal anti-lymphocyte antibody, an immunomodulatory drug, or an immunoglobulin protease, monoclonal antibodies, corticosteroids, biologics, and tyrosine kinase inhibitors. Examples of immunosuppressive agents include, without limitation, cyclosporine, cyclosporine A, cyclosporine G, methylprednisolone, azathioprine, voclosporin, tacrolimus, pimecrolimus, sirolimus, temsirolimus, deforolimus, everolimus, zotarolimus, biolimus, imatinib, dasatinib, nilotinib, erlotinib, sunitinib, gefitinib, bosutinib, neratinib, fingolimod, axitinib, crizotinib, lapatinib, rituximab, toceranib, vatalanib, methotrexate, mycophenolate, cyclophosphamide, and FK506. Additional immunosuppressive agents are described herein or are known in the art.

An “isolated” plasmid, virus, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture.

As used herein, the term “operable linkage” or “operably linked” refers to a physical or functional juxtaposition of the components so described as to permit them to function in their intended manner. More specifically, for example, two DNA sequences operably linked means that the two DNAs are arranged (cis or trans) in such a relationship that at least one of the DNA sequences is able to exert a physiological effect upon the other sequence. For example, an enhancer (e.g., F5) and/or a promoter (e.g., tg83) can be operably linked with a transgene (e.g., a therapeutic transgene, such as a CFTRΔR minigene).

“Packaging” as used herein refers to a series of subcellular events that results in the assembly and encapsidation of a viral vector, particularly an AAV vector. Thus, when a suitable vector is introduced into a packaging cell line under appropriate conditions, it can be assembled into a viral particle. Functions associated with packaging of viral vectors, particularly AAV vectors, are described herein and in the art.

The term “parvovirus” as used herein encompasses the family Parvoviridae, including autonomously-replicating parvoviruses and dependoviruses. The autonomous parvoviruses include members of the genera Parvovirus, Erythrovirus, Bocaparvovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., Fields et al. Virology, 4^{th} ed. Lippincott-Raven Publishers, Philadelphia, 1996. The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. The genus Bocaparvovirus includes bocaviruses HBoV1, HBoV2, HBoV3, and HBoV4.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the disclosure described herein that is a polynucleotide encompasses both the double-stranded form and each of two complementary single-stranded forms known or predicted to make up the double-stranded form.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component. Polypeptides such as “CFTR” and the like, when discussed in the context of gene therapy and compositions therefor, refer to the respective intact polypeptide, or any fragment or genetically engineered derivative thereof that retains the desired biochemical function of the intact protein. Similarly, references to CFTR, CFTRΔR, and other such genes for use in gene therapy (typically referred to as “transgenes” to be delivered to a recipient cell), include polynucleotides encoding the intact polypeptide or any fragment or genetically engineered derivative possessing the desired biochemical function.

By “pharmaceutical composition” is meant any composition that contains a therapeutically or biologically active agent (e.g., a polynucleotide comprising a transgene (e.g., a CFTR gene or a CFTRΔR minigene; see, e.g., Ostedgaard et al. Proc. Natl. Acad. Sci. USA 108(7):2921-6, 2011)), either incorporated into a viral vector (e.g., an rAAV vector) or independent of a viral vector (e.g., incorporated into a liposome, microparticle, or nanoparticle) or an immunosuppressive agent) that is suitable for administration to a subject. Any of these formulations can be prepared by well-known and accepted methods of art. See, for example, Remington: The Science and Practice of Pharmacy (21st ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

By “pharmaceutically acceptable diluent, excipient, carrier, or adjuvant” is meant a diluent, excipient, carrier, or adjuvant which is physiologically acceptable to the subject while retaining the therapeutic properties of the pharmaceutical composition with which it is administered.

“Recombinant,” as applied to a polynucleotide means that the polynucleotide is the product of various combinations of gene synthesis, cloning, restriction and/or ligation steps, and other procedures that result in a construct that is distinct from a polynucleotide found in nature. A recombinant virus is a viral particle comprising a recombinant polynucleotide. The term includes replicates of the original polynucleotide construct and progeny of the original virus construct.

By “recombinant adeno-associated virus (AAV)” or “rAAV vector” is meant a recombinantly-produced AAV or AAV particle that comprises a polynucleotide sequence not of AAV origin (e.g., a polynucleotide comprising a transgene, which may be operably linked to one or more enhancer and/or promoters) to be delivered into a cell, either in vivo, ex vivo, or in vitro. The rAAV may use naturally occurring capsid proteins from any AAV serotype. In some embodiments, non-naturally occurring (e.g., chimeric) capsids may be used in the rAAVs described herein, e.g., AV.TL65.

By “reference” is meant any sample, standard, or level that is used for comparison purposes. A “normal reference sample” or a “wild-type reference sample” can be, for example, a sample from a subject not having the disorder (e.g., cystic fibrosis). A “positive reference” sample, standard, or value is a sample, standard, value, or number derived from a subject that is known to have a disorder (e.g., cystic fibrosis), which may be matched to a sample of a subject by at least one of the following criteria: age, weight, disease stage, and overall health.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. A variety of positive and negative selectable markers are known in the art, some of which are described below.

The terms “subject” and “patient” are used interchangeably herein to refer to any mammal (e.g., a human, a primate, a cat, a dog, a ferret, a cow, a horse, a pig, a goat, a rat, or a mouse). In one embodiment, the subject is a human.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators) and may be comprised of sequence-specific termination sequences or sequence-non-specific terminators or both. A variety of such terminator sequences are known in the art; and illustrative uses of such sequences within the context of the present disclosure are provided below.

A “therapeutic gene,” “prophylactic gene,” “target polynucleotide,” “transgene,” “gene of interest” and the like generally refer to polynucleotide(s) (e.g., a gene or genes) to be transferred using a vector. Such genes may be located within a recombinant viral vector (e.g., a recombinant parvoviral vector. e.g., a rAAV vector (which vector may be flanked by inverted terminal repeat (ITR) regions and thus can be replicated and encapsidated into rAAV particles) or a lentiviral vector). Target polynucleotides can be used in this disclosure to generate recombinant viral vectors for a number of different applications. Such polynucleotides include, but are not limited to: (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; (vi) polynucleotides for cancer therapy, such as E1A tumor suppressor genes or p53 tumor suppressor genes for the treatment of various cancers; and (vii) polynucleotides for gene editing (e.g., CRISPR). To effect expression of the transgene in a recipient host cell, it may be operably linked to a promoter and/or an enhancer, either its own or a heterologous promoter and/or enhancer. A large number of suitable promoters and/or enhancers are known in the art, the choice of which depends on the desired level of expression of the target polynucleotide; whether one desires constitutive expression, inducible expression, cell-specific or tissue-specific expression, etc. The vector may also contain a selectable marker. Exemplary transgenes include, without limitation, cystic fibrosis transmembrane conductance regulator (CFTR) or derivatives thereof (e.g., a CFTRΔR minigene; see, e.g., Ostedgaard et al. Proc. Natl. Acad. Sci. USA 108(7):2921-6, 2011, which is incorporated by reference herein in its entirety), α-antitrypsin, β-globin, γ-globin, tyrosine hydroxylase, glucocerebrosidase, aryl sulfatase A, factor VIII, dystrophin, erythropoietin, alpha 1-antitrypsin, surfactant protein SP-D, SP-A or SP-C, erythropoietin, or a cytokine, e.g., IFN-alpha, IFNγ, TNF, IL-1, IL-17, or IL-6, or a prophylactic protein that is an antigen such as viral, bacterial, tumor or fungal antigen, or a neutralizing antibody or a fragment thereof that targets an epitope of an antigen such as one from a human respiratory virus, e.g., influenza virus or RSV including but not limited to HBoV protein, influenza virus protein, RSV protein, or a coronavirus protein (e.g., a SARS protein or a SARS-CoV-2 protein).

By “therapeutically effective amount” is meant the amount of a composition (e.g., a recombinant viral vector and/or one or more immunosuppressive agents) administered to improve, inhibit, or ameliorate a condition of a subject, or a symptom of a disorder or disease, e.g., cystic fibrosis, in a clinically relevant manner. Any improvement in the subject is considered sufficient to achieve treatment. In one embodiment, an amount sufficient to treat is an amount that reduces, inhibits, or prevents the occurrence or one or more symptoms of a disorder (e.g., cystic fibrosis) or is an amount that reduces the severity of, or the length of time during which a subject suffers from, one or more symptoms of the disorder (e.g., cystic fibrosis) (e.g., by at least about 10%, about 20%, or about 30%, e.g., by at least about 50%, about 60%, or about 70%, or, for example, by at least about 80%, about 90%, about 95%, about 99%, or more, relative to a control subject that is not treated with a composition described herein). An effective amount of the pharmaceutical composition used to practice the methods described herein (e.g., the treatment of cystic fibrosis) varies depending upon the manner of administration and the age, body weight, and general health of the subject being treated. A physician or researcher can decide the appropriate amount and dosage regimen.

“Transduction” or “transducing” as used herein, are terms referring to a process for the introduction of an exogenous polynucleotide, e.g., a transgene in a recombinant viral vector (e.g., an rAAV or a recombinant lentiviral vector), into a host cell leading to expression of the polynucleotide, e.g., the transgene in the cell. With respect to recombinant parvoviral vectors such as an rAAV, the process generally includes 1) endocytosis of the recombinant parvoviral vector (e.g., rAAV) after it has bound to a cell surface receptor, 2) escape from endosomes or other intracellular compartments in the cytosol of a cell, 3) trafficking of the viral particle or viral genome to the nucleus, 4) uncoating of the virus particles, and generation of expressible double stranded parvoviral (e.g., rAAV) genome forms, including circular intermediates. The parvoviral (e.g., rAAV) expressible double stranded form may persist as a nuclear episome or optionally may integrate into the host genome. The alteration of any or a combination of endocytosis of the vector (e.g., rAAV) after it has bound to a cell surface receptor, escape from endosomes or other intracellular compartments to the cytosol of a cell, trafficking of the viral particle or viral genome to the nucleus, or uncoating of the virus particles, and generation of expressive double stranded parvoviral (e.g., rAAV) genome forms, including circular intermediates, may result in altered (e.g., improved) expression levels or persistence of expression, or altered trafficking to the nucleus, or altered types or relative numbers of host cells or a population of cells expressing the introduced polynucleotide, e.g., relative to a reference level. Altered (e.g., improved) expression or persistence of a polynucleotide introduced via a recombinant viral vector (e.g., rAAV) can be determined by methods well known to the art including, but not limited to, protein expression, e.g., by ELISA, flow cytometry and Western blot, measurement of DNA and RNA production by hybridization assays, e.g., Northern blots. Southern blots and gel shift mobility assays, or quantitative or non-quantitative reverse transcription, polymerase chain reaction (PCR), or digital droplet PCR assays.

“Treatment” of an individual or a cell is any type of intervention in an attempt to alter the natural course of the individual or cell at the time the treatment is initiated, e.g., eliciting a prophylactic, curative or other beneficial effect in the individual. For example, treatment of an individual may be undertaken to decrease or limit the pathology caused by any pathological condition, including (but not limited to) an inherited or induced genetic deficiency (e.g., cystic fibrosis), infection by a viral (e.g., SARS-CoV-2), bacterial, or parasitic organism, a neoplastic or aplastic condition, or an immune system dysfunction such as autoimmunity or immunosuppression. Treatment includes (but is not limited to) administration of a composition, such as a pharmaceutical composition, or administration of compatible cells that have been treated with a composition. Treatment may be performed either prophylactically or therapeutically; that is, either prior or subsequent to the initiation of a pathologic event or contact with an etiologic agent. Treatment may reduce one or more symptoms of a pathological condition. For example, symptoms of cystic fibrosis are known in the art and include, e.g., persistent cough, wheezing, breathlessness, exercise intolerance, repeated lung infections, inflamed nasal passages or stuffy nose, foul-smelling or greasy stools, poor weight gain and growth, intestinal blockage, constipation, elevated salt concentrations in sweat, pancreatitis, and pneumonia. Detecting an improvement in, or the absence of, one or more symptoms of a disorder (e.g., cystic fibrosis), indicates successful treatment.

A “variant” refers to a polynucleotide or a polypeptide that is substantially homologous to a native or reference polynucleotide or polypeptide. For example, a variant polynucleotide may be substantially homologous to a native or reference polynucleotide, but which has a polynucleotide sequence different from that of the native or reference polynucleotide because of one or a plurality of deletions, insertions, and/or substitutions. In another example, a variant polypeptide may be substantially homologous to a native or reference polypeptide, but which has an amino acid sequence different from that of the native or reference polypeptide because of one or a plurality of deletions, insertions, and/or substitutions. Variant polypeptide-encoding polynucleotide sequences encompass sequences that comprise one or more additions, deletions, or substitutions of nucleotides when compared to a native or reference polynucleotide sequence, but that encode a variant protein or fragment thereof that retains activity. A wide variety of mutagenesis approaches are known in the art and can be applied by a person of ordinary skill in the art.

A variant polynucleotide or polypeptide sequence can be at least 80%, at least 85%, at least at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a variant sequence can be determined, for example, by comparing the two sequences using freely available computer programs commonly employed for this purpose on the world wide web (e.g., BLASTp or BLASTn with default settings).

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes, and other gene delivery vehicles. The polynucleotide to be delivered, sometimes referred to as a transgene, may comprise a coding sequence of interest in gene therapy (such as a gene encoding a protein of therapeutic or interest), a coding sequence of interest in vaccine development (such as a polynucleotide expressing a protein, polypeptide or peptide suitable for eliciting an immune response in a mammal), and/or a selectable or detectable marker.

### Immunosuppressive Agents and Immunosuppressive Regimens

As described herein, the disclosure provides methods of transducing a recombinant viral vector (e.g., a recombinant parvoviral vector (e.g., an rAAV vector or a bocavirus viral vector) or a recombinant lentiviral vector) that include administering an immunosuppressive regimen to a subject in combination with the recombinant viral vector. Also provided are methods of improving transgene expression from a recombinant viral vector that include administering an immunosuppressive regimen to a subject in combination with the recombinant viral vector. Further provided, are methods of reducing the titer of neutralizing antibodies that bind to a recombinant viral vector that include administering an immunosuppressive regimen to a subject in combination with the recombinant viral vector.

Any suitable immunosuppressive agent or combination of immunosuppressive agents may be used. The immunosuppressive regimen may include one or more (e.g., 1, 2, 3, 4, 5, 6, 7, 8, 9, or 10) immunosuppressive agents selected from a calcineurin inhibitor, a glucocorticoid, an antimetabolite, a mammalian target of rapamycin (mTOR) inhibitor, an alkylating agent, a purine biosynthesis inhibitor, an anti-cluster of differentiation 20 (CD20) antibody, a polyclonal anti-lymphocyte antibody, an immunomodulatory drug, and/or an immunoglobulin protease.

In some embodiments, the immune suppressive regimen includes one or more, two or more, or all three of a calcineurin inhibitor, a glucocorticoid, and an antimetabolite. In some embodiments, the immune suppressive regimen comprises a calcineurin inhibitor, a glucocorticoid, and an antimetabolite.

In some embodiments, one or more of the immunosuppressive agent(s) administered as part of the immunosuppressive regimen is a calcineurin inhibitor. In some embodiments, the calcineurin inhibitor is cyclosporine, tacrolimus, or a combination thereof. In some embodiments, the calcineurin inhibitor agent is cyclosporine.

In some embodiments, one or more of the immunosuppressive agent(s) administered as part of the immunosuppressive regimen is a glucocorticoid (e.g., hydrocortisone, dexamethasone, cortisone, budesonide, beclomethasone, prednisolone, prednisone, triamcinolone, methylprednisolone, and betamethasone). In some embodiments, the glucocorticoid is methylprednisolone, prednisolone, hydrocortisone, dexamethasone, cortisone, budesonide, betamethasone, beclomethasone, triamcinolone, or a combination thereof. In some embodiments, the glucocorticoid is methylprednisolone.

In some embodiments, one or more of the immunosuppressive agent(s) administered as part of the immunosuppressive regimen is an antimetabolite (e.g., a purine analogue (e.g., azathioprine, mercaptopurine, clofarabine, a thiopurine, fludarabine, pentostatin, or cladribine), a pyrimidine analogue, a nucleoside analogue, a nucleotide analogue, an antifolate, fluorouracil, cladribine, methotrexate, mercaptopurine, pemetrexed, gemcitabine, capecitabine, hydroxyurea, fludarabine, pralatrexate, nelarabine, clofarabine, decitabine, cytarabine liposomal, floxuridine, gemcitabine, and thioguanine). In some embodiments, the antimetabolite is a purine analogue, a pyrimidine analogue, a nucleoside analogue, a nucleotide analogue, an antifolate, or a combination thereof. In some embodiments, the purine analogue is azathioprine, mercaptopurine, clofarabine, a thiopurine, fludarabine, pentostatin, cladribine, or a combination thereof. In some embodiments, the purine analogue is azathioprine.

In some embodiments, one or more of the immunosuppressive agent(s) administered as part of the immunosuppressive regimen is an mTOR inhibitor. In some embodiments, the mTOR inhibitor is rapamycin, everolimus, temsirolimus, ridaforolimus, or a combination thereof.

In some embodiments, one or more of the immunosuppressive agent(s) administered as part of the immunosuppressive regimen is an alkylating agent. In some embodiments, the alkylating agent is cyclophosphamide.

In some embodiments, one or more of the immunosuppressive agent(s) administered as part of the immunosuppressive regimen is a purine biosynthesis inhibitor. In some embodiments, the purine biosynthesis inhibitor is mycophenolate mofetil (MMF), mycophenolate sodium, or a combination thereof.

In some embodiments, one or more of the immunosuppressive agent(s) administered as part of the immunosuppressive regimen is an anti-CD20 antibody. In some embodiments, the anti-CD20 antibody is rituximab.

In some embodiments, one or more of the immunosuppressive agent(s) administered as part of the immunosuppressive regimen is a polyclonal anti-lymphocyte antibody. In some embodiments, the polyclonal anti-lymphocyte antibody is an anti-thymocyte globulin (ATG).

In some embodiments, one or more of the immunosuppressive agent(s) administered as part of the immunosuppressive regimen is an immunomodulatory drug. In some embodiments the immunomodulatory drug is fingolimod.

In some embodiments, the immunosuppressive agent may be any agent or combination shown below in Table 1. In other embodiments, the immunosuppressive agent may be from any class of agents shown in Table 1.

TABLE 1
Exemplary Immunosuppressive Agents and Combinations

| Agent/Combination | Class |
|---|---|
| Methylprednisolone | Glucocorticoid |
| Azathioprine | Antimetabolite |
| Cyclosporine (CSP) | Calcineurin inhibitor |
| Methylprednisone + | Glucocorticoid + Antimetabolite + |
| Azathioprine + CSP | Calcineurin inhibitor |
| Rapamycin | mTOR inhibitor |
| Prednisolone | Glucocorticoid |
| Rapamycin + | mTOR inhibitor + Glucocorticoid |
| prednisolone | |
| Cyclophosphamide | Alkylating agent |
| Mycophenolate mofetil | Purine biosynthesis inhibitor |
| (MMF) | |
| MMF + CSP | Purine biosynthesis inhibitor + |
| | calcineurin inhibitor |
| Anti-thymocyte globulin | Anti-T cell antibody |
| (ATG) | |
| ATG + MMF + CSP | Anti-T cell antibody + Purine biosynthesis |
| | inhibitor + Calcineurin inhibitor |
| Rituximab | Anti-CD20 Antibody |
| Tacrolimus | Calcineurin inhibitor |
| Rituximab + rapamycin + | Anti-CD20 antibody + mTOR inhibitor + |
| IV immunoglobulin | IV immunoglobulin |
| MMF + ATG + | Purine biosynthesis inhibitor + Anti-T-cell |
| Methylprednisolone + | antibody + glucocorticoid + Calcineurin |
| tacrolimus + rituximab | inhibitor + anti-CD20 antibody |
| Fingolimod | Immunomodulatory drug (sphingosine- |
| | 1-phosphate receptor modulator) |
| Immunoglobulin (Ig) | Ig protease |
| protease | |

In some embodiments, the immunosuppressive agent is selected from corticosteroids (e.g., an inhaled corticosteroid (e.g., beclomethasone (QVAR®)), budesonide (PULMICORT®), budesonide/formoterol (SYMBICORT®), ciclesonide (ALVESCO®), fluticasone (FLOVENT HFA®)), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), predisone, or methylprednisone), polyclonal anti-lymphocyte antibodies (e.g., anti-lymphocyte globulin (ALG) and anti-thymocyte globulin (ATG) antibodies, which may be, for example, horse- or rabbit-derived), monoclonal anti-lymphocyte antibodies (e.g., anti-CD3 antibodies (e.g., murmomab and alemtuzumab) or anti-CD20 antibodies (e.g., rituximab)), interleukin-2 (IL-2) receptor antagonists (e.g., daclizumab and basiliximab), calcineurin inhibitors (e.g., cyclosporin A and tacrolimus), cell cycle inhibitors (e.g., azathioprine, mycophenolate mofetil (MMF), and mycophenolic acid (MPA)), mammalian target of rapamycin (mTOR) inhibitors (e.g., sirolimus (rapamycin) and everolimus), methotrexate, cyclophosphamide, an anthracycline (e.g., doxorubicin, idarubicin, aclarubicin, daunorubicin, epirubicin, valrubicin, mitoxantrone, or a combination thereof), a taxane (e.g., TAXOL® (paclitaxel)), and a combination thereof (e.g., a combination of a calcineurin inhibitor, a cell cycle inhibitor, and a corticosteroid).

### Recombinant Viral Vectors

Provided herein are recombinant viral vectors that may be administered to a subject in combination with an effective amount of an immunosuppressive regimen. Any suitable recombinant viral vector may be used. In general, the recombinant viral vectors may be administered to the patient in a dosing regimen that includes at least two doses. However, in other embodiments, the recombinant viral vectors may be administered to the patient in one dose. The recombinant viral vector may be a recombinant parvoviral vector (e.g., a recombinant bocavirus vector or a recombinant adeno-associated virus vector), a recombinant retroviral vector (e.g., a lentiviral vector), or a recombinant adenoviral vector. In some embodiments, the recombinant viral vector is a recombinant parvoviral vector. In some embodiments, the parvoviral vector is a recombinant adeno-associated virus (rAAV) or a recombinant bocavirus vector. In some embodiments, the parvoviral vector is an rAAV. The recombinant parvoviral vector (e.g., a recombinant bocavirus vector or an rAAV) may include a capsid protein and a polynucleotide comprising an enhancer and/or a promoter operably linked to a transgene. In some embodiments, the recombinant retroviral vector is a recombinant lentiviral vector (e.g., a recombinant lentiviral transfer vector (e.g., a recombinant lentiviral transfer vector that includes a transgene, e.g., CFTR).

### Recombinant AAV Vectors

Recombinant AAV vectors are potentially powerful tools for human gene therapy. A major advantage of rAAV vectors over other approaches to gene therapy is that they generally do not require ongoing replication of the target cell in order to exist episomally or become stably integrated into the host cell. The rAAV capsid protein may include an AV.TL65 capsid protein, an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AVrh.10 capsid protein, a variant thereof, or a combination thereof. In some embodiments, the capsid protein is an AV.TL65 capsid protein or variant thereof.

In some embodiments, the AV.TL65 capsid protein includes the amino acid sequence of SEQ ID NO:13, or an amino acid sequence having at least 80%, 85%, 90%, 91%, 92%, 93%, 94%, 95%, 96%, 97%, 98%, 99% sequence identity with the amino acid sequence of SEQ ID NO:13.

Any of the rAAV vectors disclosed in International Patent Application No.: PCT/US2020/028264, which is incorporated by reference herein in its entirety, may be used in the methods, compositions, and kits disclosed herein.

Any of the rAAV vectors may include any suitable polynucleotide, including any of the polynucleotides described below.

In some embodiments, the rAAV vector includes an isolated polynucleotide that includes the sequence of SEQ ID NO:7, or a sequence having at least at least 80%, 85%, 90%, 91%, 92%, 93%, 94%, 95%, 96%, 97%, 98%, 99% sequence identity with the polynucleotide sequence of SEQ ID NO:7. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In some embodiments, the polynucleotide further comprises, in the 3′ direction, a 3′ untranslated region (3′-UTR) comprising the sequence of SEQ ID NO:5, or a sequence having at least at least 80%, 85%, 90%, 91%, 92%, 93%, 94%, 95%, 96%, 97%, 98%, 99% sequence identity with the polynucleotide sequence of SEQ ID NO:5.

In some embodiments, the polynucleotide further comprises, in the 3′ direction (e.g., 3′ relative to the 3′-UTR), a synthetic polyadenylation site comprising the sequence of SEQ ID NO:6.

In some embodiments, the polynucleotide further comprises a 5′ adeno-associated virus (AAV) inverted terminal repeat (ITR) at the 5′ terminus of the polynucleotide and/or a 3′ AAV ITR at the 3′ terminus of the polynucleotide. In some embodiments, the polynucleotide comprises the sequence of SEQ ID NO:11, or a sequence having at least at least 80%, 85%, 90%, 91%, 92%, 93%, 94%, 95%, 96%, 97%, 98%, 99% sequence identity with the polynucleotide sequence of SEQ ID NO:11. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In other embodiments, the polynucleotide comprises the sequence of SEQ ID NO:17, or a sequence having at least at least 80%, 85%, 90%, 91%, 92%, 93%, 94%, 95%, 96%, 97%, 98%, 99% sequence identity with the polynucleotide sequence of SEQ ID NO:17. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

Any of the polynucleotides may contain a 5′ AAV ITR. Any suitable 5′ AAV ITR may be used, including a 5′ AAV ITR from any AAV serotype (e.g., AAV2). In some embodiments, the 5′ AAV ITR comprises the sequence of SEQ ID NO:9, or a sequence having at least at least 80%, 85%, 90%, 91%, 92%, 93%, 94%, 95%, 96%, 97%, 98%, 99% sequence identity with the polynucleotide sequence of SEQ ID NO:9. In another example, in some embodiments, the polynucleotide includes a 5′ AAV ITR comprising the sequence of SEQ ID NO:15, or a sequence having at least at least 80%, 85%, 90%, 91%, 92%, 93%, 94%, 95%, 96%, 97%, 98%, 99% sequence identity with the polynucleotide sequence of SEQ ID NO:15. Any of the polynucleotides may contain a 3′ AAV ITR. Any suitable 3′ AAV ITR may be used, including a 3′ AAV ITR from any AAV serotype (e.g., AAV2). In some embodiments, the 3′ AAV ITR comprises the sequence of SEQ ID NO:10, or a sequence having at least at least 80%, 85%, 90%, 91%, 92%, 93%, 94%, 95%, 96%, 97%, 98%, 99% sequence identity with the polynucleotide sequence of SEQ ID NO:10. In another example, in some embodiments, the polynucleotide includes a 3′ AAV ITR comprising the sequence of SEQ ID NO:16, or a sequence having at least at least 80%, 85%, 90%, 91%, 92%, 93%, 94%, 95%, 96%, 97%, 98%, 99% sequence identity with the polynucleotide sequence of SEQ ID NO:16. The ITR sequences may be palindromic, e.g., as in SEQ ID NO:15 and SEQ ID NO:16, where the ITR sequence on the 5′ end is located on the reverse strand, and the ITR sequence on the 3′ end is located on the forward strand.

Any of the polynucleotides may contain an F5 enhancer. See, e.g., U.S. patent application Ser. No. 16/082,767, which is incorporated herein by reference in its entirety. In some embodiments, the F5 enhancer comprises the sequence of SEQ ID NO:1 or SEQ ID NO:14, or a sequence having at least at least 80%, 85%, 90%, 91%, 92%, 93%, 94%, 95%, 96%, 97%, 98%, 99% sequence identity with the polynucleotide sequence of SEQ ID NO:1 or SEQ ID NO:14. In some embodiments, the F5 includes the polynucleotide sequence of SEQ ID NO:1. In other embodiments, the F5 enhancer includes the polynucleotide sequence of SEQ ID NO:14.

Any of the polynucleotides may contain a tg83 promoter. See, e.g., U.S. patent application Ser. No. 16/082,767. In some embodiments, the tg83 promoter comprises the sequence of SEQ ID NO:2, or a sequence having at least at least 80%, 85%, 90%, 91%, 92%, 93%, 94%, 95%, 96%, 97%, 98%, 99% sequence identity with the polynucleotide sequence of SEQ ID NO:2.

Any of the polynucleotides may contain a 5′-UTR. Any suitable 5′-UTR may be used. In some embodiments, the 5′-UTR comprises the sequence of SEQ ID NO:3, or a sequence having at least at least 80%, 85%, 90%, 91%, 92%, 93%, 94%, 95%, 96%, 97%, 98%, 99% sequence identity with the polynucleotide sequence of SEQ ID NO:3.

Any of the polynucleotides may contain a sequence encoding a CFTRΔR minigene. Any suitable CFTRΔR minigene may be used, including human CFTRΔR (hCFTRΔR) or ferret CFTRΔR. In some embodiments, the sequence encoding an hCFTRΔR minigene comprises the sequence of SEQ ID NO:4, or a sequence having at least at least 80%, 85%, 90%, 91%, 92%, 93%, 94%, 95%, 96%, 97%, 98%, 99% sequence identity with the polynucleotide sequence of SEQ ID NO:4.

Any of the polynucleotides may contain a 3′-UTR. Any suitable 3′-UTR may be used. In some embodiments, the 3′-UTR comprises the sequence of SEQ ID NO:3, or a sequence having at least at least 80%, 85%, 90%, 91%, 92%, 93%, 94%, 95%, 96%, 97%, 98%, 99% sequence identity with the polynucleotide sequence of SEQ ID NO:5.

Any of the polynucleotides may contain a polyadenylation site. Any suitable polyadenylation site may be used. In some embodiments, the polyadenylation site comprises the sequence of SEQ ID NO:6, or a sequence having at least at least 80%, 85%, 90%, 91%, 92%, 93%, 94%, 95%, 96%, 97%, 98%, 99% sequence identity with the polynucleotide sequence of SEQ ID NO:6.

In one aspect, the disclosure provides an isolated polynucleotide that includes the sequence of SEQ ID NO:8, or a sequence having at least at least 80%, 85%, 90%, 91%, 92%, 93%, 94%, 95%, 96%, 97%, 98%, 99% sequence identity with the polynucleotide sequence of SEQ ID NO:8. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In one aspect, the disclosure provides an isolated polynucleotide that includes the sequence of SEQ ID NO:11, or a sequence having at least at least 80%, 85%, 90%, 91%, 92%, 93%, 94%, 95%, 96%, 97%, 98%, 99% sequence identity with the polynucleotide sequence of SEQ ID NO:11. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In one aspect, the disclosure provides an isolated polynucleotide that includes the sequence of SEQ ID NO:12, or a sequence having at least at least 80%, 85%, 90%, 91%, 92%, 93%, 94%, 95%, 96%, 97%, 98%, 99% sequence identity with the polynucleotide sequence of SEQ ID NO:12. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In another aspect, the disclosure provides an isolated polynucleotide that includes the sequence of SEQ ID NO:18, or a sequence having at least at least 80%, 85%, 90%, 91%, 92%, 93%, 94%, 95%, 96%, 97%, 98%, 99% sequence identity with the polynucleotide sequence of SEQ ID NO:18. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

The polynucleotide may also contain one or more detectable markers. A variety of such markers are known, including, by way of illustration, the bacterial beta-galactosidase (lacZ) gene; the human placental alkaline phosphatase (AP) gene and genes encoding various cellular surface markers which have been used as reporter molecules both in vitro and in vivo. The polynucleotide may also contain one or more selectable markers.

For example, in some embodiments, the disclosure provides an rAAV that includes (i) an AV.TL65 capsid protein or variant thereof; and (ii) a polynucleotide including an F5 enhancer, or variant thereof, and a tg83 promoter, or variant thereof, operably linked to a CFTRΔR minigene, or a variant thereof.

In some embodiments, the polynucleotide includes, in a 5′-to-3′ direction, the F5 enhancer, the tg83 promoter, and the CFTRΔR minigene. In some particular embodiments, the polynucleotide comprises, in a 5′-to-3′ direction, a 5′ AAV ITR (e.g., an AAV2 5′ ITR), the F5 enhancer, the tg83 promoter, a 5′ untranslated region (UTR), the CFTRΔR minigene, a 3′-UTR, a polyadenylation site, and a 3′ AAV ITR (e.g., an AAV2 3′ ITR).

In some embodiments, the rAAV comprises an AV.TL65 capsid protein. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

A heterologous polynucleotide may be integrated by recombinant techniques into or in place of the AAV genomic coding region (i.e., in place of the AAV rep and cap genes) but is generally flanked on either side by AAV inverted terminal repeat (ITR) regions. This means that an ITR appears both upstream and downstream from the coding sequence, either in direct juxtaposition, in one embodiment (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present disclosure.

The native promoters for rep are self-regulating and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters are some because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1CP4; as well as vaccinia or poxvirus inducible promoters.

Given the relative encapsidation size limits of various AAV genomes, insertion of a large heterologous polynucleotide into the genome necessitates removal of a portion of the AAV sequence. Removal of one or more AAV genes is in any case desirable, to reduce the likelihood of generating replication-competent AAV (“RCA”). Accordingly, encoding or promoter sequences for rep, cap, or both, may be removed, since the functions provided by these genes can be provided in trans.

The resultant vector is referred to as being “defective” in these functions. In order to replicate and package the vector, the missing functions are complemented with a packaging gene, or a plurality thereof, which together encode the functions for the various missing rep and/or cap gene products. The packaging genes or gene cassettes may not be flanked by AAV ITRs and may not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology may be employed since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this disclosure in a number of different forms. Viral particles, plasmids, and stably transformed host cells can all be used to introduce such constructs into the packaging cell, either transiently or stably.

In certain embodiments of this disclosure, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (e.g., inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this disclosure. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., (WO 95/13392); Burstein et al. (WO 98/23018); and Johnson et al. (U.S. Pat. No. 5,656,785)). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this disclosure.

Approaches for producing rAAVs, e.g., rAAVs that contain AV.TL65 capsid proteins are known in the art. See, e.g., Excoffon et al. Proc. Natl. Acad. Sci. USA 106(10):3865-3870, 2009 and U.S. Pat. No. 10,046,016, each of which is incorporated herein by reference in its entirety.

### Recombinant Bocavirus Vectors

Recombinant bocavirus vectors are also useful for human gene therapy. Described herein are recombinant bocavirus vectors that may be administered to a subject in combination with an effective amount of an immunosuppressive regimen. The recombinant bocavirus vector may include a capsid protein and a polynucleotide comprising an enhancer and/or a promoter operably linked to a transgene. The capsid protein may include a bocavirus capsid protein or a variant thereof. In some embodiments, the bocavirus capsid protein is a human bocavirus (HBoV) capsid protein. The HBoV capsid protein may be an HBoV1 capsid protein, an HBoV2 capsid protein, an HBoV3 capsid protein, or an HBoV4 capsid protein. The recombinant bocavirus vector may be, for example, any vector described in WO 2017/205739, which is incorporated by reference herein in its entirety.

In some embodiments, the recombinant parvovirus vector may be a chimeric AAV/bocavirus vector. For example, the chimeric AAV/bocavirus vector may be any chimeric AAV/bocavirus vector described in US 2018/0282702, which is incorporated by reference herein in its entirety. Such recombinant AAV/bocavirus vectors may be produced using any suitable approach. For example, the chimeric AAV/bocavirus vector may be produced using any approach described in WO 2017/139381, which is incorporated by reference herein in its entirety.

### Enhancers

In some embodiments, any one of the parvoviral vectors described herein includes a capsid protein and a polynucleotide including an enhancer and/or promoter operably linked to a transgene. Any suitable enhancer may be used. In some embodiments, the enhancer is an F5 enhancer or variant thereof. In some embodiments, the F5 enhancer includes the polynucleotide sequence of SEQ ID NO:1 or SEQ ID NO:14, or a sequence having at least 80%, 85%, 90%, 91%, 92%, 93%, 94%, 95%, 96%, 97%, 98%, 99% sequence identity with the polynucleotide sequence of SEQ ID NO:1 or SEQ ID NO:14. In some embodiments, the F5 includes the polynucleotide sequence of SEQ ID NO:1. In other embodiments, the F5 enhancer includes the polynucleotide sequence of SEQ ID NO:14.

### Promoters

In some embodiments, the parvoviral vector described herein includes a polynucleotide including an enhancer and/or promoter operably linked to a transgene. Any suitable promoter may be used. In some embodiments, the promoter is a tg83 promoter. In some embodiments, the tg83 promoter includes the polynucleotide sequence of SEQ ID NO:2, or a sequence having at least 80%, 85%, 90%, 91%, 92%, 93%, 94%, 95%, 96%, 97%, 98%, 99% sequence identity with the polynucleotide sequence of SEQ ID NO:2.

### Transgene

Any suitable transgene(s) may be included in the vector, including any transgene described herein or known in the art.

In some embodiments, the parvoviral vector described herein includes a polynucleotide including an enhancer and/or promoter operably linked to a transgene. In some embodiments, the transgene is a therapeutic protein. Any suitable therapeutic protein may be used. In some embodiments, the therapeutic protein is a CFTRΔR minigene or a variant thereof. Any suitable CFTRΔR minigene or a derivative thereof may be used. In some embodiments, the CFTRΔR minigene is a human CFTRΔR minigene. In other embodiments, the CFTRΔR minigene is a ferret CFTRΔR minigene. In some embodiments, the human CFTRΔR minigene is encoded by a polynucleotide including the sequence of SEQ ID NO:4, or a sequence having at least 80%, 85%, 90%, 91%, 92%, 93%, 94%, 95%, 96%, 97%, 98%, 99% sequence identity with the polynucleotide sequence of SEQ ID NO:4.

In other embodiments, the therapeutic protein is alpha-1-antitrypsin (AAT), surfactant protein (SP)-B, SP-C, or a variant thereof

### Recombinant Lentiviral Vectors

In some embodiments, a recombinant lentiviral vector may be administered to the subject in combination with an immunosuppressive regimen. Any suitable lentiviral vector may be used. In some embodiments, the recombinant lentiviral vector includes a polynucleotide that comprises a therapeutic transgene (e.g., any transgene disclosed herein). In some embodiments, the recombinant lentiviral vector includes a polynucleotide that comprises a CFTR gene or a variant thereof (e.g., a human CFTR gene).

### Recombinant Adenoviral Vectors

In some embodiments, a recombinant adenoviral vector may be administered to the subject in combination with an immunosuppressive regimen. Any suitable recombinant adenoviral vector may be used. In some embodiments, the recombinant adenoviral vector includes a polynucleotide that comprises a therapeutic transgene (e.g., any transgene disclosed herein). In some embodiments, the recombinant adenoviral vector includes a polynucleotide that comprises a CFTR gene or a variant thereof (e.g., a human CFTR gene).

### Pharmaceutical Compositions

The disclosure provides pharmaceutical compositions, including pharmaceutical compositions that include any of the recombinant viral vectors or immunosuppressive agents described herein. The pharmaceutical carrier may include one or more pharmaceutically acceptable carriers, excipients, diluents, buffers, and the like.

For example, in one aspect, the disclosure provides a pharmaceutical composition that includes an rAAV, the rAAV including (i) an AV.TL65 capsid protein; and (ii) a polynucleotide including an F5 enhancer and a tg83 promoter operably linked to a CFTRΔR minigene that is administered with a pharmaceutical composition that includes an immunosuppressive regimen.

The pharmaceutical compositions described herein may include a recombinant viral vector, or a recombinant viral vector with one or more additional therapeutic agents. In other examples, the pharmaceutical composition may include one or more immunosuppressive agent(s), including any immunosuppressive agents disclosed herein.

The recombinant viral vector and/or immunosuppressive regimen may be administered to a subject with one or more additional therapeutic agents. Exemplary additional therapeutic agents include, without limitation, an augmenter (e.g., any augmenter described herein, e.g., doxorubicin or idarubicin), an antibiotic (e.g., azithromycin (ZITHROMAX®), amoxicillin and clavulanic acid (AUGMENTIN®), cloxacillin and dicloxacillin, ticarcillin and clavulanic acid (TIMENTIN®), cephalexin, cefdinir, cefprozil, cefaclor; sulfamethoxazole and trimethoprim (BACTRIM®), erythromycin/sulflsoxazole, erythromycin, clarithromycin, tetracycline, doxycycline, minocycline, tigecycline, vancomycin, imipenem, meripenem, Colistimethate/COLISTIN®, linezolid, ciprofloxacin, levofloxacin, or a combination thereof), a mucus thinner (e.g., hypertonic saline or domase alfa (PULMOZYME®)), a CFTR modulator (e.g., ivacaftor (KALYDECO®), lumacaftor, lumacaftor/ivacaftor (ORKAMBI®), tezacaftor/ivacaftor (SYMDEKO®), or TRIKAFTA® (elexacaftor/ivacaftor/tezacaftor)), a mucolytic (e.g., acetylcysteine, ambroxol, bromhexine, carbocisteine, erdosteine, mecysteine, and domase alfa), an immunosuppressive agent, normal saline, hypertonic saline, or a combination thereof. In some embodiments, the one or more additional therapeutic agents includes an augmenter, an antibiotic, a mucus thinner, a CFTR modulator, a mucolytic, normal saline, hypertonic saline, an immunosuppressive agent, or a combination thereof.

Any suitable augmenter may be used. In some embodiments, the augmenter comprises an anthracycline, a proteasome inhibitor, a tripeptidyl aldehyde, or a combination thereof. In some embodiments, anthracycline comprises doxorubicin, idarubicin, aclarubicin, daunorubicin, epirubicin, calrubicin, mitoxantone, or a combination thereof. In some embodiments, the proteasome inhibitor comprises bortezomib (VELCADE®), carfilzomib, ixazomib, or a combination thereof.

Typically, the viral vectors are in a pharmaceutically suitable pyrogen-free buffer such as Ringers balanced salt solution (pH 7.4). Although not required, pharmaceutical compositions may optionally be supplied in unit dosage form suitable for administration of a precise amount. Pharmaceutical compositions are generally sterile.

### Methods of Transduction

The disclosure provides methods for transducing a recombinant viral vector. In several embodiments, the recombinant viral vector is administered to a subject in a dosing regimen that includes at least two doses (e.g., 2, 3, 4, 5, 6, 7, 8, 9, 10, 20, 30, 40, 50, 60, 70, 80, 90, 100, or more doses). Such methods generally include administering to the subject a recombinant viral vector (e.g., a parvoviral vector (e.g., an rAAV vector or a bocavirus vector), an adenoviral vector, or a retroviral vector) and/or an effective amount of an immunosuppressive regimen. In some embodiments, the immunosuppressive regimen includes one or more (e.g., 1, 2, 3, 4, 5, 6, 7, 8, 9, or 10) of a calcineurin inhibitor, a glucocorticoid, an antimetabolite, a mammalian target of rapamycin (mTOR) inhibitor, an alkylating agent, a purine biosynthesis inhibitor, an anti-cluster of differentiation 20 (CD20) antibody, a polyclonal anti-lymphocyte antibody, an immunomodulatory drug, or an immunoglobulin protease. In other embodiments, the method of transducing a recombinant viral vector includes administering the vector to a subject, in a dosing regimen including at least two doses, and administering an effective amount of an immunosuppressive regimen comprising one or more of fingolimod and an immunoglobulin protease.

In some embodiments, the transduction of the recombinant viral vector is improved relative to the transduction level achieved by administration of the recombinant viral vector to the subject in the dosing regimen in the absence of administration of the immunosuppressive regimen. In some embodiments, the immunosuppressive regimen improves transduction by inhibiting an immune response in the subject against the viral vector. In some embodiments, the inhibited immune an innate immune response, a B-cell mediated immune response, and/or a T-cell mediated immune response. In some embodiments, the innate immune response is a B-cell mediated immune response. In some embodiments, the innate immune response is a T-cell mediated immune response. In some embodiments, the immunosuppressive regimen improves viral uptake or improves transduction efficiency of the viral vector.

### Transgene Expression

The disclosure provides methods for improving transgene expression from a recombinant viral vector. In several embodiments, the recombinant viral vector is administered to a subject in a dosing regimen that includes at least two doses (e.g., 2, 3, 4, 5, 6, 7, 8, 9, 10, 20, 30, 40, 50, 60, 70, 80, 90, 100, or more doses). Such methods generally include administering to the subject a recombinant viral vector (e.g., a parvoviral vector (e.g., an rAAV vector or a bocavirus vector), an adenoviral vector, or a retroviral vector) and/or an effective amount of an immunosuppressive regimen. In some embodiments, the immunosuppressive regimen includes one or more (e.g., 1, 2, 3, 4, 5, 6, 7, 8, 9, or 10) of a calcineurin inhibitor, a glucocorticoid, an antimetabolite, a mammalian target of rapamycin (mTOR) inhibitor, an alkylating agent, a purine biosynthesis inhibitor, an anti-cluster of differentiation 20 (CD20) antibody, a polyclonal anti-lymphocyte antibody, an immunomodulatory drug, or an immunoglobulin protease. In some embodiments, the method of improving transgene expression includes administering to a subject a recombinant viral vector in a dosing regimen comprising at least two doses and an effective amount of an immunosuppressive regimen comprising one or more of fingolimod and an immunoglobulin protease.

In some embodiments, transgene expression from the recombinant viral vector is improved relative to the level of transgene expression achieved by administration of the recombinant viral vector to the subject in the dosing regimen in the absence of administration of the immunosuppressive regimen. In some embodiments, the transgene expression is improved by about 10-fold, about 20-fold, about 30-fold, about 40-fold, about 50-fold, about 60-fold, about 70-fold, about 80-fold, or about 90-fold relative to the level of transgene expression achieved by administration of the recombinant viral vector to the subject in the dosing regimen in the absence of administration of the immunosuppressive regimen.

### Reducing Neutralizing Antibodies

The disclosure provides methods for reducing the titer of neutralizing antibodies that bind to a recombinant viral vector. In several embodiments, the recombinant viral vector is administered to a subject in a dosing regimen that includes at least two doses (e.g., 2, 3, 4, 5, 6, 7, 8, 9, 10, 20, 30, 40, 50, 60, 70, 80, 90, 100, or more doses). Such methods generally include administering to the subject a recombinant viral vector (e.g., a parvoviral vector (e.g., an rAAV vector or a bocavirus vector), an adenoviral vector, or a retroviral vector) and/or an effective amount of an immunosuppressive regimen. In some embodiments, the immunosuppressive regimen includes one or more (e.g., 1, 2, 3, 4, 5, 6, 7, 8, 9, or 10) of a calcineurin inhibitor, a glucocorticoid, an antimetabolite, a mammalian target of rapamycin (mTOR) inhibitor, an alkylating agent, a purine biosynthesis inhibitor, an anti-cluster of differentiation 20 (CD20) antibody, a polyclonal anti-lymphocyte antibody, an immunomodulatory drug, or an immunoglobulin protease. In some embodiments, the method of reducing the titer of neutralizing antibodies that bind to a recombinant viral vector includes administering to a subject a recombinant viral vector in a dosing regimen comprising at least two doses and an effective amount of an immunosuppressive regimen comprising one or more of fingolimod and an immunoglobulin protease.

In some embodiments, the titer of neutralizing antibodies is reduced relative to the level of neutralizing antibodies resulting from administration of the recombinant viral vector to the subject in the dosing regimen in the absence of administration of the immunosuppressive regimen. In some embodiments, the titer of neutralizing antibodies is reduced about 2-fold, about 3-fold, about 4-fold, or about 5-fold relative to the level of neutralizing antibodies resulting from administration of the recombinant viral vector to the subject in the dosing regimen in the absence of administration of the immunosuppressive regimen.

### Methods of Treatment

Any of the methods disclosed herein can be used in the context of treating a disorder in a subject in need thereof.

The disclosure provides methods of treating a subject suffering from a genetic disease (e.g., cystic fibrosis, AAT deficiency, SP-B deficiency, and SP-C deficiency), an acquired pulmonary disease (e.g., chronic obstructive pulmonary disorder (COPD)), or an infectious disease (e.g., COVID-19). In some embodiments, the genetic disease is cystic fibrosis (CF), AAT deficiency, SP-B deficiency, or SP-C deficiency. In some embodiments, the genetic disease is CF. In some embodiments, the acquired pulmonary disease is COPD. In some embodiments, the infectious disease is a viral infection. In some embodiments, the viral infection is COVID-19.

For example, the disclosure provides a method of treating a disorder (e.g., a genetic disease (e.g., cystic fibrosis, AAT deficiency, SP-B deficiency, and SP-C deficiency), an acquired pulmonary disease (e.g., COPD), or an infectious disease (e.g., COVID-19)) in a subject in need thereof that includes administering a recombinant viral vector to the subject; and administering an effective amount of an immunosuppressive regimen including one or more (e.g., 1, 2, 3, 4, 5, 6, 7, 8, 9, or 10) of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease to the subject.

For example, in one aspect the disclosure provides a method of treating CF in a subject in need that includes administering an effective amount of an immunosuppressive regimen including one or more (e.g., 1, 2, or 3) of a calcineurin inhibitor, a glucocorticoid, and an antimetabolite to the subject; and administering at least a first dose and a second dose of rAAV including (i) an AV.TL65 capsid protein; and (ii) a polynucleotide comprising an F5 enhancer and a tg83 promoter operably linked to a CFTRΔR minigene.

In some embodiments, the disclosure provides an rAAV for use in treating cystic fibrosis in a subject in need thereof, the rAAV including (i) an AV.TL65 capsid protein; and (ii) a polynucleotide including an F5 enhancer and a tg83 promoter operably linked to a CFTRΔR minigene. In some embodiments, the rAAV is for use in combination with one or more additional therapeutic agents. The rAAV may include any of the polynucleotides described herein.

Any of the methods disclosed herein may involve administering an immunosuppressive regimen to a patient who has not yet been administered a recombinant viral vector. In other aspects, the methods may involve administering an immunosuppressive regimen concurrently with a recombinant viral vector. In yet other aspects, the methods may involve administering an immunosuppressive regimen following administration of a recombinant viral vector.

Compositions described herein (e.g., recombinant viral vectors, immunosuppressive agents, or pharmaceutical compositions) may be used in vivo as well as ex vivo. In vivo administration comprises administering the vectors of this disclosure directly to a subject. Pharmaceutical compositions can be supplied as liquid solutions or suspensions, as emulsions, or as solid forms suitable for dissolution or suspension in liquid prior to use. For administration into the respiratory tract, one exemplary mode of administration is by aerosol, using a composition that provides either a solid or liquid aerosol when used with an appropriate aerosolubilizer device. Another some mode of administration into the respiratory tract is using a flexible fiberoptic bronchoscope to instill the vectors.

A composition described herein (e.g., a recombinant viral vector, an immunosuppressive agent, or a pharmaceutical composition) can be administered by any suitable route, e.g., by inhalation, nebulization, aerosolization, intranasally, intratracheally, intrabronchially, orally, parenterally (e.g., intravenously, subcutaneously, or intramuscularly), orally, nasally, rectally, topically, or buccally. Such compositions can also be administered locally or systemically. In some embodiments, a composition described herein is administered in aerosolized particles intratracheally and/or intrabronchially using an atomizer sprayer (e.g., with a MADgic® laryngo-tracheal mucosal atomization device). In some embodiments, the composition is administered parentally. In other some embodiments, the composition is administered systemically. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition, and dosage ranges of vector solutions are within the skill of the art.

For administration to the upper (nasal) or lower respiratory tract by inhalation, the compositions described herein (e.g., recombinant viral vectors, immunosuppressive agents, or pharmaceutical compositions) are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the agent and a suitable powder base such as lactose or starch. The powder composition may be presented in unit dosage form in, for example, capsules or cartridges, or, e.g., gelatine or blister packs from which the powder may be administered with the aid of an inhalator, insufflator, or a metered-dose inhaler.

For intra-nasal administration, the agent may be administered via nose drops, a liquid spray, such as via a plastic bottle atomizer or metered-dose inhaler. Exemplary atomizers include the Mistometer (Vintrop) and the Medihaler (Riker).

Administration of the compositions described herein (e.g., recombinant viral vectors, immunosuppressive agents, or pharmaceutical compositions) may be continuous or intermittent, depending, for example, upon the recipient's physiological condition, whether the purpose of the administration is therapeutic or prophylactic, and other factors known to skilled practitioners. The compositions described herein can be administered at least twice, or more times (e.g., 2, 3, 4, 5, 6, 7, 8, 9, 10, 20, or more times), at the same or at different sites. The administration of the agents of the disclosure may be essentially continuous over a preselected period of time or may be in a series of spaced doses.

The dosing regimen of any one of the recombinant viral vectors described herein may include a first dose and a second dose of the recombinant viral vector. The second dose of the recombinant viral vector may be administered any suitable amount of time following the first dose, e.g., minutes, hours, days, weeks, months, or even years following the first dose. For example, the second dose of the recombinant viral vector may be administered to the subject at least about 1, 2, 3, or 4 weeks after the first dose. For example, the second dose of the recombinant viral vector may be administered to the subject about 4 weeks, about 2 months, about 6 months, or about 12 months after the first dose. In some embodiments, the second dose of the recombinant viral vector is administered to the subject about 4 weeks after the first dose.

The recombinant viral vector may be administered by inhalation, nebulization, aerosolization, intranasally, intratracheally, intrabronchially, orally, intravenously, subcutaneously, or intramuscularly. In some embodiments, the recombinant viral vector is administered by inhalation, nebulization, aerosolization, intranasally, intratracheally, and/or intrabronchially.

The dosing regimen of the immunosuppressive regimen includes at least a first dose. The first dose of the immunosuppressive regimen may be administered to the subject at any suitable time point relative to administration of the recombinant viral vector. For example, the first dose of the immunosuppressive regimen may be administered to the subject about 1 day, about 2 days, about 3 days, about 4 days, about 5 days, about 6 days, about 7 days, about 2 weeks, about 4 weeks, or about 8 weeks prior to the first dose of the dosing regimen of the recombinant viral vector. In some embodiments, the first dose of the immunosuppressive regimen is administered to the subject about 2 days prior to the first dose of the dosing regimen of the recombinant viral vector. In some embodiments, the first dose of the immunosuppressive regimen is administered to the subject about 1 day, about 2 days, about 3 days, about 4 days, about 5 days, about 6 days, about 7 days, about 2 weeks, about 3 weeks, about 4 weeks, or about 8 weeks after the first dose of the dosing regimen of the recombinant viral vector. In some embodiments, the first dose of the immunosuppressive regimen is administered to the subject about 7 days after the first dose of the dosing regimen of the recombinant viral vector. The immunosuppressive regimen may be administered to the subject every day, every two days, every three days, every four days, every five days, every six days, every week, every two weeks, every three weeks, every four weeks, every five weeks, every six weeks, every seven weeks, or every eight weeks. In some embodiments, the immunosuppressive regimen is administered to the subject every day.

The immunosuppressive regimen (e.g., one or more of a calcineurin inhibitor, a glucocorticoid, an antimetabolite, mTOR inhibitor, an alkylating agent, a purine biosynthesis inhibitor, CD20 antibody, a polyclonal anti-lymphocyte antibody, an immunomodulatory drug, or an immunoglobulin protease) may be administered to the subject intraperitoneally, orally, by inhalation, nebulization, aerosolization, intranasally, intratracheally, intrabronchially, intravenously, subcutaneously, or intramuscularly. In some embodiments, the calcineurin inhibitor is administered intraperitoneally. In some embodiments, the glucocorticoid is administered intraperitoneally. In some embodiments, the antimetabolite is administered orally.

The dosage of the present compositions will vary with the form of administration, the particular compound chosen and the physiological characteristics of the particular patient under treatment. It is desirable that the lowest effective concentration of virus be utilized in order to reduce the risk of undesirable effects, such as toxicity.

### Augmenters

The recombinant viral vectors (e.g., rAAVs) described herein can be used in combination with augmenters of transduction to achieve significant increases in transduction and/or expression of transgenes. Any suitable augmenter can be used. For example, U.S. Pat. No. 7,749,491, which is incorporated by reference herein in its entirety, describes suitable augmenters. The augmenter may be a proteasome modulating agent. The proteasome modulating agent may be an anthracycline (e.g., doxorubicin, idarubicin, aclarubicin, daunorubicin, epirubicin, valrubicin, or mitoxantrone), a proteasome inhibitor (e.g., bortezomib, carfilzomib, and ixazomib), a tripeptidyl aldehyde (e.g., N-acetyl-l-leucyl-l-leucyl-l-norieucine (LLnL)), or a combination thereof. In some embodiments, the augmenter is doxorubicin. In other embodiments, the augmenter is idarubicin.

The rAAV and the augmenter(s) may be contacted with a cell, or administered to a subject, in the same composition or in different compositions (e.g., pharmaceutical compositions). The contacting or the administration of the rAAV and the augmenter(s) may be sequential (e.g., rAAV followed by the augmenter(s), or vice versa) or simultaneous.

In some embodiments, any of the immunosuppressive agents disclosed herein may function as an augmenter.

### EXEMPLARY EMBODIMENTS

In one embodiment, a method of inhibiting suppression of, e.g., expression, of a recombinant viral vector administered to a mammal is provided. In one embodiment the method includes administering to a mammal the recombinant viral vector, e.g., at least two doses, and an effective amount of an immunosuppressive regimen comprising two or more of a calcineurin inhibitor, a glucocorticoid, an antimetabolite, a mammalian target of rapamycin (mTOR) inhibitor, an alkylating agent, a purine biosynthesis inhibitor, an anti-cluster of differentiation 20 (CD20) antibody, a polyclonal anti-lymphocyte antibody, an immunomodulatory drug, or an immunoglobulin protease. In one embodiment, the method comprises administering to the mammal at least two doses of the recombinant viral vector and an effective amount of an immunosuppressive regimen comprising one or more of fingolimod and an immunoglobulin protease. In one embodiment, the transduction of the recombinant viral vector is improved relative to the transduction level achieved by administration of the recombinant viral vector to the mammal in the dosing regimen in the absence of administration of the immunosuppressive regimen. In one embodiment, the immunosuppressive regimen improves transduction by inhibiting an immune response in the subject against the viral vector. In one embodiment, the immune response is an innate immune response, a B-cell mediated immune response, and/or a T-cell mediated immune response. In one embodiment, the immunosuppressive regimen improves viral uptake or improves transduction efficiency of the viral vector.

In one embodiment, the method includes administering to a mammal, administered at least two doses of a recombinant viral vector, an effective amount of an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease. In one embodiment, a method of improving transgene expression from a recombinant viral vector in a mammal is provided, where the vector is administered to the mammal in a dosing regimen comprising at least two doses, comprising administering to the mammal an effective amount of an immunosuppressive regimen comprising one or more of fingolimod and an immunoglobulin protease. In one embodiment, transgene expression from the recombinant viral vector is improved relative to the level of transgene expression achieved by administration of the recombinant viral vector to the mammal in the dosing regimen in the absence of administration of the immunosuppressive regimen. In one embodiment, transgene expression is improved by about 10-fold, about 20-fold, about 30-fold, about 40-fold, about 50-fold, about 80-fold, about 70-fold, about 80-fold, or about 90-fold relative to the level of transgene expression achieved by administration of the recombinant viral vector to the mammal in the dosing regimen in the absence of administration of the immunosuppressive regimen.

Also provided is a method of reducing the titer of neutralizing antibodies that bind to a recombinant viral vector administered to a mammal in a dosing regimen comprising at least two doses, comprising administering to the mammal an effective amount of an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease. In one embodiment, the method includes administering to the mammal an effective amount of an immunosuppressive regimen comprising one or more of fingolimod and an immunoglobulin protease. In one embodiment, the titer of neutralizing antibodies is reduced relative to the level of neutralizing antibodies resulting from administration of the recombinant viral vector to the mammal in the dosing regimen in the absence of administration of the immunosuppressive regimen. In one embodiment, the titer of neutralizing antibodies is reduced about 2-fold, about 3-fold, about 4-fold, or about 5-fold relative to the level of neutralizing antibodies resulting from administration of the recombinant viral vector to the mammal in the dosing regimen in the absence of administration of the immunosuppressive regimen.

In one embodiment, the immunosuppressive regimen comprises two or more of a calcineurin inhibitor, a glucocorticoid, and an antimetabolite. In one embodiment, the immunosuppressive regimen comprises a calcineurin inhibitor, a glucocorticoid, and an antimetabolite. In one embodiment, the calcineurin inhibitor is cyclosporine, tacrolimus, or a combination thereof. In one embodiment, the calcineurin inhibitor is cyclosporine. In one embodiment, the glucocorticoid is methylprednisolone, prednisolone, hydrocortisone, dexamethasone, cortisone, budesonide, betamethasone, beclomethasone, triamcinolone, or a combination thereof. In one embodiment, the glucocorticoid is methylprednisolone. In one embodiment, the antimetabolite is a purine analogue, a pyrimidine analogue, a nucleoside analogue, a nucleotide analogue, an antifolate, or a combination thereof. In one embodiment, the purine analogue is azathioprine, mercaptopurine, clofarabine, a thiopurine, fludarabine, pentostatin, cladribine, or a combination thereof. In one embodiment, the purine analogue is azathioprine. In one embodiment, the mTOR inhibitor is rapamycin, everolimus, temsirolimus, ridaforolimus, or a combination thereof. In one embodiment, the alkylating agent is cyclophosphamide. In one embodiment, the purine biosynthesis inhibitor is mycophenolate mofetil (MMF), mycophenolate sodium, or a combination thereof. In one embodiment, the anti-CD20 antibody is rituximab. In one embodiment, the polyclonal anti-lymphocyte antibody is anti-thymocyte globulin (ATG). In one embodiment, the immunomodulatory drug is fingolimod. In one embodiment, the dosing regimen of the recombinant viral vector comprises at least a first dose and a second dose of the recombinant viral vector. In one embodiment, the second dose of the recombinant viral vector is administered to the subject at least about 4 weeks after the first dose. In one embodiment, the second dose of the recombinant viral vector is administered to the subject about 4 weeks, about 2 months, about 6 months, or about 12 months after the first dose. In one embodiment, the second dose of the recombinant viral vector is administered to the subject about 4 weeks after the first dose. In one embodiment, the immunosuppressive regimen comprises at least a first dose. In one embodiment, the first dose of the immunosuppressive regimen is administered to the subject about 1 day, about 2 days, about 3 days, about 4 days, about 5 days, about 6 days, about 7 days, about 2 weeks, about 4 weeks, or about 8 weeks prior to the first dose of the dosing regimen of the recombinant viral vector. In one embodiment, the first dose of the immunosuppressive regimen is administered to the subject about 2 days prior to the first dose of the dosing regimen of the recombinant viral vector. In one embodiment, the first dose of the immunosuppressive regimen is administered to the subject on the same day as the first dose of the dosing regimen of the recombinant viral vector. In one embodiment, the first dose of the immunosuppressive regimen is administered to the subject about 1 day, about 2 days, about 3 days, about 4 days, about 5 days, about 6 days, about 7 days, about 2 weeks, about 3 weeks, about 4 weeks, or about 8 weeks after the first dose of the dosing regimen of the recombinant viral vector. In one embodiment, the first dose of the immunosuppressive regimen is administered to the subject about 7 days after the first dose of the dosing regimen of the recombinant viral vector. In one embodiment, the immunosuppressive regimen is administered to the subject every day, every two days, every three days, every four days, every five days, every six days, every week, every two weeks, every three weeks, every four weeks, every five weeks, every six weeks, every seven weeks, or every eight weeks. In one embodiment, the immunosuppressive regimen is administered to the subject every day. In one embodiment, wherein the recombinant viral vector is a recombinant parvoviral vector, a recombinant retroviral vector, or a recombinant adenoviral vector. In one embodiment, the recombinant viral vector is a recombinant parvoviral vector. In one embodiment, the parvoviral vector is a recombinant adeno-associated virus (rAAV) or a recombinant bocavirus vector. In one embodiment, the parvoviral vector is an rAAV. In one embodiment, the recombinant parvoviral vector comprises a capsid protein and a polynucleotide comprising an enhancer and/or a promoter operably linked to a transgene. In one embodiment, the capsid protein comprises an AV.TL65 capsid protein, an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AVrh.10 capsid protein, a bocavirus capsid protein, a variant thereof, or a combination thereof. In one embodiment, the capsid protein is an AV.TL65 capsid protein or a variant thereof. In one embodiment, the capsid protein is a bocavirus capsid protein. In one embodiment, the capsid protein is a human bocavirus (HBoV) capsid protein. In one embodiment, the human bocavirus capsid protein is an HBoV1 capsid protein, an HBoV2 capsid protein, an HBoV3 capsid protein, or an HBoV4 capsid protein. In one embodiment, the enhancer comprises an F5 enhancer or a variant thereof. In one embodiment, the promoter comprises a tg83 promoter or a variant thereof. In one embodiment, the transgene is a therapeutic protein. In one embodiment, the therapeutic protein is a CFTRΔR minigene or a variant thereof. In one embodiment, the therapeutic protein is alpha-1 antitrypsin (AAT), surfactant protein (SP)-B, SP-C, or a variant thereof. In one embodiment, the recombinant parvoviral vector is an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof. In one embodiment, the AV.TL65 capsid protein comprises the amino acid sequence of SEQ ID NO:13 or the variant comprises a sequence having at least 80% sequence identity to SEQ ID NO:13. In one embodiment, the F5 enhancer comprises the polynucleotide sequence of SEQ ID NO:1 or the variant comprises a sequence having at least 80% sequence identity to SEQ ID NO:1. In one embodiment, the F5 enhancer comprises the polynucleotide sequence of SEQ ID NO:14 or the variant comprises a sequence having at least 80% sequence identity to SEQ ID NO:14. In one embodiment, the tg83 promoter comprises the polynucleotide sequence of SEQ ID NO:2 or the variant comprises a sequence having at least 80% sequence identity to SEQ ID NO:2. In one embodiment, the CFTRΔR minigene is a human CFTRΔR minigene. In one embodiment, the human CFTRΔR minigene is encoded by a polynucleotide comprising the sequence of SEQ ID NO:4 or a variant thereof comprising a sequence having at least 80% sequence identity to SEQ ID NO:4. In one embodiment, the polynucleotide comprises, in a 5′-to-3′ direction, the F5 enhancer, the tg83 promoter, and the CFTRΔR minigene. In one embodiment, the recombinant retroviral vector is a recombinant lentiviral vector. In one embodiment, the mammal is suffering from a genetic disease, an acquired pulmonary disease, or an infectious disease. In one embodiment, the genetic disease is cystic fibrosis, AAT deficiency, SP-B deficiency, or SP-C deficiency. In one embodiment, the genetic disease is cystic fibrosis. In one embodiment, the acquired pulmonary disease is chronic obstructive pulmonary disorder (COPD). In one embodiment, the infectious disease is a viral infection. In one embodiment, the viral infection is COVID-19. In one embodiment, the method further comprises administering one or more additional therapeutic agents to the mammal. In one embodiment, the one or more additional therapeutic agents includes an augmenter, an antibiotic, a mucus thinner, a CFTR modulator, a mucolytic, normal saline, hypertonic saline, an immunosuppressive agent, or a combination thereof. In one embodiment, the augmenter comprises an anthracycline, a proteasome inhibitor, a tripeptidyl aldehyde, or a combination thereof. In one embodiment, the anthracycline comprises doxorubicin, idarubicin, aclarubicin, daunorubicin, epirubicin, calrubicin, mitoxantrone, or a combination thereof. In one embodiment, the proteasome inhibitor comprises bortezomib (VELCADE®), carfilzomib, ixazomib, or a combination thereof. In one embodiment, the recombinant viral vector is administered by inhalation, nebulization, aerosolization, intranasally, intratracheally, intrabronchially, orally, intravenously, subcutaneously, or intramuscularly. In one embodiment, the recombinant viral vector is administered by inhalation, nebulization, aerosolization, intranasally, intratracheally, and/or intrabronchially. In one embodiment, the immunosuppressive regimen is administered intraperitoneally, orally, by inhalation, nebulization, aerosolization, intranasally, intratracheally, intrabronchially, intravenously, subcutaneously, or intramuscularly. In one embodiment, the calcineurin inhibitor is administered intraperitoneally. In one embodiment, the glucocorticoid is administered intraperitoneally. In one embodiment, the antimetabolite is administered orally. In one embodiment, the mammal is a human.

Further provided is a method of administering a recombinant viral vector to a subject, comprising: administering an effective amount of an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease to the subject; and administering a recombinant viral vector to the subject.

In one embodiment, a method of treating cystic fibrosis in a subject in need thereof is provided, comprising: administering an effective amount of an immunosuppressive regimen comprising two or more of a calcineurin inhibitor, a glucocorticoid, and an antimetabolite to the subject; and administering at least a first dose and a second dose of rAAV comprising (i) an AV.TL65 capsid protein; and (ii) a polynucleotide comprising an F5 enhancer and a tg83 promoter operably linked to a CFTRΔR minigene.

The disclosure provides an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in improving transduction of a recombinant viral vector that is administered to a mammal in a dosing regimen comprising at least two doses. Also provided is an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in improving transgene expression from a recombinant viral vector that is administered to a mammal in a dosing regimen comprising at least two doses.

Further provided is an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in reducing the titer of neutralizing antibodies that bind to a recombinant viral vector that is administered to a mammal in a dosing regimen comprising at least two doses. In one embodiment, an immunosuppressive regimen is provided comprising two or more of a calcineurin inhibitor, a glucocorticoid, and an antimetabolite for use in treating cystic fibrosis in a mammal in need thereof, wherein the immunosuppressive regimen is administered to the subject in combination with at least a first dose and a second dose of rAAV comprising (i) an AV.TL65 capsid protein; and (ii) a polynucleotide comprising an F5 enhancer and a tg83 promoter operably linked to a CFTRΔR minigene. In one embodiment, an immunosuppressive regimen is provided comprising one or more of fingolimod and an immunoglobulin protease for use in (i) improving transduction of a recombinant viral vector that is administered to a mammal in a dosing regimen comprising at least two doses; (ii) improving transgene expression from a recombinant viral vector that is administered to a mammal in a dosing regimen comprising at least two doses; and/or (iii) reducing the titer of neutralizing antibodies that bind to a recombinant viral vector that is administered to a mammal in a dosing regimen comprising at least two doses.

Also provided is a kit comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in (i) improving transduction of a recombinant viral vector that is administered to a mammal in a dosing regimen comprising at least two doses; (ii) improving transgene expression from a recombinant viral vector that is administered to a mammal in a dosing regimen comprising at least two doses; and/or (iii) reducing the titer of neutralizing antibodies that bind to a recombinant viral vector that is administered to a mammal in a dosing regimen comprising at least two doses. In one embodiment, a kit is provided comprising one or more of fingolimod and an immunoglobulin protease for use in (i) improving transduction of a recombinant viral vector that is administered to a mammal in a dosing regimen comprising at least two doses; (ii) improving transgene expression from a recombinant viral vector that is administered to a mammal in a dosing regimen comprising at least two doses; and/or (iii) reducing the titer of neutralizing antibodies that bind to a recombinant viral vector that is administered to a mammal in a dosing regimen comprising at least two doses.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Administration of Immunosuppressants Improves the Transduction of AAV In Vivo

Previous studies showed that repeat administration of an adeno-associated viral vector (rAAV2.5T) to ferret lungs led to poorer outcomes with respect to transgene expression and the production of neutralizing antibodies (NAbs) compared to single-dose administration. In the current study, an immune suppression strategy was tested for its effects on rAAV2.5T repeat dosing with respect to transgene expression. This immunosuppressant (IS) regimen involved daily administration over the course of one month of cyclosporine (1 mg/kg) administered intraperitoneally, methylprednisolone (2 mg/kg) administered intraperitoneally, and azathioprine (2 mg/kg) administered orally. The results showed that this triple IS strategy was an effective means of both improving transgene expression and reducing NAb levels following a second administration of aerosolized rAAV2.5T to ferret lungs.

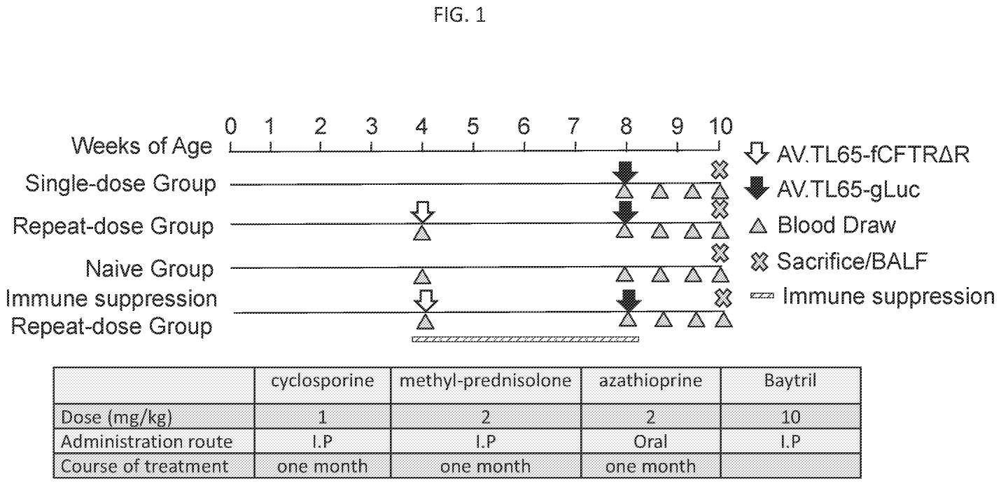

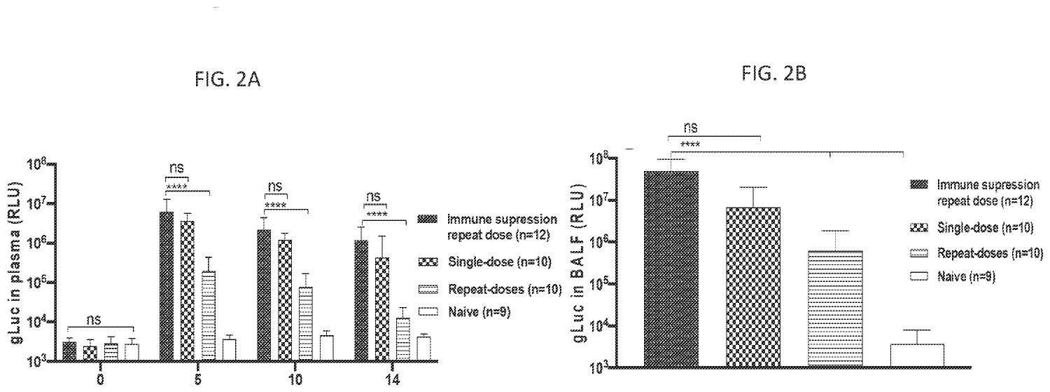

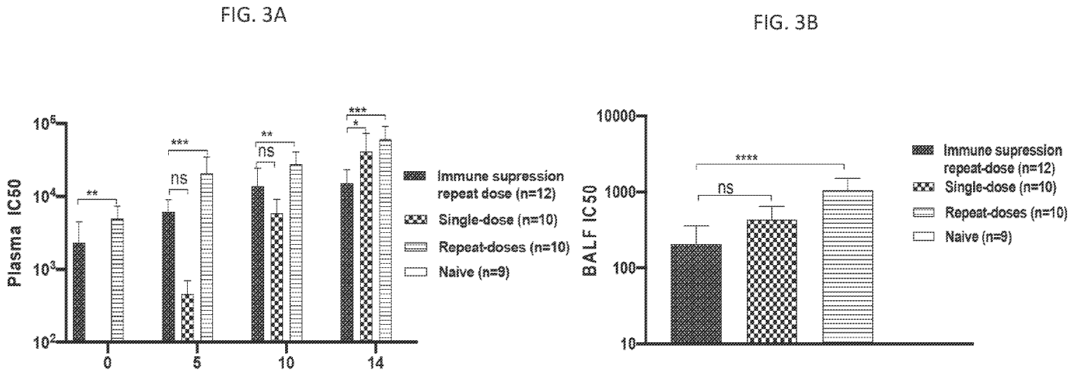

Ferret lungs were dosed with AAV2.5T-fCFTRΔR (ferret CFTRΔR) at 4 weeks of age and with a reporter vector (AAV2.5T-gLuc, gaussia luciferase) at 8 weeks of age (FIG. 1). Transgene expression (secreted gLuc) was monitored in the blood and bronchoalveolar lavage fluid (BALF) at 14 days post-infection. The immuno-suppressed group received daily administration of IS for 30 days starting at 2 days prior to the first vector dose. The IS regimen led to gLuc concentrations in the plasma and BALF that were 90- and 78.6-fold higher than the repeat-dose group without IS as shown in FIGS. 2A and 2B, respectively. Notably, gLuc expression in the BALF of the repeat-dose IS group was 6.2-fold higher than in ferrets receiving just the reporter vector at the same age (8 weeks), suggesting there may be a component of innate immunity that limits vector transduction. The IS strategy did not lead to complete elimination of the humoral immunity elicited by AAV2.5T, with substantial increases in NAbs detected in the plasma of ferrets of the IS group following administration of the second vector. However, the NAbs titers in the plasma and the BALF were lower in the repeat-dose group treated with IS (4-fold and 5.1-fold, respectively) than those in the repeat-dose group without IS at 14-day post-delivery of the second vector as shown in FIGS. 3A and 3B. Importantly, these preexisting NAbs did not inhibit repeat-dose transduction in ferrets treated with IS, suggesting the IS regimen may inhibit the formation of high-affinity NAbs.

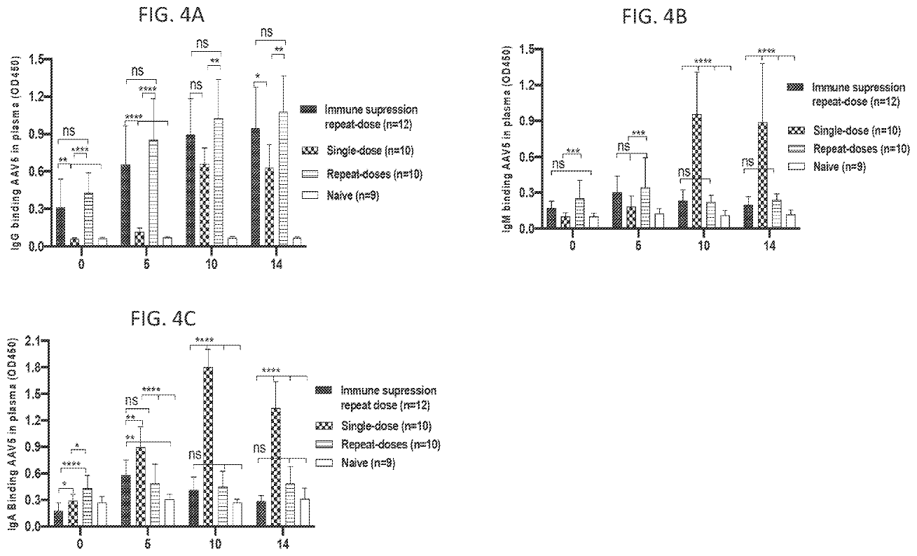

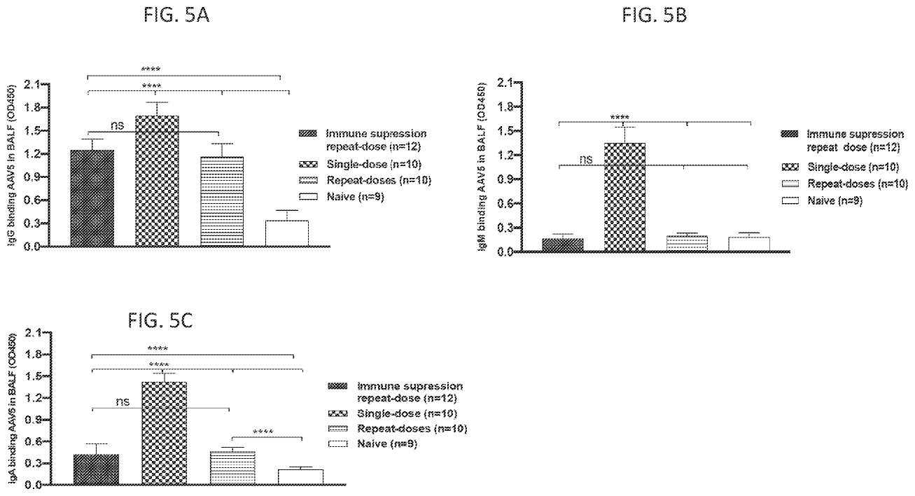

This study also revealed that there was no significant difference in the levels of AAV5 capsid-specific immunoglobulins (IgG, IgM, and IgA) in the BAFL between the two dosing groups with or without IS (FIGS. 5A-5C). Similarly, the levels of IgG, IgM and IgA in plasma did not differ significantly between the two groups (FIGS. 4A-4C). Collectively, the data show that the three immunosuppressants tested improved transgene expression after repeat AAV2.5T dosing, and that this correlated with an inhibition, though not elimination, of the humoral immune response. The results also suggest that the administration of immunosuppressants can facilitate gene therapy with repeat-dose regimens by improving transduction, by improving transgene expression, and/or by reducing the titer of NAbs.

SEQUENCE LISTING

| SEQ | | |
|---|---|---|
| ID NO | Name | Sequence |
| 1 | F5 | GAATTCGTGGTGAGCGTCTGGGCATGTCTGGGCATGTCTGGGCATGTC |
| | Enhancer | TGGGCATGTCGGGCATTCTGGGCGTCTGGGCATGTCTGGGCATGTCTG |
| | with 5′ | GGCATCTCGAG |
| | EcoRI and | |
| | 3′XhoI | |
| | sites | |
| 2 | tg83 | AACGGTGACGTGCACGCGTGGGCGGAGCCATCACGCAGGTTGCTATA |
| | Promoter | TAAGCAGAGCTCGTTTAGTGAACCGTCAGA |
| 2 | 5′-UTR | GTCGAGCCCGAGAGACC |
| 4 | hCFTRΔR | ATGCAGAGGTCGCCTCTGGAAAAGGCCAGCGTTGTCTCCAAACTTTTTT |
| | | TCAGCTGGACCAGACCAATTTTGAGGAAAGGATACAGACAGCGCCTGG |
| | | AATTGTCAGACATATACCAAATCCCTTCTGTTGATTCTGCTGACAATCTA |
| | | TCTGAAAAATTGGAAAGAGAATGGGATAGAGAGCTGGCTTCAAAGAAAA |
| | | ATCCTAAACTCATTAATGCCCTTCGGCGATGTTTTTTCTGGAGATTTATG |
| | | TTCTATGGAATCTTTTTATATTTAGGGGAAGTCACCAAAGCAGTACAGC |
| | | CTCTCTTACTGGGAAGAATCATAGCTTCCTATGACCCGGATAACAAGGA |
| | | GGAACGCTCTATCGCGATTTATCTAGGCATAGGCTTATGCCTTCTCTTT |
| | | ATTGTGAGGACACTGCTCCTACACCCAGCCATTTTTGGCCTTCATCACA |
| | | TTGGAATGCAGATGAGAATAGCTATGTTTAGTTTGATTTATAAGAAGACT |
| | | TTAAAGCTGTCAAGCCGTGTTCTAGATAAAATAAGTATTGGACAACTTGT |
| | | TAGTCTCCTTTCCAACAACCTGAACAAATTTGATGAAGGACTTGCATTG |
| | | GCACATTTCGTGTGGATCGCTCCTTTGCAAGTGGCACTCCTCATGGGG |
| | | CTAATCTGGGAGTTGTTACAGGCGTCTGCCTTCTGTGGACTTGGTTTCC |
| | | TGATAGTCCTTGCCCTTTTTCAGGCTGGGCTAGGGAGAATGATGATGAA |
| | | GTACAGAGATCAGAGAGCTGGGAAGATCAGTGAAAGACTTGTGATTAC |
| | | CTCAGAAATGATCGAGAACATCCAATCTGTTAAGGCATACTGCTGGGAA |
| | | GAAGCAATGGAAAAAATGATTGAAAACTTAAGACAAACAGAACTGAAAC |
| | | TGACTCGGAAGGCAGCCTATGTGAGATACTTCAATAGCTCAGCCTTCTT |
| | | CTTCTCAGGGTTCTTTGTGGTGTTTTTATCTGTGCTTCCCTATGCACTAA |
| | | TCAAAGGAATCATCCTCCGGAAAATATTCACCACCATCTCATTCTGCATT |
| | | GTTCTGCGCATGGCGGTCACTCGGCAATTTCCCTGGGCTGTACAAACA |
| | | TGGTATGACTCTCTTGGAGCAATAAACAAAATACAGGATTTCTTACAAAA |
| | | GCAAGAATATAAGACATTGGAATATAACTTAACGACTACAGAAGTAGTG |
| | | ATGGAGAATGTAACAGCCTTCTGGGAGGAGGGATTTGGGGAATTATTT |
| | | GAGAAAGCAAAACAAAACAATAACAATAGAAAAACTTCTAATGGTGATG |
| | | ACAGCCTCTTCTTCAGTAATTTCTCACTTCTTGGTACTCCTGTCCTGAAA |
| | | GATATTAATTTCAAGATAGAAAGAGGACAGTTGTTGGCGGTTGCTGGAT |
| | | CCACTGGAGCAGGCAAGACTTCACTTCTAATGATGATTATGGGAGAACT |
| | | GGAGCCTTCAGAGGGTAAAATTAAGCACAGTGGAAGAATTTCATTCTGT |
| | | TCTCAGTTTTCCTGGATTATGCCTGGCACCATTAAAGAAAATATCATCTT |
| | | TGGTGTTTCCTATGATGAATATAGATACAGAAGCGTCATCAAAGCATGC |
| | | CAACTAGAAGAGGACATCTCCAAGTTTGCAGAGAAAGACAATATAGTTC |
| | | TTGGAGAAGGTGGAATCACACTGAGTGGAGGTCAACGAGCAAGAATTT |
| | | CTTTAGCAAGAGCAGTATACAAAGATGCTGATTTGTATTTATTAGACTCT |
| | | CCTTTTGGATACCTAGATGTTTTAACAGAAAAAGAAATATTTGAAAGCTG |
| | | TGTCTGTAAACTGATGGCTAACAAAACTAGGATTTTGGTCACTTCTAAAA |
| | | TGGAACATTTAAAGAAAGCTGACAAAATATTAATTTTGCATGAAGGTAGC |
| | | AGCTATTTTTATGGGACATTTTCAGAACTCCAAAATCTACAGCCAGACTT |
| | | TAGCTCAAAACTCATGGGATGTGATTCTTTCGACCAATTTAGTGCAGAA |
| | | AGAAGAAATTCAATCCTAACTGAGACCTTACACCGTTTCTCATTAGAAG |
| | | GAGATGCTCCTGTCTCCTGGACAGAAACAAAAAAACAATCTTTTAAACA |
| | | GACTGGAGAGTTTGGGGAAAAAAGGAAGAATTCTATTCTCAATCCAATC |
| | | AACTCTACGCTTCAGGCACGAAGGAGGCAGTCTGTCCTGAACCTGATG |
| | | ACACACTCAGTTAACCAAGGTCAGAACATTCACCGAAAGACAACAGCAT |
| | | CCACACGAAAAGTGTCACTGGCCCCTCAGGCAAACTTGACTGAACTGG |
| | | ATATATATTCAAGAAGGTTATCTCAAGAAACTGGCTTGGAAATAAGTGAA |
| | | GAAATTAACGAAGAAGACTTAAAGGAGTGCCTTTTTGATGATATGGAGA |
| | | GCATACCAGCAGTGACTACATGGAACACATACCTTCGATATATTACTGT |
| | | CCACAAGAGCTTAATTTTTGTGCTAATTTGGTGCTTAGTAATTTTTCTGG |
| | | CAGAGGTGGCTGCTTCTTTGGTTGTGCTGTGGCTCCTTGGAAACACTC |
| | | CTCTTCAAGACAAAGGGAATAGTACTCATAGTAGAAATAACAGCTATGC |
| | | AGTGATTATCACCAGCACCAGTTCGTATTATGTGTTTTACATTTACGTGG |
| | | GAGTAGCCGACACTTTGCTTGCTATGGGATTCTTCAGAGGTCTACCACT |
| | | GGTGCATACTCTAATCACAGTGTCGAAAATTTTACACCACAAAATGTTAC |
| | | ATTCTGTTCTTCAAGCACCTATGTCAACCCTCAACACGTTGAAAGCAGG |
| | | TGGGATTCTTAATAGATTCTCCAAAGATATAGCAATTTTGGATGACCTTC |
| | | TGCCTCTTACCATATTTGACTTCATCCAGTTGTTATTAATTGTGATTGGA |
| | | GCTATAGCAGTTGTCGCAGTTTTACAACCCTACATCTTTGTTGCAACAG |
| | | TGCCAGTGATAGTGGCTTTTATTATGTTGAGAGCATATTTCCTCCAAACC |
| | | TCACAGCAACTCAAACAACTGGAATCTGAAGGCAGGAGTCCAATTTTCA |
| | | CTCATCTTGTTACAAGCTTAAAAGGACTATGGACACTTCGTGCCTTCGG |
| | | ACGGCAGCCTTACTTTGAAACTCTGTTCCACAAAGCTCTGAATTTACATA |
| | | CTGCCAACTGGTTCTTGTACCTGTCAACACTGCGCTGGTTCCAAATGAG |
| | | AATAGAAATGATTTTTGTCATCTTCTTCATTGCTGTTACCTTCATTTCCAT |
| | | TTTAACAACAGGAGAAGGAGAAGGAAGAGTTGGTATTATCCTGACTTTA |
| | | GCCATGAATATCATGAGTACATTGCAGTGGGCTGTAAACTCCAGCATAG |
| | | ATGTGGATAGCTTGATGCGATCTGTGAGCCGAGTCTTTAAGTTCATTGA |
| | | CATGCCAACAGAAGGTAAACCTACCAAGTCAACCAAACCATACAAGAAT |
| | | GGCCAACTCTCGAAAGTTATGATTATTGAGAATTCACACGTGAAGAAAG |
| | | ATGACATCTGGCCCTCAGGGGGCCAAATGACTGTCAAAGATCTCACAG |
| | | CAAAATACACAGAAGGTGGAAATGCCATATTAGAGAACATTTCCTTCTC |
| | | AATAAGTCCTGGCCAGAGGGTGGGCCTCTTGGGAAGAACTGGATCAGG |
| | | GAAGAGTACTTTGTTATCAGCTTTTTTGAGACTACTGAACACTGAAGGA |
| | | GAAATCCAGATCGATGGTGTGTCTTGGGATTCAATAACTTTGCAACAGT |
| | | GGAGGAAAGCCTTTGGAGTGATACCACAGAAAGTATTTATTTTTTCTGG |
| | | AACATTTAGAAAAAACTTGGATCCCTATGAACAGTGGAGTGATCAAGAA |
| | | ATATGGAAAGTTGCAGATGAGGTTGGGCTCAGATCTGTGATAGAACAGT |
| | | TTCCTGGGAAGCTTGACTTTGTCCTTGTGGATGGGGGCTGTGTCCTAA |
| | | GCCATGGCCACAAGCAGTTGATGTGCTTGGCTAGATCTGTTCTCAGTAA |
| | | GGCGAAGATCTTGCTGCTTGATGAACCCAGTGCTCATTTGGATCCAGTA |
| | | ACATACCAAATAATTAGAAGAACTCTAAAACAAGCATTTGCTGATTGCAC |
| | | AGTAATTCTCTGTGAACACAGGATAGAAGCAATGCTGGAATGCCAACAA |
| | | TTTTTGGTCATAGAAGAGAACAAAGTGCGGCAGTACGATTCCATCCAGA |
| | | AACTGCTGAACGAGAGGAGCCTCTTCCGGCAAGCCATCAGCCCCTCCG |
| | | ACAGGGTGAAGCTCTTTCCCCACCGGAACTCAAGCAAGTGCAAGTCTA |
| | | AGCCCCAGATTGCTGCTCTGAAAGAGGAGACAGAAGAAGAGGTGCAAG |
| | | ATACAAGGCTTTAG |
| 5 | 3′-UTR | AGAGCAGCATAAATGTTGACATGGGACATTTGCTCATGGAATTGG |
| 6 | s-pA | AATAAAGAGCTCAGATGCATCGATCAGAGTGTGTTGGTTTTTTGTGTGT |
| | | A |
| 7 | F5 | GAATTCGTGGTGAGCGTCTGGGCATGTCTGGGCATGTCTGGGCATGTC |
| | Enhancer, | TGGGCATGTCGGGCATTCTGGGCGTCTGGGCATGTCTGGGCATGTCTG |
| | Tg83 | GGCATCTCGAGAACGGTGACGTGCACGCGTGGGCGGAGCCATCACGC |
| | Promoter. | AGGTTGCTATATAAGCAGAGCTCGTTTAGTGAACCGTCAGAGTCGAGC |
| | 5′-UTR, | CCGAGAGACCATGCAGAGGTCGCCTCTGGAAAAGGCCAGCGTTGTCTC |
| | hCFTRΔR | CAAACTTTTTTTCAGCTGGACCAGACCAATTTTGAGGAAAGGATACAGA |
| | | CAGCGCCTGGAATTGTCAGACATATACCAAATCCCTTCTGTTGATTCTG |
| | | CTGACAATCTATCTGAAAAATTGGAAAGAGAATGGGATAGAGAGCTGGC |
| | | TTCAAAGAAAAATCCTAAACTCATTAATGCCCTTCGGCGATGTTTTTTCT |
| | | GGAGATTTATGTTCTATGGAATCTTTTTATATTTAGGGGAAGTCACCAAA |
| | | GCAGTACAGCCTCTCTTACTGGGAAGAATCATAGCTTCCTATGACCCGG |
| | | ATAACAAGGAGGAACGCTCTATCGCGATTTATCTAGGCATAGGCTTATG |
| | | CCTTCTCTTTATTGTGAGGACACTGCTCCTACACCCAGCCATTTTTGGC |
| | | CTTCATCACATTGGAATGCAGATGAGAATAGCTATGTTTAGTTTGATTTA |
| | | TAAGAAGACTTTAAAGCTGTCAAGCCGTGTTCTAGATAAAATAAGTATTG |
| | | GACAACTTGTTAGTCTCCTTTCCAACAACCTGAACAAATTTGATGAAGG |
| | | ACTTGCATTGGCACATTTCGTGTGGATCGCTCCTTTGCAAGTGGCACTC |
| | | CTCATGGGGCTAATCTGGGAGTTGTTACAGGCGTCTGCCTTCTGTGGA |
| | | CTTGGTTTCCTGATAGTCCTTGCCCTTTTTCAGGCTGGGCTAGGGAGAA |
| | | TGATGATGAAGTACAGAGATCAGAGAGCTGGGAAGATCAGTGAAAGAC |
| | | TTGTGATTACCTCAGAAATGATCGAGAACATCCAATCTGTTAAGGCATA |
| | | CTGCTGGGAAGAAGCAATGGAAAAAATGATTGAAAACTTAAGACAAACA |
| | | GAACTGAAACTGACTCGGAAGGCAGCCTATGTGAGATACTTCAATAGCT |
| | | CAGCCTTCTTCTTCTCAGGGTTCTTTGTGGTGTTTTTATCTGTGCTTCCC |
| | | TATGCACTAATCAAAGGAATCATCCTCCGGAAAATATTCACCACCATCT |
| | | CATTCTGCATTGTTCTGCGCATGGCGGTCACTCGGCAATTTCCCTGGG |
| | | CTGTACAAACATGGTATGACTCTCTTGGAGCAATAAACAAAATACAGGA |
| | | TTTCTTACAAAAGCAAGAATATAAGACATTGGAATATAACTTAACGACTA |
| | | CAGAAGTAGTGATGGAGAATGTAACAGCCTTCTGGGAGGAGGGATTTG |
| | | GGGAATTATTTGAGAAAGCAAAACAAAACAATAACAATAGAAAAACTTCT |
| | | AATGGTGATGACAGCCTCTTCTTCAGTAATTTCTCACTTCTTGGTACTCC |
| | | TGTCCTGAAAGATATTAATTTCAAGATAGAAAGAGGACAGTTGTTGGCG |
| | | GTTGCTGGATCCACTGGAGCAGGCAAGACTTCACTTCTAATGATGATTA |
| | | TGGGAGAACTGGAGCCTTCAGAGGGTAAAATTAAGCACAGTGGAAGAA |
| | | TTTCATTCTGTTCTCAGTTTCCTGGATTATGCCTGGCACCATTAAAGAA |
| | | AATATCATCTTTGGTGTTTCCTATGATGAATATAGATACAGAAGCGTCAT |
| | | CAAAGCATGCCAACTAGAAGAGGACATCTCCAAGTTTGCAGAGAAAGA |
| | | CAATATAGTTCTTGGAGAAGGTGGAATCACACTGAGTGGAGGTCAACG |
| | | AGCAAGAATTTCTTTAGCAAGAGCAGTATACAAAGATGCTGATTTGTATT |
| | | TATTAGACTCTCCTTTTGGATACCTAGATGTTTTAACAGAAAAAGAAATA |
| | | TTTGAAAGCTGTGTCTGTAAACTGATGGCTAACAAAACTAGGATTGG |
| | | TCACTTCTAAAATGGAACATTTAAAGAAAGCTGACAAAATATTAATTG |
| | | CATGAAGGTAGCAGCTATTTTTATGGGACATTTTCAGAACTCCAAAATCT |
| | | ACAGCCAGACTTTAGCTCAAAACTCATGGGATGTGATTCTTTCGACCAA |
| | | TTTAGTGCAGAAAGAAGAAATTCAATCCTAACTGAGACCTTACACCGTTT |
| | | CTCATTAGAAGGAGATGCTCCTGTCTCCTGGACAGAAACAAAAAAACAA |
| | | tCTTTTAAACAGACTGGAGAGTTTGGGGAAAAAAGGAAGAATTCTATTC |
| | | TCAATCCAATCAACTCTACGCTTCAGGCACGAAGGAGGCAGTCTGTCCT |
| | | GAACCTGATGACACACTCAGTTAACCAAGGTCAGAACATTCACCGAAAG |
| | | ACAACAGCATCCACACGAAAAGTGTCACTGGCCCCTCAGGCAAACTTG |
| | | ACTGAACTGGATATATATTCAAGAAGGTTATCTCAAGAAACTGGCTTGG |
| | | AAATAAGTGAAGAAATTAACGAAGAAGACTTAAAGGAGTGCCTTTTTGA |
| | | TGATATGGAGAGCATACCAGCAGTGACTACATGGAACACATACCTTCGA |
| | | TATATTACTGTCCACAAGAGCTTAATTTTTGTGCTAATTTGGTGCTTAGT |
| | | AATTTTTCTGGCAGAGGTGGCTGCTTCTTTGGTTGTGCTGTGGCTCCTT |
| | | GGAAACACTCCTCTTCAAGACAAAGGGAATAGTACTCATAGTAGAAATA |
| | | ACAGCTATGCAGTGATTATCACCAGCACCAGTTCGTATTATGTGTTTTAC |
| | | ATTTACGTGGGAGTAGCCGACACTTTGCTTGCTATGGGATTCTTCAGAG |
| | | GTCTACCACTGGTGCATACTCTAATCACAGTGTCGAAAATTTTACACCA |
| | | CAAAATGTTACATTCTGTTCTTCAAGCACCTATGTCAACCCTCAACACGT |
| | | TGAAAGCAGGTGGGATTCTTAATAGATTCTCCAAAGATATAGCAATTTTG |
| | | GATGACCTTCTGCCTCTTACCATATTTGACTTCATCCAGTTGTTATTAAT |
| | | TGTGATTGGAGCTATAGCAGTTGTCGCAGTTTTACAACCCTACATCTTT |
| | | GTTGCAACAGTGCCAGTGATAGTGGCTTTTATTATGTTGAGAGCATATT |
| | | TCCTCCAAACCTCACAGCAACTCAAACAACTGGAATCTGAAGGCAGGA |
| | | GTCCAATTTTCACTCATCTTGTTACAAGCTTAAAAGGACTATGGACACTT |
| | | CGTGCCTTCGGACGGCAGCCTTACTTTGAAACTCTGTTCCACAAAGCTC |
| | | TGAATTTACATACTGCCAACTGGTTCTTGTACCTGTCAACACTGCGCTG |
| | | GTTCCAAATGAGAATAGAAATGATTTTTGTCATCTTCTTCATTGCTGTTA |
| | | CCTTCATTTCCATTTTAACAACAGGAGAAGGAGAAGGAAGAGTTGGTAT |
| | | TATCCTGACTTTAGCCATGAATATCATGAGTACATTGCAGTGGGCTGTA |
| | | AACTCCAGCATAGATGTGGATAGCTTGATGCGATCTGTGAGCCGAGTC |
| | | TTTAAGTTCATTGACATGCCAACAGAAGGTAAACCTACCAAGTCAACCA |
| | | AACCATACAAGAATGGCCAACTCTCGAAAGTTATGATTATTGAGAATTCA |
| | | CACGTGAAGAAAGATGACATCTGGCCCTCAGGGGGCCAAATGACTGTC |
| | | AAAGATCTCACAGCAAAATACACAGAAGGTGGAAATGCCATATTAGAGA |
| | | ACATTTCCTTCTCAATAAGTCCTGGCCAGAGGGTGGGCCTCTTGGGAA |
| | | GAACTGGATCAGGGAAGAGTACTTTGTTATCAGCTTTTTTGAGACTACT |
| | | GAACACTGAAGGAGAAATCCAGATCGATGGTGTGTCTTGGGATTCAATA |
| | | ACTTTGCAACAGTGGAGGAAAGCCTTTGGAGTGATACCACAGAAAGTAT |
| | | TTATTTTTTCTGGAACATTTAGAAAAAACTTGGATCCCTATGAACAGTGG |
| | | AGTGATCAAGAAATATGGAAAGTTGCAGATGAGGTTGGGCTCAGATCT |
| | | GTGATAGAACAGTTTCCTGGGAAGCTTGACTTTGTCCTTGTGGATGGG |
| | | GGCTGTGTCCTAAGCCATGGCCACAAGCAGTTGATGTGCTTGGCTAGA |
| | | TCTGTTCTCAGTAAGGCGAAGATCTTGCTGCTTGATGAACCCAGTGCTC |
| | | ATTTGGATCCAGTAACATACCAAATAATTAGAAGAACTCTAAAACAAGCA |
| | | TTTGCTGATTGCACAGTAATTCTCTGTGAACACAGGATAGAAGCAATGC |
| | | TGGAATGCCAACAATTTTTGGTCATAGAAGAGAACAAAGTGCGGCAGTA |
| | | CGATTCCATCCAGAAACTGCTGAACGAGAGGAGCCTCTTCCGGCAAGC |
| | | CATCAGCCCCTCCGACAGGGTGAAGCTCTTTCCCCACCGGAACTCAAG |
| | | CAAGTGCAAGTCTAAGCCCCAGATTGCTGCTCTGAAAGAGGAGACAGA |
| | | AGAAGAGGTGCAAGATACAAGGCTTTAG |
| 8 | F5 | GAATTCGTGGTGAGCGTCTGGGCATGTCTGGGCATGTCTGGGCATGTC |
| | Enhancer, | TGGGCATGTCGGGCATTCTGGGCGTCTGGGCATGTCTGGGCATGTCTG |
| | Tg83 | GGCATCTCGAGAACGGTGACGTGCACGCGTGGGCGGAGCCATCACGC |
| | Promoter, | AGGTTGCTATATAAGCAGAGCTCGTTTAGTGAACCGTCAGAGTCGAGC |
| | 5′-UTR, | CCGAGAGACCATGCAGAGGTCGCCTCTGGAAAAGGCCAGCGTTGTCTC |
| | hCFTRΔR, | CAAACTTTTTTTCAGCTGGACCAGACCAATTTTGAGGAAAGGATACAGA |
| | 3′-UTR | CAGCGCCTGGAATTGTCAGACATATACCAAATCCCTTCTGTTGATTCTG |
| | | CTGACAATCTATCTGAAAAATTGGAAAGAGAATGGGATAGAGAGCTGGC |
| | | TTCAAAGAAAAATCCTAAACTCATTAATGCCCTTCGGCGATGTTTTTTCT |
| | | GGAGATTTATGTTCTATGGAATCTTTTTATATTTAGGGGAAGTCACCAAA |
| | | GCAGTACAGCCTCTCTTACTGGGAAGAATCATAGCTTCCTATGACCCGG |
| | | ATAACAAGGAGGAACGCTCTATCGCGATTTATCTAGGCATAGGCTTATG |
| | | CCTTCTCTTTATTGTGAGGACACTGCTCCTACACCCAGCCATTTTTGGC |
| | | CTTCATCACATTGGAATGCAGATGAGAATAGCTATGTTTAGTTTGATTTA |
| | | TAAGAAGACTTTAAAGCTGTCAAGCCGTGTTCTAGATAAAATAAGTATTG |
| | | GACAACTTGTTAGTCTCCTTTCCAACAACCTGAACAAATTTGATGAAGG |
| | | ACTTGCATTGGCACATTTCGTGTGGATCGCTCCTTTGCAAGTGGCACTC |
| | | CTCATGGGGCTAATCTGGGAGTTGTTACAGGCGTCTGCCTTCTGTGGA |
| | | CTTGGTTTCCTGATAGTCCTTGCCCTTTTTCAGGCTGGGCTAGGGAGAA |
| | | TGATGATGAAGTACAGAGATCAGAGAGCTGGGAAGATCAGTGAAAGAC |
| | | TTGTGATTACCTCAGAAATGATCGAGAACATCCAATCTGTTAAGGCATA |
| | | CTGCTGGGAAGAAGCAATGGAAAAAATGATTGAAAACTTAAGACAAACA |
| | | GAACTGAAACTGACTCGGAAGGCAGCCTATGTGAGATACTTCAATAGCT |
| | | CAGCCTTCTTCTTCTCAGGGTTCTTTGTGGTGTTTTTATCTGTGCTTCCC |
| | | TATGCACTAATCAAAGGAATCATCCTCCGGAAAATATTCACCACCATCT |
| | | CATTCTGCATTGTTCTGCGCATGGCGGTCACTCGGCAATTTCCCTGGG |
| | | CTGTACAAACATGGTATGACTCTCTTGGAGCAATAAACAAAATACAGGA |
| | | TTTCTTACAAAAGCAAGAATATAAGACATTGGAATATAACTTAACGACTA |
| | | CAGAAGTAGTGATGGAGAATGTAACAGCCTTCTGGGAGGAGGGATTTG |
| | | GGGAATTATTTGAGAAAGCAAAACAAAACAATAACAATAGAAAAACTTCT |
| | | AATGGTGATGACAGCCTCTTCTTCAGTAATTTCTCACTTCTTGGTACTCC |
| | | TGTCCTGAAAGATATTAATTTCAAGATAGAAAGAGGACAGTTGTTGGCG |
| | | GTTGCTGGATCCACTGGAGCAGGCAAGACTTCACTTCTAATGATGATTA |
| | | TGGGAGAACTGGAGCCTTCAGAGGGTAAAATTAAGCACAGTGGAAGAA |
| | | TTTCATTCTGTTCTCAGTTTTCCTGGATTATGCCTGGCACCATTAAAGAA |
| | | AATATCATCTTTGGTGTTTCCTATGATGAATATAGATACAGAAGCGTCAT |
| | | CAAAGCATGCCAACTAGAAGAGGACATCTCCAAGTTTGCAGAGAAAGA |
| | | CAATATAGTTCTTGGAGAAGGTGGAATCACACTGAGTGGAGGTCAACG |
| | | AGCAAGAATTTCTTTAGCAAGAGCAGTATACAAAGATGCTGATTTGTATT |
| | | TATTAGACTCTCCTTTTGGATACCTAGATGTTTTAACAGAAAAAGAAATA |
| | | TTTGAAAGCTGTGTCTGTAAACTGATGGCTAACAAAACTAGGATTTTGG |
| | | TCACTTCTAAAATGGAACATTTAAAGAAAGCTGACAAAATATTAATTTTG |
| | | CATGAAGGTAGCAGCTATTTTTATGGGACATTTTCAGAACTCCAAAATCT |
| | | ACAGCCAGACTTTAGCTCAAAACTCATGGGATGTGATTCTTTCGACCAA |
| | | TTTAGTGCAGAAAGAAGAAATTCAATCCTAACTGAGACCTTACACCGTTT |
| | | CTCATTAGAAGGAGATGCTCCTGTCTCCTGGACAGAAACAAAAAAACAA |
| | | TCTTTTAAACAGACTGGAGAGTTTGGGGAAAAAAGGAAGAATTCTATTC |
| | | TCAATCCAATCAACTCTACGCTTCAGGCACGAAGGAGGCAGTCTGTCCT |
| | | GAACCTGATGACACACTCAGTTAACCAAGGTCAGAACATTCACCGAAAG |
| | | ACAACAGCATCCACACGAAAAGTGTCACTGGCCCCTCAGGCAAACTTG |
| | | ACTGAACTGGATATATATTCAAGAAGGTTATCTCAAGAAACTGGCTTGG |
| | | AAATAAGTGAAGAAATTAACGAAGAAGACTTAAAGGAGTGCCTTTTTGA |
| | | TGATATGGAGAGCATACCAGCAGTGACTACATGGAACACATACCTTCGA |
| | | TATATTACTGTCCACAAGAGCTTAATTTTTGTGCTAATTTGGTGCTTAGT |
| | | AATTTTTCTGGCAGAGGTGGCTGCTTCTTTGGTTGTGCTGTGGCTCCTT |
| | | GGAAACACTCCTCTTCAAGACAAAGGGAATAGTACTCATAGTAGAAATA |
| | | ACAGCTATGCAGTGATTATCACCAGCACCAGTTCGTATTATGTGTTTTAC |
| | | ATTTACGTGGGAGTAGCCGACACTTTGCTTGCTATGGGATTCTTCAGAG |
| | | GTCTACCACTGGTGCATACTCTAATCACAGTGTCGAAAATTTTACACCA |
| | | CAAAATGTTACATTCTGTTCTTCAAGCACCTATGTCAACCCTCAACACGT |
| | | TGAAAGCAGGTGGGATTCTTAATAGATTCTCCAAAGATATAGCAATTTTG |
| | | GATGACCTTCTGCCTCTTACCATATTTGACTTCATCCAGTTGTTATTAAT |
| | | TGTGATTGGAGCTATAGCAGTTGTCGCAGTTTTACAACCCTACATCTTT |
| | | GTTGCAACAGTGCCAGTGATAGTGGCTTTTATTATGTTGAGAGCATATT |
| | | TCCTCCAAACCTCACAGCAACTCAAACAACTGGAATCTGAAGGCAGGA |
| | | GTCCAATTTCACTCATCTTGTTACAAGCTTAAAAGGACTATGGACACTT |
| | | CGTGCCTTCGGACGGCAGCCTTACTTTGAAACTCTGTTCCACAAAGCTC |
| | | TGAATTTACATACTGCCAACTGGTTCTTGTACCTGTCAACACTGCGCTG |
| | | GTTCCAAATGAGAATAGAAATGATTTTTGTCATCTTCTTCATTGCTGTTA |
| | | CCTTCATTTCCATTTAACAACAGGAGAAGGAGAAGGAAGAGTTGGTAT |
| | | TATCCTGACTTTAGCCATGAATATCATGAGTACATTGCAGTGGGCTGTA |
| | | AACTCCAGCATAGATGTGGATAGCTTGATGCGATCTGTGAGCCGAGTC |
| | | TTTAAGTTCATTGACATGCCAACAGAAGGTAAACCTACCAAGTCAACCA |
| | | AACCATACAAGAATGGCCAACTCTCGAAAGTTATGATTATTGAGAATTCA |
| | | CACGTGAAGAAAGATGACATCTGGCCCTCAGGGGGCCAAATGACTGTC |
| | | AAAGATCTCACAGCAAAATACACAGAAGGTGGAAATGCCATATTAGAGA |
| | | ACATTTCCTTCTCAATAAGTCCTGGCCAGAGGGTGGGCCTCTTGGGAA |
| | | GAACTGGATCAGGGAAGAGTACTTTGTTATCAGCTTTTTTGAGACTACT |
| | | GAACACTGAAGGAGAAATCCAGATCGATGGTGTGTCTTGGGATTCAATA |
| | | ACTTTGCAACAGTGGAGGAAAGCCTTTGGAGTGATACCACAGAAAGTAT |
| | | TTATTTTTTCTGGAACATTTAGAAAAAACTTGGATCCCTATGAACAGTGG |
| | | AGTGATCAAGAAATATGGAAAGTTGCAGATGAGGTTGGGCTCAGATCT |
| | | GTGATAGAACAGTTTCCTGGGAAGCTTGACTTTGTCCTTGTGGATGGG |
| | | GGCTGTGTCCTAAGCCATGGCCACAAGCAGTTGATGTGCTTGGCTAGA |
| | | TCTGTTCTCAGTAAGGCGAAGATCTTGCTGCTTGATGAACCCAGTGCTC |
| | | ATTTGGATCCAGTAACATACCAAATAATTAGAAGAACTCTAAAACAAGCA |
| | | TTTGCTGATTGCACAGTAATTCTCTGTGAACACAGGATAGAAGCAATGC |
| | | TGGAATGCCAACAATTTTTGGTCATAGAAGAGAACAAAGTGCGGCAGTA |
| | | CGATTCCATCCAGAAACTGCTGAACGAGAGGAGCCTCTTCCGGCAAGC |
| | | CATCAGCCCCTCCGACAGGGTGAAGCTCTTTCCCCACCGGAACTCAAG |
| | | CAAGTGCAAGTCTAAGCCCCAGATTGCTGCTCTGAAAGAGGAGACAGA |
| | | AGAAGAGGTGCAAGATACAAGGCTTTAGAGAGCAGCATAAATGTTGAC |
| | | ATGGGACATTTGCTCATGGAATTGG |
| 9 | 5′ AAV ITR | TTGGCCACTCCCTCTCTGCGCGCTCGCTCGCTCACTGAGGCCGGGCG |
| | | ACCAAAGGTCGCOCGACGCCCGGGCTTTGCCCGGGCGGCCTCAGTGA |
| | | GCGAGCGAGCGCGCAGAGAGGGAGTGGCCAACTCCATCACTAGGGGT |
| | | TCCT |
| 10 | 3′ AAV ITR | AGGAACCCCTAGTGATGGAGTTGGCCACTCCCTCTCTGCGCGCTCGCT |
| | | CGCTCACTGAGGCCGCCCGGGCAAAGCCCGGGCGTCGGGCGACCTTT |
| | | GGTCGCCCGGCCTCAGTGAGCGAGCGAGCGCGCAGAGAGGGAGTGG |
| | | CCAA |
| 11 | 5′ AAV ITR | TTGGCCACTCCCTCTCTGCGCGCTCGCTCGCTCACTGAGGCCGGGCG |
| | through 3′ | ACCAAAGGTCGCCCGACGCCCGGGCTTTGCCCGGGCGGCCTCAGTGA |
| | ITR | GCGAGCGAGCGCGCAGAGAGGGAGTGGCCAACTCCATCACTAGGGGT |
| | | TCCTCAGATCTGAATTCGTGGTGAGCGTCTGGGCATGTCTGGGCATGT |
| | | CTGGGCATGTCTGGGCATGTCGGGCATTCTGGGCGTCTGGGCATGTCT |
| | | GGGCATGTCTGGGCATCTCGAGAACGGTGACGTGCACGCGTGGGCGG |
| | | AGCCATCACGCAGGTTGCTATATAAGCAGAGCTCGTTTAGTGAACCGTC |
| | | AGAGTCGAGCCCGAGAGACCATGCAGAGGTCGCCTCTGGAAAAGGCC |
| | | AGCGTTGTCTCCAAACTTTTTTTCAGCTGGACCAGACCAATTTTGAGGA |
| | | AAGGATACAGACAGCGCCTGGAATTGTCAGACATATACCAAATCCCTTC |
| | | TGTTGATTCTGCTGACAATCTATCTGAAAAATTGGAAAGAGAATGGGAT |
| | | AGAGAGCTGGCTTCAAAGAAAAATCCTAAACTCATTAATGCCCTTCGGC |
| | | GATGTTTTTTCTGGAGATTTATGTTCTATGGAATCTTTTTATATTTAGGG |
| | | GAAGTCACCAAAGCAGTACAGCCTCTCTTACTGGGAAGAATCATAGCTT |
| | | CCTATGACCCGGATAACAAGGAGGAACGCTCTATCGCGATTTATCTAG |
| | | GCATAGGCTTATGCCTTCTCTTTATTGTGAGGACACTGCTCCTACACCC |
| | | AGCCATTTTTGGCCTTCATCACATTGGAATGCAGATGAGAATAGCTATG |
| | | TTTAGTTTGATTTATAAGAAGACTTTAAAGCTGTCAAGCCGTGTTCTAGA |
| | | TAAAATAAGTATTGGACAACTTGTTAGTCTCCTTTCCAACAACCTGAACA |
| | | AATTTGATGAAGGACTTGCATTGGCACATTTCGTGTGGATCGCTCCTTT |
| | | GCAAGTGGCACTCCTCATGGGGCTAATCTGGGAGTTGTTACAGGCGTC |
| | | TGCCTTCTGTGGACTTGGTTTCCTGATAGTCCTTGCCCTTTTTCAGGCT |
| | | GGGCTAGGGAGAATGATGATGAAGTACAGAGATCAGAGAGCTGGGAA |
| | | GATCAGTGAAAGACTTGTGATTACCTCAGAAATGATCGAGAACATCCAA |
| | | TCTGTTAAGGCATACTGCTGGGAAGAAGCAATGGAAAAAATGATTGAAA |
| | | ACTTAAGACAAACAGAACTGAAACTGACTCGGAAGGCAGCCTATGTGA |
| | | GATACTTCAATAGCTCAGCCTTCTTCTTCTCAGGGTTCTTTGTGGTGTTT |
| | | TTATCTGTGCTTCCCTATGCACTAATCAAAGGAATCATCCTCCGGAAAAT |
| | | ATTCACCACCATCTCATTCTGCATTGTTCTGCGCATGGCGGTCACTCGG |
| | | CAATTTCCCTGGGCTGTACAAACATGGTATGACTCTCTTGGAGCAATAA |
| | | ACAAAATACAGGATTTCTTACAAAAGCAAGAATATAAGACATTGGAATAT |
| | | AACTTAACGACTACAGAAGTAGTGATGGAGAATGTAACAGCCTTCTGGG |
| | | AGGAGGGATTTGGGGAATTATTTGAGAAAGCAAAACAAAACAATAACAA |
| | | TAGAAAAACTTCTAATGGTGATGACAGCCTCTTCTTCAGTAATTTCTCAC |
| | | TTCTTGGTACTCCTGTCCTGAAAGATATTAATTTCAAGATAGAAAGAGGA |
| | | CAGTTGTTGGCGGTTGCTGGATCCACTGGAGCAGGCAAGACTTCACTT |
| | | CTAATGATGATTATGGGAGAACTGGAGCCTTCAGAGGGTAAAATTAAGC |
| | | ACAGTGGAAGAATTTCATTCTGTTCTCAGTTTTCCTGGATTATGCCTGG |
| | | CACCATTAAAGAAAATATCATCTTTGGTGTTTCCTATGATGAATATAGAT |
| | | ACAGAAGCGTCATCAAAGCATGCCAACTAGAAGAGGACATCTCCAAGTT |
| | | TGCAGAGAAAGACAATATAGTTCTTGGAGAAGGTGGAATCACACTGAGT |
| | | GGAGGTCAACGAGCAAGAATTTCTTTAGCAAGAGCAGTATACAAAGATG |
| | | CTGATTTGTATTTATTAGACTCTCCTTTTGGATACCTAGATGTTTTAACA |
| | | GAAAAAGAAATATTTGAAAGCTGTGTCTGTAAACTGATGGCTAACAAAA |
| | | CTAGGATTTTGGTCACTTCTAAAATGGAACATTTAAAGAAAGCTGACAAA |
| | | ATATTAATTTTGCATGAAGGTAGCAGCTATTTTTATGGGACATTTTCAGA |
| | | ACTCCAAAATCTACAGCCAGACTTTAGCTCAAAACTCATGGGATGTGAT |
| | | TCTTTCGACCAATTTAGTGCAGAAAGAAGAAATTCAATCCTAACTGAGA |
| | | CCTTACACCGTTTCTCATTAGAAGGAGATGCTCCTGTCTCCTGGACAGA |
| | | AACAAAAAAACAATCTTTTAAACAGACTGGAGAGTTTGGGGAAAAAAGG |
| | | AAGAATTCTATTCTCAATCCAATCAACTCTACGCTTCAGGCACGAAGGA |
| | | GGCAGTCTGTCCTGAACCTGATGACACACTCAGTTAACCAAGGTCAGA |
| | | ACATTCACCGAAAGACAACAGCATCCACACGAAAAGTGTCACTGGCCC |
| | | CTCAGGCAAACTTGACTGAACTGGATATATATTCAAGAAGGTTATCTCA |
| | | AGAAACTGGCTTGGAAATAAGTGAAGAAATTAACGAAGAAGACTTAAAG |
| | | GAGTGCCTTTTTGATGATATGGAGAGCATACCAGCAGTGACTACATGGA |
| | | ACACATACCTTCGATATATTACTGTCCACAAGAGCTTAATTTTTGTGCTA |
| | | ATTTGGTGCTTAGTAATTTTTCTGGCAGAGGTGGCTGCTTCTTTGGTTG |
| | | TGCTGTGGCTCCTTGGAAACACTCCTCTTCAAGACAAAGGGAATAGTAC |
| | | TCATAGTAGAAATAACAGCTATGCAGTGATTATCACCAGCACCAGTTCG |
| | | TATTATGTGTTTTACATTTACGTGGGAGTAGCCGACACTTTGCTTGCTAT |
| | | GGGATTCTTCAGAGGTCTACCACTGGTGCATACTCTAATCACAGTGTCG |
| | | AAAATTTTACACCACAAAATGTTACATTCTGTTCTTCAAGCACCTATGTC |
| | | AACCCTCAACACGTTGAAAGCAGGTGGGATTCTTAATAGATTCTCCAAA |
| | | GATATAGCAATTTTGGATGACCTTCTGCCTCTTACCATATTTGACTTCAT |
| | | CCAGTTGTTATTAATTGTGATTGGAGCTATAGCAGTTGTCGCAGTTTTAC |
| | | AACCCTACATCTTTGTTGCAACAGTGCCAGTGATAGTGGCTTTTATTATG |
| | | TTGAGAGCATATTTCCTCCAAACCTCACAGCAACTCAAACAACTGGAAT |
| | | CTGAAGGCAGGAGTCCAATTTTCACTCATCTTGTTACAAGCTTAAAAGG |
| | | ACTATGGACACTTCGTGCCTTCGGACGGCAGCCTTACTTTGAAACTCTG |
| | | TTCCACAAAGCTCTGAATTTACATACTGCCAACTGGTTCTTGTACCTGTC |
| | | AACACTGCGCTGGTTCCAAATGAGAATAGAAATGATTTTTGTCATCTTCT |
| | | TCATTGCTGTTACCTTCATTTCCATTTTAACAACAGGAGAAGGAGAAGG |
| | | AAGAGTTGGTATTATCCTGACTTTAGCCATGAATATCATGAGTACATTGC |
| | | AGTGGGCTGTAAACTCCAGCATAGATGTGGATAGCTTGATGCGATCTGT |
| | | GAGCCGAGTCTTTAAGTTCATTGACATGCCAACAGAAGGTAAACCTACC |
| | | AAGTCAACCAAACCATACAAGAATGGCCAACTCTCGAAAGTTATGATTA |
| | | TTGAGAATTCACACGTGAAGAAAGATGACATCTGGCCCTCAGGGGGCC |
| | | AAATGACTGTCAAAGATCTCACAGCAAAATACACAGAAGGTGGAAATGC |
| | | CATATTAGAGAACATTTCCTTCTCAATAAGTCCTGGCCAGAGGGTGGGC |
| | | CTCTTGGGAAGAACTGGATCAGGGAAGAGTACTTTGTTATCAGCTTTTT |
| | | TGAGACTACTGAACACTGAAGGAGAAATCCAGATCGATGGTGTGTCTTG |
| | | GGATTCAATAACTTTGCAACAGTGGAGGAAAGCCTTTGGAGTGATACCA |
| | | CAGAAAGTATTTATTTTTTCTGGAACATTTAGAAAAAACTTGGATCCCTA |
| | | TGAACAGTGGAGTGATCAAGAAATATGGAAAGTTGCAGATGAGGTTGG |
| | | GCTCAGATCTGTGATAGAACAGTTTCCTGGGAAGCTTGACTTTGTCCTT |
| | | GTGGATGGGGGCTGTGTCCTAAGCCATGGCCACAAGCAGTTGATGTGC |
| | | TTGGCTAGATCTGTTCTCAGTAAGGCGAAGATCTTGCTGCTTGATGAAC |
| | | CCAGTGCTCATTTGGATCCAGTAACATACCAAATAATTAGAAGAACTCTA |
| | | AAACAAGCATTTGCTGATTGCACAGTAATTCTCTGTGAACACAGGATAG |
| | | AAGCAATGCTGGAATGCCAACAATTTTTGGTCATAGAAGAGAACAAAGT |
| | | GCGGCAGTACGATTCCATCCAGAAACTGCTGAACGAGAGGAGCCTCTT |
| | | CCGGCAAGCCATCAGCCCCTCCGACAGGGTGAAGCTCTTTCCCCACCG |
| | | GAACTCAAGCAAGTGCAAGTCTAAGCCCCAGATTGCTGCTCT GAAAGA |
| | | GGAGACAGAAGAAGAGGTGCAAGATACAAGGCTTTAGAGAGCAGCATA |
| | | AATGTTGACATGGGACATTTGCTCATGGAATTGGCAGGCCTAATAAAGA |
| | | GCTCAGATGCATCGATCAGAGTGTGTTGGTTTTTTGTGTGTACTGAGGA |
| | | ACCCCTAGTGATGGAGTTGGCCACTCCCTCTCTGCGCGCTCGCTCGCT |
| | | CACTGAGGCCGCCCGGGCAAAGCCCGGGCGTCGGGCGACCTTTGGTC |
| | | GCCCGGCCTCAGTGAGCGAGCGAGCGCGCAGAGAGGGAGTGGCCAA |
| 12 | pAV- | TTGGCCACTCCCTCTCTGCGCGCTCGCTCGCTCACTGAGGCCGGGCG |
| | F5tg83- | ACCAAAGGTCGCCCGACGCCCGGGCTTTGCCCGGGCGGCCTCAGTGA |
| | hCFTR-dR | GCGAGCGAGCGCGCAGAGAGGGAGTGGCCAACTCCATCACTAGGGGT |
| | vector | TCCTCAGATCTGAATTCGTGGTGAGCGTCTGGGCATGTCTGGGCATGT |
| | | CTGGGCATGTCTGGGCATGTCGGGCATTCTGGGCGTCTGGGCATGTCT |
| | | GGGCATGTCTGGGCATCTCGAGAACGGTGACGTGCACGCGTGGGCGG |
| | | AGCCATCACGCAGGTTGCTATATAAGCAGAGCTCGTTTAGTGAACCGTC |
| | | AGAGTCGAGCCCGAGAGACCATGCAGAGGTCGCCTCTGGAAAAGGCC |
| | | AGCGTTGTCTCCAAACTTTTTTTCAGCTGGACCAGACCAATTTTGAGGA |
| | | AAGGATACAGACAGCGCCTGGAATTGTCAGACATATACCAAATCCCTTC |
| | | TGTTGATTCTGCTGACAATCTATCTGAAAAATTGGAAAGAGAATGGGAT |
| | | AGAGAGCTGGCTTCAAAGAAAAATCCTAAACTCATTAATGCCCTTCGGC |
| | | GATGTTTTTTCTGGAGATTTATGTTCTATGGAATCTTTTTATATTTAGGG |
| | | GAAGTCACCAAAGCAGTACAGCCTCTCTTACTGGGAAGAATCATAGCTT |
| | | CCTATGACCCGGATAACAAGGAGGAACGCTCTATCGCGATTTATCTAG |
| | | GCATAGGCTTATGCCTTCTCTTTATTGTGAGGACACTGCTCCTACACCC |
| | | AGCCATTTTTGGCCTTCATCACATTGGAATGCAGATGAGAATAGCTATG |
| | | TTTAGTTTGATTTATAAGAAGACTTTAAAGCTGTCAAGCCGTGTTCTAGA |
| | | TAAAATAAGTATTGGACAACTTGTTAGTCTCCTTTCCAACAACCTGAACA |
| | | AATTTGATGAAGGACTTGCATTGGCACATTTCGTGTGGATCGCTCCTTT |
| | | GCAAGTGGCACTCCTCATGGGGCTAATCTGGGAGTTGTTACAGGCGTC |
| | | TGCCTTCTGTGGACTTGGTTTCCTGATAGTCCTTGCCCTTTTTCAGGCT |
| | | GGGCTAGGGAGAATGATGATGAAGTACAGAGATCAGAGAGCTGGGAA |
| | | GATCAGTGAAAGACTTGTGATTACCTCAGAAATGATCGAGAACATCCAA |
| | | TCTGTTAAGGCATACTGCTGGGAAGAAGCAATGGAAAAAATGATTGAAA |
| | | ACTTAAGACAAACAGAACTGAAACTGACTCGGAAGGCAGCCTATGTGA |
| | | GATACTTCAATAGCTCAGCCTTCTTCTTCTCAGGGTTCTTTGTGGTGTTT |
| | | TTATCTGTGCTTCCCTATGCACTAATCAAAGGAATCATCCTCCGGAAAAT |
| | | ATTCACCACCATCTCATTCTGCATTGTTCTGCGCATGGCGGTCACTCGG |
| | | CAATTTCCCTGGGCTGTACAAACATGGTATGACTCTCTTGGAGCAATAA |
| | | ACAAAATACAGGATTTCTTACAAAAGCAAGAATATAAGACATTGGAATAT |
| | | AACTTAACGACTACAGAAGTAGTGATGGAGAATGTAACAGCCTTCTGGG |
| | | AGGAGGGATTTGGGGAATTATTTGAGAAAGCAAAACAAAACAATAACAA |
| | | TAGAAAAACTTCTAATGGTGATGACAGCCTCTTCTTCAGTAATTTCTCAC |
| | | TTCTTGGTACTCCTGTCCTGAAAGATATTAATTTCAAGATAGAAAGAGGA |
| | | CAGTTGTTGGCGGTTGCTGGATCCACTGGAGCAGGCAAGACTTCACTT |
| | | CTAATGATGATTATGGGAGAACTGGAGCCTTCAGAGGGTAAAATTAAGC |
| | | ACAGTGGAAGAATTTCATTCTGTTCTCAGTTTTCCTGGATTATGCCTGG |
| | | CACCATTAAAGAAAATATCATCTTTGGTGTTTCCTATGATGAATATAGAT |
| | | ACAGAAGCGTCATCAAAGCATGCCAACTAGAAGAGGACATCTCCAAGTT |
| | | TGCAGAGAAAGACAATATAGTTCTTGGAGAAGGTGGAATCACACTGAGT |
| | | GGAGGTCAACGAGCAAGAATTTCTTTAGCAAGAGCAGTATACAAAGATG |
| | | CTGATTTGTATTTATTAGACTCTCCTTTTGGATACCTAGATGTTTTAACA |
| | | GAAAAAGAAATATTTGAAAGCTGTGTCTGTAAACTGATGGCTAACAAAA |
| | | CTAGGATTGGTCACTTCTAAAATGGAACATTTAAAGAAAGCTGACAAA |
| | | ATATTAATTTTGCATGAAGGTAGCAGCTATTTTTATGGGACATTTTCAGA |
| | | ACTCCAAAATCTACAGCCAGACTTTAGCTCAAAACTCATGGGATGTGAT |
| | | TCTTTCGACCAATTTAGTGCAGAAAGAAGAAATTCAATCCTAACTGAGA |
| | | CCTTACACCGTTTCTCATTAGAAGGAGATGCTCCTGTCTCCTGGACAGA |
| | | AACAAAAAAACAATCTTTTAAACAGACTGGAGAGTTTGGGGAAAAAAGG |
| | | AAGAATTCTATTCTCAATCCAATCAACTCTACGCTTCAGGCACGAAGGA |
| | | GGCAGTCTGTCCTGAACCTGATGACACACTCAGTTAACCAAGGTCAGA |
| | | ACATTCACCGAAAGACAACAGCATCCACACGAAAAGTGTCACTGGCCC |
| | | CTCAGGCAAACTTGACTGAACTGGATATATATTCAAGAAGGTTATCTCA |
| | | AGAAACTGGCTTGGAAATAAGTGAAGAAATTAACGAAGAAGACTTAAAG |
| | | GAGTGCCTTTTTGATGATATGGAGAGCATACCAGCAGTGACTACATGGA |
| | | ACACATACCTTCGATATATTACTGTCCACAAGAGCTTAATTTTTGTGCTA |
| | | ATTTGGTGCTTAGTAATTTTTCTGGCAGAGGTGGCTGCTTCTTTGGTTG |
| | | TGCTGTGGCTCCTTGGAAACACTCCTCTTCAAGACAAAGGGAATAGTAC |
| | | TCATAGTAGAAATAACAGCTATGCAGTGATTATCACCAGCACCAGTTCG |
| | | TATTATGTGTTTTACATTTACGTGGGAGTAGCCGACACTTTGCTTGCTAT |
| | | GGGATTCTTCAGAGGTCTACCACTGGTGCATACTCTAATCACAGTGTCG |
| | | AAAATTTTACACCACAAAATGTTACATTCTGTTCTTCAAGCACCTATGTC |
| | | AACCCTCAACACGTTGAAAGCAGGTGGGATTCTTAATAGATTCTCCAAA |
| | | GATATAGCAATTTTGGATGACCTTCTGCCTCTTACCATATTTGACTTCAT |
| | | CCAGTTGTTATTAATTGTGATTGGAGCTATAGCAGTTGTCGCAGTTTTAC |
| | | AACCCTACATCTTTGTTGCAACAGTGCCAGTGATAGTGGCTTTTATTATG |
| | | TTGAGAGCATATTTCCTCCAAACCTCACAGCAACTCAAACAACTGGAAT |
| | | CTGAAGGCAGGAGTCCAATTTTCACTCATCTTGTTACAAGCTTAAAAGG |
| | | ACTATGGACACTTCGTGCCTTCGGACGGCAGCCTTACTTTGAAACTCTG |
| | | TTCCACAAAGCTCTGAATTTACATACTGCCAACTGGTTCTTGTACCTGTC |
| | | AACACTGCGCTGGTTCCAAATGAGAATAGAAATGATTTTTGTCATCTTCT |
| | | TCATTGCTGTTACCTTCATTTCCATTTTAACAACAGGAGAAGGAGAAGG |
| | | AAGAGTTGGTATTATCCTGACTTTAGCCATGAATATCATGAGTACATTGC |
| | | AGTGGGCTGTAAACTCCAGCATAGATGTGGATAGCTTGATGCGATCTGT |
| | | GAGCCGAGTCTTTAAGTTCATTGACATGCCAACAGAAGGTAAACCTACC |
| | | AAGTCAACCAAACCATACAAGAATGGCCAACTCTCGAAAGTTATGATTA |
| | | TTGAGAATTCACACGTGAAGAAAGATGACATCTGGCCCTCAGGGGGCC |
| | | AAATGACTGTCAAAGATCTCACAGCAAAATACACAGAAGGTGGAAATGC |
| | | CATATTAGAGAACATTTCCTTCTCAATAAGTCCTGGCCAGAGGGTGGGC |
| | | CTCTTGGGAAGAACTGGATCAGGGAAGAGTACTTTGTTATCAGCTTTTT |
| | | TGAGACTACTGAACACTGAAGGAGAAATCCAGATCGATGGTGTGTCTTG |
| | | GGATTCAATAACTTTGCAACAGTGGAGGAAAGCCTTTGGAGTGATACCA |
| | | CAGAAAGTATTTATTTTTTCTGGAACATTTAGAAAAAACTTGGATCCCTA |
| | | TGAACAGTGGAGTGATCAAGAAATATGGAAAGTTGCAGATGAGGTTGG |
| | | GCTCAGATCTGTGATAGAACAGTTTCCTGGGAAGCTTGACTTTGTCCTT |
| | | GTGGATGGGGGCTGTGTCCTAAGCCATGGCCACAAGCAGTTGATGTGC |
| | | TTGGCTAGATCTGTTCTCAGTAAGGCGAAGATCTTGCTGCTTGATGAAC |
| | | CCAGTGCTCATTTGGATCCAGTAACATACCAAATAATTAGAAGAACTCTA |
| | | AAACAAGCATTTGCTGATTGCACAGTAATTCTCTGTGAACACAGGATAG |
| | | AAGCAATGCTGGAATGCCAACAATTTTTGGTCATAGAAGAGAACAAAGT |
| | | GCGGCAGTACGATTCCATCCAGAAACTGCTGAACGAGAGGAGCCTCTT |
| | | CCGGCAAGCCATCAGCCCCTCCGACAGGGTGAAGCTCTTTCCCCACCG |
| | | GAACTCAAGCAAGTGCAAGTCTAAGCCCCAGATTGCTGCTCTGAAAGA |
| | | GGAGACAGAAGAAGAGGTGCAAGATACAAGGCTTTAGAGAGCAGCATA |
| | | AATGTTGACATGGGACATTTGCTCATGGAATTGGCAGGCCTAATAAAGA |
| | | GCTCAGATGCATCGATCAGAGTGTGTTGGTTTTTTGTGTGTACTGAGGA |
| | | ACCCCTAGTGATGGAGTTGGCCACTCCCTCTCTGCGCGCTCGCTCGCT |
| | | CACTGAGGCCGCCCGGGCAAAGCCCGGGCGTCGGGCGACCTTTGGTC |
| | | GCCCGGCCTCAGTGAGCGAGCGAGCGCGCAGAGAGGGAGTGGCCAA |
| | | CCCCCCCCCCCCCCCCCCTGCAGCCAGCTGGCGTAATAGCGAAGAGG |
| | | CCCGCACCGATCGCCCTTCCCAACAGTTGCGTAGCCTGAATGGCGAAT |
| | | GGCGCGACGCGCCCTGTAGCGGCGCATTAAGCGCGGCGGGTGTGGT |
| | | GGTTACGCGCAGCGTGACCGCTACACTTGCCAGCGCCCTAGCGCCCG |
| | | CTCCTTTCGCTTTCTTCCCTTCCTTTCTCGCCACGTTCGCCGGCTTTCC |
| | | CCGTCAAGCTCTAAATCGGGGGCTCCCTTTAGGGTTCCGATTTAGTGCT |
| | | TTACGGCACCTCGACCCCAAAAAACTTGATTAGGGTGATGGTTCACGTA |
| | | GTGGGCCATCGCCCTGATAGACGGTTTTTCGCCCTTTGACGTTGGAGT |
| | | CCACGTTCTTTAATAGTGGACTCTTGTTCCAAACTGGAACAACACTCAA |
| | | CCCTATCTCGGTCTATTCTTGATTTATAAGGGATTTTGCCGATTTCGG |
| | | CCTATTGGTTAAAAAATGAGCTGATTTAACAAAAATTTAACGCGAATT |
| | | AACAAAATATTAACGTTTACAATTTCCTGATGCGGTATTTTCTCCTTACG |
| | | CATCTGTGCGGTATTTCACACCGCATATGGTGCACTCTCAGTACAATCT |
| | | GCTCTGATGCCGCATAGTTAAGCCAGCCCCGACACCCGCCAACACCCG |
| | | CTGACGCGCCCTGACGGGCTTGTCTGCTCCCGGCATCCGCTTACAGAC |
| | | AAGCTGTGACCGTCTCCGGGAGCTGCATGTGTCAGAGGTTTTCACCGT |
| | | CATCACCGAAACGCGCGAGACGAAAGGGCCTCGTGATACGCCTATTTT |
| | | TATAGGTTAATGTCATGATAATAATGGTTTCTTAGACGTCAGGTGGCACT |
| | | TTTCGGGGAAATGTGCGCGGAACCCCTATTTGTTTATTTTTCTAAATACA |
| | | TTCAAATATGTATCCGCTCATGAGACAATAACCCTGATAAATGCTTCAAT |
| | | AATATTGAAAAAGGAAGAGTATGAGTATTCAACATTTCCGTGTCGCCCT |
| | | TATTCCCTTTTTTGCGGCATTTTGCCTTCCTGTTTTTGCTCACCCAGAAA |
| | | CGCTGGTGAAAGTAAAAGATGCTGAAGATCAGTTGGGTGCACGAGTGG |
| | | GTTACATCGAACTGGATCTCAACAGCGGTAAGATCCTTGAGAGTTTTCG |
| | | CCCCGAAGAACGTTTTCCAATGATGAGCACTTTTAAAGTTCTGCTATGT |
| | | GGCGCGGTATTATCCCGTATTGACGCCGGGCAAGAGCAACTCGGTCG |
| | | CCGCATACACTATTCTCAGAATGACTTGGTTGAGTACTCACCAGTCACA |
| | | GAAAAGCATCTTACGGATGGCATGACAGTAAGAGAATTATGCAGTGCTG |
| | | CCATAACCATGAGTGATAACACTGCGGCCAACTTACTTCTGACAACGAT |
| | | CGGAGGACCGAAGGAGCTAACCGCTTTTTTGCACAACATGGGGGATCA |
| | | TGTAACTCGCCTTGATCGTTGGGAACCGGAGCTGAATGAAGCCATACC |
| | | AAACGACGAGCGTGACACCACGATGCCTGTAGCAATGGCAACAACGTT |
| | | GCGCAAACTATTAACTGGCGAACTACTTACTCTAGCTTCCCGGCAACAA |
| | | TTAATAGACTGGATGGAGGCGGATAAAGTTGCAGGACCACTTCTGCGC |
| | | TCGGCCCTTCCGGCTGGCTGGTTTATTGCTGATAAATCTGGAGCCGGT |
| | | GAGCGTGGGTCTCGCGGTATCATTGCAGCACTGGGGCCAGATGGTAA |
| | | GCCCTCCCGTATCGTAGTTATCTACACGACGGGGAGTCAGGCAACTAT |
| | | GGATGAACGAAATAGACAGATCGCTGAGATAGGTGCCTCACTGATTAA |
| | | GCATTGGTAACTGTCAGACCAAGTTTACTCATATATACTTTAGATTGATT |
| | | TAAAACTTCATTTTTAATTTAAAAGGATCTAGGTGAAGATCCTTTTTGATA |
| | | ATCTCATGACCAAAATCCCTTAACGTGAGTTTTCGTTCCACTGAGCGTC |
| | | AGACCCCGTAGAAAAGATCAAAGGATCTTCTTGAGATCCTTTTTTTCTG |
| | | CGCGTAATCTGCTGCTTGCAAACAAAAAAACCACCGCTACCAGCGGTG |
| | | GTTTGTTTGCCGGATCAAGAGCTACCAACTCTTTTTCCGAAGGTAACTG |
| | | GCTTCAGCAGAGCGCAGATACCAAATACTGTCCTTCTAGTGTAGCCGTA |
| | | GTTAGGCCACCACTTCAAGAACTCTGTAGCACCGCCTACATACCTCGCT |
| | | CTGCTAATCCTGTTACCAGTGGCTGCTGCCAGTGGCGATAAGTCGTGT |
| | | CTTACCGGGTTGGACTCAAGACGATAGTTACCGGATAAGGCGCAGCGG |
| | | TCGGGCTGAACGGGGGGTTCGTGCACACAGCCCAGCTTGGAGCGAAC |
| | | GACCTACACCGAACTGAGATACCTACAGCGTGAGCATTGAGAAAGCGC |
| | | CACGCTTCCCGAAGGGAGAAAGGCGGACAGGTATCCGGTAAGCGGCA |
| | | GGGTCGGAACAGGAGAGCGCACGAGGGAGCTTCCAGGGGGAAACGC |
| | | CTGGTATCTTTATAGTCCTGTCGGGTTTCGCCACCTCTGACTTGAGCGT |
| | | CGATTTTTGTGATGCTCGTCAGGGGGGCGGAGCCTATGGAAAAACGCC |
| | | AGCAACGCGGCCTTTTTACGGTTCCTGGCCTTTTGCTGGCCTTTTGCTC |
| | | ACATGTTCTTTCCTGCGTTATCCCCTGATTCTGTGGATAACCGTATTACC |
| | | GCCTTTGAGTGAGCTGATACCGCTCGCCGCAGCCGAACGACCGAGCG |
| | | CAGCGAGTCAGTGAGCGAGGAAGCGGAAGAGCGCCCAATACGCAAAC |
| | | CGCCTCTCCCCGCGCGTTGGCCGATTCATTAATGCAGCTGGGCTGCAG |
| | | GGGGGGGGGGGGGGGGG |
| 13 | AV.TL65 | MAADGYLPDWLEDTLSEGIRQWWKLKPGPPPPKPAERHKDDSRGLVLPG |
| | capsid | YKYLGPFNGLDKGEPVNEADAAALEHDKAYDRQLDSGDNPYLKYNHADA |
| | protein | EFQERLKEDTSFGGNLGRAVFQAKKRVLEPFGLVEEGAKTAPTGKRIDDH |
| | | FPKRKKARTEEDSKPSTSSDAEAGPSGSQQLQIPAQPASSLGADTMSAGG |
| | | GGPLGDNNQGADGVGNASGDWHCDSTWMGDRWTKSTRTWWLPSYNN |
| | | HQYREIKSGSVDGSNANAYFGYSTPWGYFDFNRFHSHWSPRDWQRLINN |
| | | YWGFRPRSLRVKIFNIQVKEVTVQDSTTTIANNLTSTVQVFTDDDYQLPYW |
| | | GNGTEGCLPAFPPQVFTLPQYGYATLNRDNTENPTERSSFFCLEYFPSKM |
| | | LRTGNNFEFTYNFEEVPFHSSFAPSQNLFKLANPLVDQYLYRFVSTNNTGG |
| | | VQFNKNLAGRYANTYKNWFPGPMGRTQGWNLGSGVNRASVSAFATTNR |
| | | MELEGASYQVPPQPNGMTNNLQGSNTYALENTMIFNSQPANPGTTATYLE |
| | | GNMLITSESETQPVNRVAYNVGGQMATNNQSSTTAPTTGTYNLQEIVPGS |
| | | VWMERDVYLQGPIWAKIPETGAHFHPSPAMGGFGLKHPPPMMLIKNTPVP |
| | | GNITSFSDVPVSSFITQYSTGQVTVEMEWELKKENSKRWNPEIQYTNNYN |
| | | DPQFVDFAPDSTGEYRTTRPIGTRYLTRPL |
| 14 | F5 | GTGGTGAGCGTCTGGGCATGTCTGGGCATGTCTGGGCATGTCTGGGC |
| | enhancer | ATGTCGGGCATTCTGGGCGTCTGGGCATGTCTGGGCATGTCTGGGCAT |
| 15 | 5′ AAV ITR | CCACTCCCTCTCTGCGCGCTCGCTCGCTCACTGAGGCCGCCCGGGCA |
| | (flop) | AAGCCCGGGCGTCGGGCGACCTTTGGTCGCCCGGCCTCAGTGAGCGA |
| | | GCGAGCGCGCAGAGAGGGAGTGGCCAACTCCATCACTAGGGGTTCCT |
| 16 | 3′ AAV ITR | AGGAACCCCTAGTGATGGAGTTGGCCACTCCCTCTCTGCGCGCTCGCT |
| | (flop) | CGCTCACTGAGGCCGGGCGACCAAAGGTCGCCCGACGCCCGGGCTTT |
| | | GCCCGGGCGGCCTCAGTGAGCGAGCGAGCGCGCAGAGAGGGAGTGG |
| | | CC |
| 17 | 5′ AAV ITR | CCACTCCCTCTCTGCGCGCTCGCTCGCTCACTGAGGCCGCCCGGGCA |
| | (flop) | AAGCCCGGGCGTCGGGCGACCTTTGGTCGCCCGGCCTCAGTGAGCGA |
| | through 3 | GCGAGCGCGCAGAGAGGGAGTGGCCAACTCCATCACTAGGGGTTCCT |
| | AAV ITR | CAGATCTGAATTCGTGGTGAGCGTCTGGGCATGTCTGGGCATGTCTGG |
| | (flop) | GCATGTCTGGGCATGTCGGGCATTCTGGGCGTCTGGGCATGTCTGGG |
| | | CATGTCTGGGCATCTCGAGAACGGTGACGTGCACGCGTGGGCGGAGC |
| | | CATCACGCAGGTTGCTATATAAGCAGAGCTCGTTTAGTGAACCGTCAGA |
| | | GTCGAGCCCGAGAGACCATGCAGAGGTCGCCTCTGGAAAAGGCCAGC |
| | | GTTGTCTCCAAACTTTTTTTCAGCTGGACCAGACCAATTTTGAGGAAAG |
| | | GATACAGACAGCGCCTGGAATTGTCAGACATATACCAAATCCCTTCTGT |
| | | TGATTCTGCTGACAATCTATCTGAAAAATTGGAAAGAGAATGGGATAGA |
| | | GAGCTGGCTTCAAAGAAAAATCCTAAACTCATTAATGCCCTTCGGCGAT |
| | | GTTTTTTCTGGAGATTTATGTTCTATGGAATCTTTTTATATTTAGGGGAA |
| | | GTCACCAAAGCAGTACAGCCTCTCTTACTGGGAAGAATCATAGCTTCCT |
| | | ATGACCCGGATAACAAGGAGGAACGCTCTATCGCGATTTATCTAGGCAT |
| | | AGGCTTATGCCTTCTCTTTATTGTGAGGACACTGCTCCTACACCCAGCC |
| | | ATTTTTGGCCTTCATCACATTGGAATGCAGATGAGAATAGCTATGTTTAG |
| | | TTTGATTTATAAGAAGACTTTAAAGCTGTCAAGCCGTGTTCTAGATAAAA |
| | | TAAGTATTGGACAACTTGTTAGTCTCCTTTCCAACAACCTGAACAAATTT |
| | | GATGAAGGACTTGCATTGGCACATTTCGTGTGGATCGCTCCTTTGCAAG |
| | | TGGCACTCCTCATGGGGCTAATCTGGGAGTTGTTACAGGCGTCTGCCT |
| | | TCTGTGGACTTGGTTTCCTGATAGTCCTTGCCCTTTTTCAGGCTGGGCT |
| | | AGGGAGAATGATGATGAAGTACAGAGATCAGAGAGCTGGGAAGATCAG |
| | | TGAAAGACTTGTGATTACCTCAGAAATGATCGAGAACATCCAATCTGTT |
| | | AAGGCATACTGCTGGGAAGAAGCAATGGAAAAAATGATTGAAAACTTAA |
| | | GACAAACAGAACTGAAACTGACTCGGAAGGCAGCCTATGTGAGATACT |
| | | TCAATAGCTCAGCCTTCTTCTTCTCAGGGTTCTTTGTGGTGTTTTTATCT |
| | | GTGCTTCCCTATGCACTAATCAAAGGAATCATCCTCCGGAAAATATTCA |
| | | CCACCATCTCATTCTGCATTGTTCTGCGCATGGCGGTCACTCGGCAATT |
| | | TCCCTGGGCTGTACAAACATGGTATGACTCTCTTGGAGCAATAAACAAA |
| | | ATACAGGATTTCTTACAAAAGCAAGAATATAAGACATTGGAATATAACTT |
| | | AACGACTACAGAAGTAGTGATGGAGAATGTAACAGCCTTCTGGGAGGA |
| | | GGGATTTGGGGAATTATTTGAGAAAGCAAAACAAAACAATAACAATAGA |
| | | AAAACTTCTAATGGTGATGACAGCCTCTTCTTCAGTAATTTCTCACTTCT |
| | | TGGTACTCCTGTCCTGAAAGATATTAATTTCAAGATAGAAAGAGGACAG |
| | | TTGTTGGCGGTTGCTGGATCCACTGGAGCAGGCAAGACTTCACTTCTA |
| | | ATGATGATTATGGGAGAACTGGAGCCTTCAGAGGGTAAAATTAAGCACA |
| | | GTGGAAGAATTTCATTCTGTTCTCAGTTTTCCTGGATTATGCCTGGCAC |
| | | CATTAAAGAAAATATCATCTTTGGTGTTTCCTATGATGAATATAGATACA |
| | | GAAGCGTCATCAAAGCATGCCAACTAGAAGAGGACATCTCCAAGTTTG |
| | | CAGAGAAAGACAATATAGTTCTTGGAGAAGGTGGAATCACACTGAGTG |
| | | GAGGTCAACGAGCAAGAATTTCTTTAGCAAGAGCAGTATACAAAGATGC |
| | | TGATTTGTATTTATTAGACTCTCCTTTTGGATACCTAGATGTTTTAACAGA |
| | | AAAAGAAATATTTGAAAGCTGTGTCTGTAAACTGATGGCTAACAAAACTA |
| | | GGATTGGTCACTTCTAAAATGGAACATTTAAAGAAAGCTGACAAAATA |
| | | TTAATTTTGCATGAAGGTAGCAGCTATTTTTATGGGACATTTTCAGAACT |
| | | CCAAAATCTACAGCCAGACTTTAGCTCAAAACTCATGGGATGTGATTCT |
| | | TTCGACCAATTTAGTGCAGAAAGAAGAAATTCAATCCTAACTGAGACCT |
| | | TACACCGTTTCTCATTAGAAGGAGATGCTCCTGTCTCCTGGACAGAAAC |
| | | AAAAAAACAATCTTTTAAACAGACTGGAGAGTTTGGGGAAAAAAGGAAG |
| | | AATTCTATTCTCAATCCAATCAACTCTACGCTTCAGGCACGAAGGAGGC |
| | | AGTCTGTCCTGAACCTGATGACACACTCAGTTAACCAAGGTCAGAACAT |
| | | TCACCGAAAGACAACAGCATCCACACGAAAAGTGTCACTGGCCCCTCA |
| | | GGCAAACTTGACTGAACTGGATATATATTCAAGAAGGTTATCTCAAGAA |
| | | ACTGGCTTGGAAATAAGTGAAGAAATTAACGAAGAAGACTTAAAGGAGT |
| | | GCCTTTTTGATGATATGGAGAGCATACCAGCAGTGACTACATGGAACAC |
| | | ATACCTTCGATATATTACTGTCCACAAGAGCTTAATTTTTGTGCTAATTT |
| | | GGTGCTTAGTAATTTTTCTGGCAGAGGTGGCTGCTTCTTTGGTTGTGCT |
| | | GTGGCTCCTTGGAAACACTCCTCTTCAAGACAAAGGGAATAGTACTCAT |
| | | AGTAGAAATAACAGCTATGCAGTGATTATCACCAGCACCAGTTCGTATT |
| | | ATGTGTTTTACATTTACGTGGGAGTAGCCGACACTTTGCTTGCTATGGG |
| | | ATTCTTCAGAGGTCTACCACTGGTGCATACTCTAATCACAGTGTCGAAA |
| | | ATTTTACACCACAAAATGTTACATTCTGTTCTTCAAGCACCTATGTCAAC |
| | | CCTCAACACGTTGAAAGCAGGTGGGATTCTTAATAGATTCTCCAAAGAT |
| | | ATAGCAATTTTGGATGACCTTCTGCCTCTTACCATATTTGACTTCATCCA |
| | | GTTGTTATTAATTGTGATTGGAGCTATAGCAGTTGTCGCAGTTTTACAAC |
| | | CCTACATCTTTGTTGCAACAGTGCCAGTGATAGTGGCTTTTATTATGTTG |
| | | AGAGCATATTTCCTCCAAACCTCACAGCAACTCAAACAACTGGAATCTG |
| | | AAGGCAGGAGTCCAATTTTCACTCATCTTGTTACAAGCTTAAAAGGACT |
| | | ATGGACACTTCGTGCCTTCGGACGGCAGCCTTACTTTGAAACTCTGTTC |
| | | CACAAAGCTCTGAATTTACATACTGCCAACTGGTTCTTGTACCTGTCAA |
| | | CACTGCGCTGGTTCCAAATGAGAATAGAAATGATTTTTGTCATCTTCTTC |
| | | ATTGCTGTTACCTTCATTTCCATTTTAACAACAGGAGAAGGAGAAGGAA |
| | | GAGTTGGTATTATCCTGACTTTAGCCATGAATATCATGAGTACATTGCA |
| | | GTGGGCTGTAAACTCCAGCATAGATGTGGATAGCTTGATGCGATCTGT |
| | | GAGCCGAGTCTTTAAGTTCATTGACATGCCAACAGAAGGTAAACCTACC |
| | | AAGTCAACCAAACCATACAAGAATGGCCAACTCTCGAAAGTTATGATTA |
| | | TTGAGAATTCACACGTGAAGAAAGATGACATCTGGCCCTCAGGGGGCC |
| | | AAATGACTGTCAAAGATCTCACAGCAAAATACACAGAAGGTGGAAATGC |
| | | CATATTAGAGAACATTTCCTTCTCAATAAGTCCTGGCCAGAGGGTGGGC |
| | | CTCTTGGGAAGAACTGGATCAGGGAAGAGTACTTTGTTATCAGCTTTTT |
| | | TGAGACTACTGAACACTGAAGGAGAAATCCAGATCGATGGTGTGTCTTG |
| | | GGATTCAATAACTTTGCAACAGTGGAGGAAAGCCTTTGGAGTGATACCA |
| | | CAGAAAGTATTTATTTTTTCTGGAACATTTAGAAAAAACTTGGATCCCTA |
| | | TGAACAGTGGAGTGATCAAGAAATATGGAAAGTTGCAGATGAGGTTGG |
| | | GCTCAGATCTGTGATAGAACAGTTTCCTGGGAAGCTTGACTTTGTCCTT |
| | | GTGGATGGGGGCTGTGTCCTAAGCCATGGCCACAAGCAGTTGATGTGC |
| | | TTGGCTAGATCTGTTCTCAGTAAGGCGAAGATCTTGCTGCTTGATGAAC |
| | | CCAGTGCTCATTTGGATCCAGTAACATACCAAATAATTAGAAGAACTCTA |
| | | AAACAAGCATTTGCTGATTGCACAGTAATTCTCTGTGAACACAGGATAG |
| | | AAGCAATGCTGGAATGCCAACAATTTTTGGTCATAGAAGAGAACAAAGT |
| | | GCGGCAGTACGATTCCATCCAGAAACTGCTGAACGAGAGGAGCCTCTT |
| | | CCGGCAAGCCATCAGCCCCTCCGACAGGGTGAAGCTCTTTCCCCACCG |
| | | GAACTCAAGCAAGTGCAAGTCTAAGCCCCAGATTGCTGCTCTGAAAGA |
| | | GGAGACAGAAGAAGAGGTGCAAGATACAAGGCTTTAGAGAGCAGCATA |
| | | AATGTTGACATGGGACATTTGCTCATGGAATTGGCAGGCCTAATAAAGA |
| | | GCTCAGATGCATCGATCAGAGTGTGTTGGTTTTTTGTGTGTACTGAGGA |
| | | ACCCCTAGTGATGGAGTTGGCCACTCCCTCTCTGCGCGCTCGCTCGCT |
| | | CACTGAGGCCGGGCGACCAAAGGTCGCCCGACGCCCGGGCTTTGCCC |
| | | GGGCGGCCTCAGTGAGCGAGCGAGCGCGCAGAGAGGGAGTGGCC |
| 18 | pAV- | CCACTCCCTCTCTGCGCGCTCGCTCGCTCACTGAGGCCGCCCGGGCA |
| | F5tg83- | AAGCCCGGGCGTCGGGCGACCTTTGGTCGCCCGGCCTCAGTGAGCGA |
| | hCFTR-dR | GCGAGCGCGCAGAGAGGGAGTGGCCAACTCCATCACTAGGGGTTCCT |
| | (flop ITR) | CAGATCTGAATTCGTGGTGAGCGTCTGGGCATGTCTGGGCATGTCTGG |
| | vector | GCATGTCTGGGCATGTCGGGCATTCTGGGCGTCTGGGCATGTCTGGG |
| | | CATGTCTGGGCATCTCGAGAACGGTGACGTGCACGCGTGGGCGGAGC |
| | | CATCACGCAGGTTGCTATATAAGCAGAGCTCGTTTAGTGAACCGTCAGA |
| | | GTCGAGCCCGAGAGACCATGCAGAGGTCGCCTCTGGAAAAGGCCAGC |
| | | GTTGTCTCCAAACTTTTTTTCAGCTGGACCAGACCAATTTTGAGGAAAG |
| | | GATACAGACAGCGCCTGGAATTGTCAGACATATACCAAATCCCTTCTGT |
| | | TGATTCTGCTGACAATCTATCTGAAAAATTGGAAAGAGAATGGGATAGA |
| | | GAGCTGGCTTCAAAGAAAAATCCTAAACTCATTAATGCCCTTCGGCGAT |
| | | GTTTTTTCTGGAGATTTATGTTCTATGGAATCTTTTTATATTTAGGGGAA |
| | | GTCACCAAAGCAGTACAGCCTCTCTTACTGGGAAGAATCATAGCTTCCT |
| | | ATGACCCGGATAACAAGGAGGAACGCTCTATCGCGATTTATCTAGGCAT |
| | | AGGCTTATGCCTTCTCTTTATTGTGAGGACACTGCTCCTACACCCAGCC |
| | | ATTTTTGGCCTTCATCACATTGGAATGCAGATGAGAATAGCTATGTTTAG |
| | | TTTGATTTATAAGAAGACTTTAAAGCTGTCAAGCCGTGTTCTAGATAAAA |
| | | TAAGTATTGGACAACTTGTTAGTCTCCTTTCCAACAACCTGAACAAATTT |
| | | GATGAAGGACTTGCATTGGCACATTTCGTGTGGATCGCTCCTTTGCAAG |
| | | TGGCACTCCTCATGGGGCTAATCTGGGAGTTGTTACAGGCGTCTGCCT |
| | | TCTGTGGACTTGGTTTCCTGATAGTCCTTGCCCTTTTTCAGGCTGGGCT |
| | | AGGGAGAATGATGATGAAGTACAGAGATCAGAGAGCTGGGAAGATCAG |
| | | TGAAAGACTTGTGATTACCTCAGAAATGATCGAGAACATCCAATCTGTT |
| | | AAGGCATACTGCTGGGAAGAAGCAATGGAAAAAATGATTGAAAACTTAA |
| | | GACAAACAGAACTGAAACTGACTCGGAAGGCAGCCTATGTGAGATACT |
| | | TCAATAGCTCAGCCTTCTTCTTCTCAGGGTTCTTTGTGGTGTTTTTATCT |
| | | GTGCTTCCCTATGCACTAATCAAAGGAATCATCCTCCGGAAAATATTCA |
| | | CCACCATCTCATTCTGCATTGTTCTGCGCATGGCGGTCACTCGGCAATT |
| | | TCCCTGGGCTGTACAAACATGGTATGACTCTCTTGGAGCAATAAACAAA |
| | | ATACAGGATTTCTTACAAAAGCAAGAATATAAGACATTGGAATATAACTT |
| | | AACGACTACAGAAGTAGTGATGGAGAATGTAACAGCCTTCTGGGAGGA |
| | | GGGATTTGGGGAATTATTTGAGAAAGCAAAACAAAACAATAACAATAGA |
| | | AAAACTTCTAATGGTGATGACAGCCTCTTCTTCAGTAATTTCTCACTTCT |
| | | TGGTACTCCTGTCCTGAAAGATATTAATTTCAAGATAGAAAGAGGACAG |
| | | TTGTTGGCGGTTGCTGGATCCACTGGAGCAGGCAAGACTTCACTTCTA |
| | | ATGATGATTATGGGAGAACTGGAGCCTTCAGAGGGTAAAATTAAGCACA |
| | | GTGGAAGAATTTCATTCTGTTCTCAGTTTTCCTGGATTATGCCTGGCAC |
| | | CATTAAAGAAAATATCATCTTTGGTGTTTCCTATGATGAATATAGATACA |
| | | GAAGCGTCATCAAAGCATGCCAACTAGAAGAGGACATCTCCAAGTTTG |
| | | CAGAGAAAGACAATATAGTTCTTGGAGAAGGTGGAATCACACTGAGTG |
| | | GAGGTCAACGAGCAAGAATTTCTTTAGCAAGAGCAGTATACAAAGATGC |
| | | TGATTTGTATTTATTAGACTCTCCTTTTGGATACCTAGATGTTTTAACAGA |
| | | AAAAGAAATATTTGAAAGCTGTGTCTGTAAACTGATGGCTAACAAAACTA |
| | | GGATTTTGGTCACTTCTAAAATGGAACATTTAAAGAAAGCTGACAAAATA |
| | | TTAATTTTGCATGAAGGTAGCAGCTATTTTTATGGGACATTTTCAGAACT |
| | | CCAAAATCTACAGCCAGACTTTAGCTCAAAACTCATGGGATGTGATTCT |
| | | TTCGACCAATTTAGTGCAGAAAGAAGAAATTCAATCCTAACTGAGACCT |
| | | TACACCGTTTCTCATTAGAAGGAGATGCTCCTGTCTCCTGGACAGAAAC |
| | | AAAAAAACAATCTTTTAAACAGACTGGAGAGTTTGGGGAAAAAAGGAAG |
| | | AATTCTATTCTCAATCCAATCAACTCTACGCTTCAGGCACGAAGGAGGC |
| | | AGTCTGTCCTGAACCTGATGACACACTCAGTTAACCAAGGTCAGAACAT |
| | | TCACCGAAAGACAACAGCATCCACACGAAAAGTGTCACTGGCCCCTCA |
| | | GGCAAACTTGACTGAACTGGATATATATTCAAGAAGGTTATCTCAAGAA |
| | | ACTGGCTTGGAAATAAGTGAAGAAATTAACGAAGAAGACTTAAAGGAGT |
| | | GCCTTTTTGATGATATGGAGAGCATACCAGCAGTGACTACATGGAACAC |
| | | ATACCTTCGATATATTACTGTCCACAAGAGCTTAATTTTTGTGCTAATTT |
| | | GGTGCTTAGTAATTTTTCTGGCAGAGGTGGCTGCTTCTTTGGTTGTGCT |
| | | GTGGCTCCTTGGAAACACTCCTCTTCAAGACAAAGGGAATAGTACTCAT |
| | | AGTAGAAATAACAGCTATGCAGTGATTATCACCAGCACCAGTTCGTATT |
| | | ATGTGTTTTACATTTACGTGGGAGTAGCCGACACTTTGCTTGCTATGGG |
| | | ATTCTTCAGAGGTCTACCACTGGTGCATACTCTAATCACAGTGTCGAAA |
| | | ATTTTACACCACAAAATGTTACATTCTGTTCTTCAAGCACCTATGTCAAC |
| | | CCTCAACACGTTGAAAGCAGGTGGGATTCTTAATAGATTCTCCAAAGAT |
| | | ATAGCAATTTTGGATGACCTTCTGCCTCTTACCATATTTGACTTCATCCA |
| | | GTTGTTATTAATTGTGATTGGAGCTATAGCAGTTGTCGCAGTTTACAAC |
| | | CCTACATCTTTGTTGCAACAGTGCCAGTGATAGTGGCTTTTATTATGTTG |
| | | AGAGCATATTTCCTCCAAACCTCACAGCAACTCAAACAACTGGAATCTG |
| | | AAGGCAGGAGTCCAATTTTCACTCATCTTGTTACAAGCTTAAAAGGACT |
| | | ATGGACACTTCGTGCCTTCGGACGGCAGCCTTACTTTGAAACTCTGTTC |
| | | CACAAAGCTCTGAATTTACATACTGCCAACTGGTTCTTGTACCTGTCAA |
| | | CACTGCGCTGGTTCCAAATGAGAATAGAAATGATTTTTGTCATCTTCTTC |
| | | ATTGCTGTTACCTTCATTTCCATTTTAACAACAGGAGAAGGAGAAGGAA |
| | | GAGTTGGTATTATCCTGACTTTAGCCATGAATATCATGAGTACATTGCA |
| | | GTGGGCTGTAAACTCCAGCATAGATGTGGATAGCTTGATGCGATCTGT |
| | | GAGCCGAGTCTTTAAGTTCATTGACATGCCAACAGAAGGTAAACCTACC |
| | | AAGTCAACCAAACCATACAAGAATGGCCAACTCTCGAAAGTTATGATTA |
| | | TTGAGAATTCACACGTGAAGAAAGATGACATCTGGCCCTCAGGGGGCC |
| | | AAATGACTGTCAAAGATCTCACAGCAAAATACACAGAAGGTGGAAATGC |
| | | CATATTAGAGAACATTTCCTTCTCAATAAGTCCTGGCCAGAGGGTGGGC |
| | | CTCTTGGGAAGAACTGGATCAGGGAAGAGTACTTTGTTATCAGCTTTTT |
| | | TGAGACTACTGAACACTGAAGGAGAAATCCAGATCGATGGTGTGTCTTG |
| | | GGATTCAATAACTTTGCAACAGTGGAGGAAAGCCTTTGGAGTGATACCA |
| | | CAGAAAGTATTTATTTTTTCTGGAACATTTAGAAAAAACTTGGATCCCTA |
| | | TGAACAGTGGAGTGATCAAGAAATATGGAAAGTTGCAGATGAGGTTGG |
| | | GCTCAGATCTGTGATAGAACAGTTTCCTGGGAAGCTTGACTTTGTCCTT |
| | | GTGGATGGGGGCTGTGTCCTAAGCCATGGCCACAAGCAGTTGATGTGC |
| | | TTGGCTAGATCTGTTCTCAGTAAGGCGAAGATCTTGCTGCTTGATGAAC |
| | | CCAGTGCTCATTTGGATCCAGTAACATACCAAATAATTAGAAGAACTCTA |
| | | AAACAAGCATTTGCTGATTGCACAGTAATTCTCTGTGAACACAGGATAG |
| | | AAGCAATGCTGGAATGCCAACAATTTTTGGTCATAGAAGAGAACAAAGT |
| | | GCGGCAGTACGATTCCATCCAGAAACTGCTGAACGAGAGGAGCCTCTT |
| | | CCGGCAAGCCATCAGCCCCTCCGACAGGGTGAAGCTCTTTCCCCACCG |
| | | GAACTCAAGCAAGTGCAAGTCTAAGCCCCAGATTGCTGCTCTGAAAGA |
| | | GGAGACAGAAGAAGAGGTGCAAGATACAAGGCTTTAGAGAGCAGCATA |
| | | AATGTTGACATGGGACATTTGCTCATGGAATTGGCAGGCCTAATAAAGA |
| | | GCTCAGATGCATCGATCAGAGTGTGTTGGTTTTTTGTGTGTACTGAGGA |
| | | ACCCCTAGTGATGGAGTTGGCCACTCCCTCTCTGCGCGCTCGCTCGCT |
| | | CACTGAGGCCGGGCGACCAAAGGTCGCCCGACGCCCGGGCTTTGCCC |
| | | GGGCGGCCTCAGTGAGCGAGCGAGCGCGCAGAGAGGGAGTGGCCCC |
| | | CCCCCCCCCCCCCCCTGCAGCCTGGCGTAATAGCGAAGAGGCCCGCA |
| | | CCGATCGCCCTTCCCAACAGTTGCGCAGCCTGAATGGCGAATGGACGC |
| | | GCCCTGTAGCGGCGCATTAAGCGCGGCGGGTGTGGTGGTTACGCGCA |
| | | GCGTGACCGCTACACTTGCCAGCGCCCTAGCGCCCGCTCCTTTCGCTT |
| | | TCTTCCCTTCCTTTCTCGCCACGTTCGCCGGCTTTCCCCGTCAAGCTCT |
| | | AAATCGGGGGCTCCCTTTAGGGTTCCGATTTAGTGCTTTACGGCACCTC |
| | | GACCCCAAAAAACTTGATTAGGGTGATGGTTCACGTAGTGGGCCATCG |
| | | CCCTGATAGACGGTTTTTCGCCCTTTGACGTTGGAGTCCACGTTCTTTA |
| | | ATAGTGGACTCTTGTTCCAAACTGGAACAACACTCAACCCTATCTCGGT |
| | | CTATTCTTTTGATTTATAAGGGATTTTGCCGATTTCGGCCTATTGGTTAA |
| | | AAAATGAGCTGATTTAACAAAAATTTAACGCGAATTTTAACAAAATATTAA |
| | | CGCTTACAATTTCCTGATGCGGTATTTTCTCCTTACGCATCTGTGCGGT |
| | | ATTTCACACCGCATATGGTGCACTCTCAGTACAATCTGCTCTGATGCCG |
| | | CATAGTTAAGCCAGCCCCGACACCCGCCAACACCCGCTGACGCGCCCT |
| | | GACGGGCTTGTCTGCTCCCGGCATCCGCTTACAGACAAGCTGTGACCG |
| | | TCTCCGGGAGCTGCATGTGTCAGAGGTTTTCACCGTCATCACCGAAAC |
| | | GCGCGAGACGAAAGGGCCTCGTGATACGCCTATTTTTATAGGTTAATGT |
| | | CATGATAATAATGGTTTCTTAGACGTCAGGTGGCACTTTTCGGGGAAAT |
| | | GTGCGCGGAACCCCTATTTGTTTATTTTTCTAAATACATTCAAATATGTA |
| | | TCCGCTCATGAGACAATAACCCTGATAAATGCTTCAATAATATTGAAAAA |
| | | GGAAGAGTATGAGTATTCAACATTTCCGTGTCGCCCTTATTCCCTTTTTT |
| | | GCGGCATTTTGCCTTCCTGTTTTTGCTCACCCAGAAACGCTGGTGAAAG |
| | | TAAAAGATGCTGAAGATCAGTTGGGTGCACGAGTGGGTTACATCGAAC |
| | | TGGATCTCAACAGCGGTAAGATCCTTGAGAGTTTTCGCCCCGAAGAAC |
| | | GTTTTCCAATGATGAGCACTTTTAAAGTTCTGCTATGTGGCGCGGTATT |
| | | ATCCCGTATTGACGCCGGGCAAGAGCAACTCGGTCGCCGCATACACTA |
| | | TTCTCAGAATGACTTGGTTGAGTACTCACCAGTCACAGAAAAGCATCTT |
| | | ACGGATGGCATGACAGTAAGAGAATTATGCAGTGCTGCCATAACCATG |
| | | AGTGATAACACTGCGGCCAACTTACTTCTGACAACGATCGGAGGACCG |
| | | AAGGAGCTAACCGCTTTTTTGCACAACATGGGGGATCATGTAACTCGCC |
| | | TTGATCGTTGGGAACCGGAGCTGAATGAAGCCATACCAAACGACGAGC |
| | | GTGACACCACGATGCCTGTAGCAATGGCAACAACGTTGCGCAAACTAT |
| | | TAACTGGCGAACTACTTACTCTAGCTTCCCGGCAACAATTAATAGACTG |
| | | GATGGAGGCGGATAAAGTTGCAGGACCACTTCTGCGCTCGGCCCTTCC |
| | | GGCTGGCTGGTTTATTGCTGATAAATCTGGAGCCGGTGAGCGTGGGTC |
| | | TCGCGGTATCATTGCAGCACTGGGGCCAGATGGTAAGCCCTCCCGTAT |
| | | CGTAGTTATCTACACGACGGGGAGTCAGGCAACTATGGATGAACGAAA |
| | | TAGACAGATCGCTGAGATAGGTGCCTCACTGATTAAGCATTGGTAACTG |
| | | TCAGACCAAGTTTACTCATATATACTTTAGATTGATTTAAAACTTCATTTT |
| | | TAATTTAAAAGGATCTAGGTGAAGATCCTTTTTGATAATCTCATGACCAA |
| | | AATCCCTTAACGTGAGTTTTCGTTCCACTGAGCGTCAGACCCCGTAGAA |
| | | AAGATCAAAGGATCTTCTTGAGATCCTTTTTTTCTGCGCGTAATCTGCTG |
| | | CTTGCAAACAAAAAAACCACCGCTACCAGCGGTGGTTTGTTTGCCGGAT |
| | | CAAGAGCTACCAACTCTTTTTCCGAAGGTAACTGGCTTCAGCAGAGCG |
| | | CAGATACCAAATACTGTTCTTCTAGTGTAGCCGTAGTTAGGCCACCACT |
| | | TCAAGAACTCTGTAGCACCGCCTACATACCTCGCTCTGCTAATCCTGTT |
| | | ACCAGTGGCTGCTGCCAGTGGCGATAAGTCGTGTCTTACCGGGTTGGA |
| | | CTCAAGACGATAGTTACCGGATAAGGCGCAGCGGTCGGGCTGAACGG |
| | | GGGGTTCGTGCACACAGCCCAGCTTGGAGCGAACGACCTACACCGAA |
| | | CTGAGATACCTACAGCGTGAGCATTGAGAAAGCGCCACGCTTCCCGAA |
| | | GGGAGAAAGGCGGACAGGTATCCGGTAAGCGGCAGGGTCGGAACAGG |
| | | AGAGCGCACGAGGGAGCTTCCAGGGGGAAACGCCTGGTATCTTTATAG |
| | | TCCTGTCGGGTTTCGCCACCTCTGACTTGAGCGTCGATTTTTGTGATGC |
| | | TCGTCAGGGGGGCGGAGCCTATGGAAAAACGCCAGCAACGCGGCCTT |
| | | TTTACGGTTCCTGGCCTTTTGCTGGCCTTTTGCTCACATGTTCTTTCCTG |
| | | CGTTATCCCCTGATTCTGTGGATAACCGTATTACCGCCTTTGAGTGAGC |
| | | TGATACCGCTCGCCGCAGCCGAACGACCGAGCGCAGCGAGTCAGTGA |
| | | GCGAGGAAGCGGAAGAGCGCCCAATACGCAAACCGCCTCTCCCCGCG |
| | | CGTTGGCCGATTCATTAATGCAGGCTGCAGGGGGGGGGGGGGGGGG |
| | | G |

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method of transducing a recombinant viral vector, the vector being administered to a subject in a dosing regimen comprising at least two doses, the method comprising administering to the subject the recombinant viral vector and an effective amount of an immunosuppressive regimen comprising two or more of a calcineurin inhibitor, a glucocorticoid, an antimetabolite, a mammalian target of rapamycin (mTOR) inhibitor, an alkylating agent, a purine biosynthesis inhibitor, an anti-cluster of differentiation 20 (CD20) antibody, a polyclonal anti-lymphocyte antibody, an immunomodulatory drug, or an immunoglobulin protease.

2. A method of transducing a recombinant viral vector, the vector being administered to a subject in a dosing regimen comprising at least two doses, the method comprising administering to the subject the recombinant viral vector and an effective amount of an immunosuppressive regimen comprising one or more of fingolimod and an immunoglobulin protease.

3. The method of claim 1 or 2, wherein transduction of the recombinant viral vector is improved relative to the transduction level achieved by administration of the recombinant viral vector to the subject in the dosing regimen in the absence of administration of the immunosuppressive regimen.

4. The method of any one of claims 1 to 3, wherein the immunosuppressive regimen improves transduction by inhibiting an immune response in the subject against the viral vector.

5. The method of claim 4, wherein the immune response is an innate immune response, a B-cell mediated immune response, and/or a T-cell mediated immune response.

6. The method of any one of claims 1 to 3, wherein the immunosuppressive regimen improves viral uptake or improves transduction efficiency of the viral vector.

7. A method of improving transgene expression from a recombinant viral vector, the vector being administered to a subject in a dosing regimen comprising at least two doses, the method comprising administering to the subject an effective amount of an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease.

8. A method of improving transgene expression from a recombinant viral vector, the vector being administered to a subject in a dosing regimen comprising at least two doses, the method comprising administering to the subject an effective amount of an immunosuppressive regimen comprising one or more of fingolimod and an immunoglobulin protease.

9. The method of claim 7 or 8, wherein transgene expression from the recombinant viral vector is improved relative to the level of transgene expression achieved by administration of the recombinant viral vector to the subject in the dosing regimen in the absence of administration of the immunosuppressive regimen.

10. The method of claim 9, wherein transgene expression is improved by about 10-fold, about 20-fold, about 30-fold, about 40-fold, about 50-fold, about 60-fold, about 70-fold, about 80-fold, or about 90-fold relative to the level of transgene expression achieved by administration of the recombinant viral vector to the subject in the dosing regimen in the absence of administration of the immunosuppressive regimen.

11. A method of reducing the titer of neutralizing antibodies that bind to a recombinant viral vector, the vector being administered to a subject in a dosing regimen comprising at least two doses, the method comprising administering to the subject an effective amount of an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease.

12. A method of reducing the titer of neutralizing antibodies that bind to a recombinant viral vector, the vector being administered to a subject in a dosing regimen comprising at least two doses, the method comprising administering to the subject an effective amount of an immunosuppressive regimen comprising one or more of fingolimod and an immunoglobulin protease.

13. The method of claim 11 or 12, wherein the titer of neutralizing antibodies is reduced relative to the level of neutralizing antibodies resulting from administration of the recombinant viral vector to the subject in the dosing regimen in the absence of administration of the immunosuppressive regimen.

14. The method of claim 13, wherein the titer of neutralizing antibodies is reduced about 2-fold, about 3-fold, about 4-fold, or about 5-fold relative to the level of neutralizing antibodies resulting from administration of the recombinant viral vector to the subject in the dosing regimen in the absence of administration of the immunosuppressive regimen.

15. The method of any one of claims 1, 3 to 7, 9 to 11, 13, or 14, wherein the immunosuppressive regimen comprises two or more of a calcineurin inhibitor, a glucocorticoid, and an antimetabolite.

16. The method of claim 15, wherein the immunosuppressive regimen comprises a calcineurin inhibitor, a glucocorticoid, and an antimetabolite.

17. The method of any one of claims 1, 3 to 7, 9 to 11, or 13 to 16, wherein the calcineurin inhibitor is cyclosporine, tacrolimus, or a combination thereof.

18. The method of claim 17, wherein the calcineurin inhibitor is cyclosporine.

19. The method of any one of claims 1, 3 to 7, 9 to 11, and 13 to 18, wherein the glucocorticoid is methylprednisolone, prednisolone, hydrocortisone, dexamethasone, cortisone, budesonide, betamethasone, beclomethasone, triamcinolone, or a combination thereof.

20. The method of claim 19, wherein the glucocorticoid is methylprednisolone.

21. The method of any one of claims 1, 3 to 7, 9 to 11, or 13 to 20, wherein the antimetabolite is a purine analogue, a pyrimidine analogue, a nucleoside analogue, a nucleotide analogue, an antifolate, or a combination thereof.

22. The method of claim 21, wherein the purine analogue is azathioprine, mercaptopurine, clofarabine, a thiopurine, fludarabine, pentostatin, cladribine, or a combination thereof.

23. The method of claim 22, wherein the purine analogue is azathioprine.

24. The method of any one of claims 1, 3 to 7, 9 to 11, or 13 to 23, wherein the mTOR inhibitor is rapamycin, everolimus, temsirolimus, ridaforolimus, or a combination thereof.

25. The method of any one of claims 1, 3 to 7, 9 to 11, or 13 to 24, wherein the alkylating agent is cyclophosphamide.

26. The method of any one of claims 1, 3 to 7, 9 to 11, or 13 to 25, wherein the purine biosynthesis inhibitor is mycophenolate mofetil (MMF), mycophenolate sodium, or a combination thereof.

27. The method of any one of claims 1, 3 to 7, 9 to 11, or 13 to 26, wherein the anti-CD20 antibody is rituximab.

28. The method of any one of claims 1, 3 to 7, 9 to 11, or 13 to 27, wherein the polyclonal anti-lymphocyte antibody is anti-thymocyte globulin (ATG).

29. The method of any one of claims 1, 3 to 7, 9 to 11, or 13 to 28, wherein the immunomodulatory drug is fingolimod.

30. The method of any one of claims 1 to 29, wherein the dosing regimen of the recombinant viral vector comprises at least a first dose and a second dose of the recombinant viral vector.

31. The method of claim 30, wherein the second dose of the recombinant viral vector is administered to the subject at least about 4 weeks after the first dose.

32. The method of claim 31, wherein the second dose of the recombinant viral vector is administered to the subject about 4 weeks, about 2 months, about 6 months, or about 12 months after the first dose.

33. The method of claim 32, wherein the second dose of the recombinant viral vector is administered to the subject about 4 weeks after the first dose.

34. The method of any one of claims 30 to 33, wherein the immunosuppressive regimen comprises at least a first dose.

35. The method of claim 34, wherein the first dose of the immunosuppressive regimen is administered to the subject about 1 day, about 2 days, about 3 days, about 4 days, about 5 days, about 6 days, about 7 days, about 2 weeks, about 4 weeks, or about 8 weeks prior to the first dose of the dosing regimen of the recombinant viral vector.

36. The method of claim 35, wherein the first dose of the immunosuppressive regimen is administered to the subject about 2 days prior to the first dose of the dosing regimen of the recombinant viral vector.

37. The method of claim 35, wherein the first dose of the immunosuppressive regimen is administered to the subject on the same day as the first dose of the dosing regimen of the recombinant viral vector.

38. The method of claim 35, wherein the first dose of the immunosuppressive regimen is administered to the subject about 1 day, about 2 days, about 3 days, about 4 days, about 5 days, about 6 days, about 7 days, about 2 weeks, about 3 weeks, about 4 weeks, or about 8 weeks after the first dose of the dosing regimen of the recombinant viral vector.

39. The method of claim 38, wherein the first dose of the immunosuppressive regimen is administered to the subject about 7 days after the first dose of the dosing regimen of the recombinant viral vector.

40. The method of any one of claims 1 to 39, wherein the immunosuppressive regimen is administered to the subject every day, every two days, every three days, every four days, every five days, every six days, every week, every two weeks, every three weeks, every four weeks, every five weeks, every six weeks, every seven weeks, or every eight weeks.

41. The method of claim 40, wherein the immunosuppressive regimen is administered to the subject every day.

42. The method of any one of claims 1 to 41, wherein the recombinant viral vector is a recombinant parvoviral vector, a recombinant retroviral vector, or a recombinant adenoviral vector.

43. The method of claim 42, wherein the recombinant viral vector is a recombinant parvoviral vector.

44. The method of claim 42 or 43, wherein the parvoviral vector is a recombinant adeno-associated virus (rAAV) or a recombinant bocavirus vector.

45. The method of claim 44, wherein the parvoviral vector is an rAAV.

46. The method of any one of claims 43 to 45, wherein the recombinant parvoviral vector comprises a capsid protein and a polynucleotide comprising an enhancer and/or a promoter operably linked to a transgene.

47. The method of claim 46, wherein the capsid protein comprises an AV.TL65 capsid protein, an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AVrh.10 capsid protein, a bocavirus capsid protein, a variant thereof, or a combination thereof.

48. The method of claim 47, wherein the capsid protein is an AV.TL65 capsid protein or a variant thereof.

49. The method of claim 47, wherein the capsid protein is a bocavirus capsid protein.

50. The method of claim 49, wherein the capsid protein is a human bocavirus (HBoV) capsid protein.

51. The method of claim 50, wherein the human bocavirus capsid protein is an HBoV1 capsid protein, an HBoV2 capsid protein, an HBoV3 capsid protein, or an HBoV4 capsid protein.

52. The method of any one of claims 46 to 51, wherein the enhancer comprises an F5 enhancer or a variant thereof.

53. The method of any one of claims 46 to 52, wherein the promoter comprises a tg83 promoter or a variant thereof.

54. The method of any one of claims 46 to 53, wherein the transgene is a therapeutic protein.

55. The method of claim 54, wherein the therapeutic protein is a CFTRΔR minigene or a variant thereof.

56. The method of claim 54, wherein the therapeutic protein is alpha-1 antitrypsin (AAT), surfactant protein (SP)-B, SP-C, or a variant thereof.

57. The method of any one of claims 46 to 56, wherein the recombinant parvoviral vector is an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

58. The method of any one of claims 47, 48, or 57, wherein the AV.TL65 capsid protein comprises the amino acid sequence of SEQ ID NO:13 or the variant comprises a sequence having at least 80% sequence identity to SEQ ID NO:13.

59. The method of claim 52 or 57, wherein the F5 enhancer comprises the polynucleotide sequence of SEQ ID NO:1 or the variant comprises a sequence having at least 80% sequence identity to SEQ ID NO:1.

60. The method of claim 52 or 57, wherein the F5 enhancer comprises the polynucleotide sequence of SEQ ID NO:14 or the variant comprises a sequence having at least 80% sequence identity to SEQ ID NO:14.

61. The method of claim 53 or 57, wherein the tg83 promoter comprises the polynucleotide sequence of SEQ ID NO:2 or the variant comprises a sequence having at least 80% sequence identity to SEQ ID NO:2.

62. The method of claim 55 or 57, wherein the CFTRΔR minigene is a human CFTRΔR minigene.

63. The method of claim 62, wherein the human CFTRΔR minigene is encoded by a polynucleotide comprising the sequence of SEQ ID NO:4 or a variant thereof comprising a sequence having at least 80% sequence identity to SEQ ID NO:4.

64. The method of any one of claims 57 to 63, wherein the polynucleotide comprises, in a 5′-to-3′ direction, the F5 enhancer, the tg83 promoter, and the CFTRΔR minigene.

65. The method of claim 42, wherein the recombinant retroviral vector is a recombinant lentiviral vector.

66. The method of any one of claims 1 to 65, wherein the subject is suffering from a genetic disease, an acquired pulmonary disease, or an infectious disease.

67. The method of claim 66, wherein the genetic disease is cystic fibrosis, AAT deficiency, SP-B deficiency, or SP-C deficiency.

68. The method of claim 66 or 67, wherein the genetic disease is cystic fibrosis.

69. The method of claim 66, wherein the acquired pulmonary disease is chronic obstructive pulmonary disorder (COPD).

70. The method of claim 66, wherein the infectious disease is a viral infection.

71. The method of claim 70, wherein the viral infection is COVID-19.

72. The method of any one of claims 1 to 71, further comprising administering one or more additional therapeutic agents to the subject.

73. The method of claim 72, wherein the one or more additional therapeutic agents includes an augmenter, an antibiotic, a mucus thinner, a CFTR modulator, a mucolytic, normal saline, hypertonic saline, an immunosuppressive agent, or a combination thereof.

74. The method of claim 73, wherein the augmenter comprises an anthracycline, a proteasome inhibitor, a tripeptidyl aldehyde, or a combination thereof.

75. The method of claim 74, wherein the anthracycline comprises doxorubicin, idarubicin, aclarubicin, daunorubicin, epirubicin, calrubicin, mitoxantrone, or a combination thereof.

76. The method of claim 74, wherein the proteasome inhibitor comprises bortezomib (VELCADE®), carfilzomib, ixazomib, or a combination thereof.

77. The method of any one of claims 1 to 76, wherein the recombinant viral vector is administered by inhalation, nebulization, aerosolization, intranasally, intratracheally, intrabronchially, orally, intravenously, subcutaneously, or intramuscularly.

78. The method of claim 77, wherein the recombinant viral vector is administered by inhalation, nebulization, aerosolization, intranasally, intratracheally, and/or intrabronchially.

79. The method of any one of claims 1 to 78, wherein the immunosuppressive regimen is administered intraperitoneally, orally, by inhalation, nebulization, aerosolization, intranasally, intratracheally, intrabronchially, intravenously, subcutaneously, or intramuscularly.

80. The method of any one of claims 1, 3 to 7, 9 to 11, or 13 to 79, wherein the calcineurin inhibitor is administered intraperitoneally.

81. The method of any one of claims 1, 3 to 7, 9 to 11, or 13 to 80, wherein the glucocorticoid is administered intraperitoneally.

82. The method of any one of claims 1, 3 to 7, 9 to 11, or 13 to 81, wherein the antimetabolite is administered orally.

83. A method of administering a recombinant viral vector to a subject, comprising:
(a) administering an effective amount of an immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease to the subject; and
(b) administering a recombinant viral vector to the subject.

84. A method of treating cystic fibrosis in a subject in need thereof, comprising:
(a) administering an effective amount of an immunosuppressive regimen comprising two or more of a calcineurin inhibitor, a glucocorticoid, and an antimetabolite to the subject; and
(b) administering at least a first dose and a second dose of rAAV comprising (i) an AV.TL65 capsid protein; and (ii) a polynucleotide comprising an F5 enhancer and a tg83 promoter operably linked to a CFTRΔR minigene.

85. An immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in improving transduction of a recombinant viral vector that is administered to a subject in a dosing regimen comprising at least two doses.

86. An immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in improving transgene expression from a recombinant viral vector that is administered to a subject in a dosing regimen comprising at least two doses.

87. An immunosuppressive regimen comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in reducing the titer of neutralizing antibodies that bind to a recombinant viral vector that is administered to a subject in a dosing regimen comprising at least two doses.

88. An immunosuppressive regimen comprising two or more of a calcineurin inhibitor, a glucocorticoid, and an antimetabolite for use in treating cystic fibrosis in a subject in need thereof, wherein the immunosuppressive regimen is administered to the subject in combination with at least a first dose and a second dose of rAAV comprising (i) an AV.TL65 capsid protein; and (ii) a polynucleotide comprising an F5 enhancer and a tg83 promoter operably linked to a CFTRΔR minigene.

89. An immunosuppressive regimen comprising one or more of fingolimod and an immunoglobulin protease for use in (i) improving transduction of a recombinant viral vector that is administered to a subject in a dosing regimen comprising at least two doses; (ii) improving transgene expression from a recombinant viral vector that is administered to a subject in a dosing regimen comprising at least two doses; and/or (iii) reducing the titer of neutralizing antibodies that bind to a recombinant viral vector that is administered to a subject in a dosing regimen comprising at least two doses.

90. A kit comprising two or more of a calcineurin inhibitor, a glucocorticoid, an antimetabolite, an mTOR inhibitor, an alkylating agent, a purine biosynthesis inhibitor, an anti-CD20 antibody, a polyclonal anti-lymphocyte antibody, an immunomodulatory drug, or an immunoglobulin protease for use in (i) improving transduction of a recombinant viral vector that is administered to a subject in a dosing regimen comprising at least two doses; (ii) improving transgene expression from a recombinant viral vector that is administered to a subject in a dosing regimen comprising at least two doses; and/or (iii) reducing the titer of neutralizing antibodies that bind to a recombinant viral vector that is administered to a subject in a dosing regimen comprising at least two doses.

91. A kit comprising one or more of fingolimod and an immunoglobulin protease for use in (i) improving transduction of a recombinant viral vector that is administered to a subject in a dosing regimen comprising at least two doses; (ii) improving transgene expression from a recombinant viral vector that is administered to a subject in a dosing regimen comprising at least two doses; and/or (iii) reducing the titer of neutralizing antibodies that bind to a recombinant viral vector that is administered to a subject in a dosing regimen comprising at least two doses.


SEQUENCE LISTING (18 sequences)

SEQ ID NO: 1 - DNA, 107 bp, Artificial Sequence
  A synthetic sequence
gaattcgtggtgagcgtctgggcatgtctgggcatgtctgggcatgtctgggcatgtcgggcattctgggcgtctgggcatgtctgggcatgtctgggcatctcgag

SEQ ID NO: 2 - DNA, 77 bp, Artificial Sequence
  A synthetic sequence
aacggtgacgtgcacgcgtgggcggagccatcacgcaggttgctatataagcagagctcgtttagtgaaccgtcaga

SEQ ID NO: 3 - DNA, 17 bp, Artificial Sequence
  A synthetic sequence
gtcgagcccgagagacc

SEQ ID NO: 4 - DNA, 4287 bp, Artificial Sequence
  A synthetic sequence
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 5 - DNA, 45 bp, Artificial Sequence
  A synthetic sequence
agagcagcataaatgttgacatgggacatttgctcatggaattgg

SEQ ID NO: 6 - DNA, 50 bp, Artificial Sequence
  A synthetic sequence
aataaagagctcagatgcatcgatcagagtgtgttggttttttgtgtgta

SEQ ID NO: 7 - DNA, 4488 bp, Artificial Sequence
  A synthetic sequence
gaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 8 - DNA, 4533 bp, Artificial Sequence
  A synthetic sequence
gaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattgg

SEQ ID NO: 9 - DNA, 145 bp, Artificial Sequence
  A synthetic sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 10 - DNA, 145 bp, Artificial Sequence
  A synthetic sequence
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 11 - DNA, 4890 bp, Artificial Sequence
  A synthetic sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggcaggcctaataaagagctcagatgcatcgatcagagtgtgttggttttttgtgtgtactgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 12 - DNA, 7767 bp, Artificial Sequence
  A synthetic sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggcaggcctaataaagagctcagatgcatcgatcagagtgtgttggttttttgtgtgtactgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcagggggggggggggggggg

SEQ ID NO: 13 - protein, 725 aa, Artificial Sequence
  A synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 14 - DNA, 95 bp, Artificial Sequence
  A synthetic sequence
gtggtgagcgtctgggcatgtctgggcatgtctgggcatgtctgggcatgtcgggcattctgggcgtctgggcatgtctgggcatgtctgggcat

SEQ ID NO: 15 - DNA, 141 bp, Artificial Sequence
  A synthetic sequence
ccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 16 - DNA, 143 bp, Artificial Sequence
  A synthetic sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 17 - DNA, 4884 bp, Artificial Sequence
  A synthetic sequence
ccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggcaggcctaataaagagctcagatgcatcgatcagagtgtgttggttttttgtgtgtactgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 18 - DNA, 6633 bp, Artificial Sequence
  A synthetic sequence
ccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggcaggcctaataaagagctcagatgcatcgatcagagtgtgttggttttttgtgtgtactgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccg